(19) United States
(12) Patent Application Publication
(10) Pub. No.: US 2021/0230261 A1
(43) Pub. Date: Jul. 29, 2021

(54) BUFFERED FORMULATIONS OF BEVACIZUMAB FOR USE OF TREATING DISEASES

(71) Applicant: Outlook Therapeutics, Inc., Cranbury, NJ (US)

(72) Inventors: Chris YONAN, Cranbury, NJ (US); Wiphusanee DENDAMRONGVIT, Cranbury, NJ (US); Martha HEALY-FRIED, Cranbury, NJ (US)

(21) Appl. No.: 17/048,107

(22) PCT Filed: Apr. 17, 2019
(86) PCT No.: PCT/US2019/027790

§ 371 Date: Oct. 16, 2020

Related U.S. Application Data
(60) Provisional application No. 62/658,772, filed on Apr. 17, 2018.
(60) Provisional application No. 62/776,686, filed on Dec. 7, 2018.

(51) Int. Cl. C07K 16/22; A61K 47/26; A61K 9/00; A61K 47/30; A61K 47/08

(52) U.S. Cl. CPC C07K 16/22; A61K 47/26; A61K 2039/505; A61K 47/30; A61K 47/08; A61K 9/0048

(57) ABSTRACT

The invention provides buffered aqueous formulations of bevacizumab. The invention further provides methods of making buffered formulations of bevacizumab. The invention provides methods of treating eye disorders, particularly wet age-related macular degeneration and macular edema by administering the buffered antibody compositions of the disclosure.

### CROSS-REFERENCE TO RELATED APPLICATIONS

This application claims priority to U.S. Provisional Application No. 62/776,686 filed on Dec. 7, 2018 and U.S. Provisional Application No. 62/658,772 filed on Apr. 17, 2018, the contents of each of which are incorporated by reference herein, in their entirety and for all purposes.

### INCORPORATION OF SEQUENCE LISTING

The contents of the text file named “ONBI-013_001WO_SeqListing_ST25.txt,” which was created on Apr. 16, 2019 and is 9 KB in size, are hereby incorporated by reference in their entirety.

### FIELD OF THE INVENTION

The invention relates generally to the field of antibody formulation chemistry. More particularly, the invention relates to buffered formulations of an antibody to vascular endothelial growth factor (VEGF), which formulations enhance the thermal stability and colloidal stability of the antibody, thereby enhancing long-term storage of the antibody. Such stable antibody formulations may be used in methods of treating ocular disorders including those where VEGF is dysregulated.

### BACKGROUND OF THE INVENTION

As part of the Biologics Price Competition and Innovation Act (BPCIA), a biological drug product (produced in or derived from living organisms) may be demonstrated to be “biosimilar” if data show that, among other things, the product is “highly similar” to an already-approved biological product. The biosimilar product should retain at least the biologic function and treatment efficacy of the U.S. Food and Drug Agency-approved biological product. The biosimilar product can be formulated differently, however, from the approved biological product. The formulation can improve stability and shelf storage of the biologic drug product, and can also improve the efficacy in treating a particular disease or condition. The formulation can also improve other aspects of administration, including a reduction in patient discomfort or other untoward effects that a patient may experience upon administration of the approved biological product.

Antibody molecules can be used as biological drugs, and many such antibodies are approved for use in human beings. Antibody molecules can be produced as a biosimilar, and reformulated accordingly. There remains a need in the art for high quality antibody biosimilars.

The bevacizumab antibody marketed under the brand Avastin® (Genentech, Inc., San Francisco, Calif.) is known to aggregate in two forms under storage conditions—a non-covalent, reversible aggregate and a covalent, non-reversible aggregate. It is believed that the latter (covalent aggregate) occurs in the antigen-binding domain and, therefore, reduces the number of binding sites available to bind to vascular endothelial growth factor (VEGF). As a result, the potency of the antibody is diminished. Reduction of such aggregates is desirable generally, and particularly for an antibody such as bevacizumab. The present disclosure addresses these needs in the art.

### SUMMARY OF THE INVENTION

In some embodiments, the disclosure features a method of treating an eye disorder in a subject in need thereof, said method comprising administering to the subject a buffered antibody formulation comprising an antibody comprising a heavy chain comprising the amino acid sequence of SEQ ID NO: 1 and a light chain comprising the amino acid sequence of SEQ ID NO: 2.

In some embodiments of the disclosure, the eye disorder is a disorder of the retina, sclera, vitreous, lens, pupil, iris, cornea, choroid, optic nerve, retinal vasculature, ciliary body, or angle of the eye. In some embodiments of the disclosure, the eye disorder is a disorder of the retina or choroid. In some embodiments of the disclosure, the eye disorder of the retina or choroid is age-related macular degeneration, macular edema, diabetic macular edema (DME), retinopathy, diabetic retinopathy, myopic degeneration, idiopathic choroidal neovascularization, inflammatory choroidal neovascularization, retinal neovascularization, polyploidal choroidal vasculopathy, eye neovascularization, branch retinal vein occlusion (BRVO), central retinal vein occlusion, central serous chorioretinopathy, retinitis, retinitis pigmentosa, stargardt disease, usher syndrome, retinal degeneration, endophthalmitis, familial exudative vitreoretinopathy, idiopathic juxtafoveal telangiectasis, lattice degeneration, macular hole, persistent fetal vasculature, retinal artery occlusion, or retinoblastoma.

In some embodiments of the disclosure, the eye disorder of the retina or choroid is age-related macular degeneration, wet age-related macular degeneration, or neovascular age-related macular degeneration. In some embodiments of the disclosure, the eye disorder of the retina or choroid is wet age-related macular degeneration. In some embodiments of the disclosure, the eye disorder of the retina or choroid is macular edema.

In some embodiments of the disclosure, the buffered antibody formulation is administered to the subject orally, intravenously, intravitreally, intramuscularly, topically, subcutaneously, suprachoroidally, via eye drop, or via direct absorption through mucous membrane tissues. In some embodiments of the disclosure, the buffered antibody formulation is administered to the subject by an intravitreal injection.

In some embodiments of the disclosure, the buffered antibody formulation is administered to the subject 1, 2, 3, 4, 5, 6, 7, 8, 9, 10, 15, 20, 28, 29, 30, or 31 times per month. In some embodiments of the disclosure, the buffered antibody formulation is administered to the subject every 1, 2, 3, 4, 5, 6, 7, 8, 9, 10, 11, or 12 weeks. In some embodiments of the disclosure, the buffered antibody formulation is administered to the subject for a period of time lasting 4, 8, 16, 24, 36, or 52 weeks. In some embodiments of the disclosure, the buffered antibody formulation is administered to the subject for a period of time lasting 1, 2, 3, 4, 5 or 10 years. In some embodiments of the disclosure, the buffered antibody formulation have a half-life of 10 to 50 days.

In some embodiments of the disclosure, the formulation comprises from about 10 mg/ml to about 50 mg/ml of the antibody. In some embodiments of the disclosure, the formulation comprises from about 15 mg/ml to about 35 mg/ml of the antibody. In some embodiments of the disclosure, the formulation comprises from about 23 mg/ml to about 27 mg/ml of the antibody. In some embodiments of the disclosure, the formulation comprises from about 24 mg/ml to about 27 mg/ml of the antibody. In some embodiments of the disclosure, the formulation comprises from about 25 mg/ml to about 26 mg/ml of the antibody. In some embodiments of the disclosure, the formulation comprises about 25.5 mg/ml of the antibody. In some embodiments of the disclosure, the formulation comprises about 25 mg/ml of the antibody.

In some embodiments of the disclosure, the formulation comprises from about 30 mM to about 70 mM of citrate phosphate. In some embodiments of the disclosure, the formulation comprises from about 40 mM to about 60 mM of citrate phosphate. In some embodiments of the disclosure, the formulation comprises from about 48 mM to about 52 mM of citrate phosphate. In some embodiments of the disclosure, the formulation comprises from about 49 mM to about 51 mM of citrate phosphate. In some embodiments of the disclosure, the formulation comprises from about 50 mM to about 51 mM of citrate phosphate.

In some embodiments of the disclosure, the formulation comprises from about 30 mM to about 70 mM of sodium phosphate. In some embodiments of the disclosure, the formulation comprises from about 40 mM to about 60 mM of sodium phosphate. In some embodiments of the disclosure, the formulation comprises from about 48 mM to about 52 mM of sodium phosphate. In some embodiments of the disclosure, the formulation comprises from about 49 mM to about 51 mM of sodium phosphate. In some embodiments of the disclosure, the formulation comprises from about 50 mM to about 51 mM of sodium phosphate.

In some embodiments of the disclosure, the formulation comprises sodium phosphate monobasic, sodium phosphate dibasic, or both sodium phosphate monobasic and sodium phosphate dibasic. In some embodiments of the disclosure, the buffer comprises about 50 mM of sodium phosphate. In some embodiments of the disclosure, the buffer comprises about 51 mM of sodium phosphate.

In some embodiments of the disclosure, the formulation comprises from about 120 mM to about 180 mM of trehalose. In some embodiments of the disclosure, the formulation comprises from about 140 mM to about 180 mM of trehalose. In some embodiments of the disclosure, the formulation comprises from about 150 mM to about 170 mM of trehalose. In some embodiments of the disclosure, the formulation comprises from about 157 mM to about 161 mM of trehalose. In some embodiments of the disclosure, the formulation comprises from about 158 mM to about 160 mM of trehalose. In some embodiments of the disclosure, the formulation comprises about 159 mM of trehalose. In some embodiments of the disclosure, the formulation comprises about 160 mM of trehalose.

In some embodiments of the disclosure, the formulation comprises from about 0.02% (v/v) to about 0.06% (v/v) of polysorbate 20. In some embodiments of the disclosure, the formulation comprises from about 0.03% (v/v) to about 0.05% (v/v) of polysorbate 20. In some embodiments of the disclosure, the formulation comprises about 0.04% (v/v) of polysorbate 20.

In some embodiments of the disclosure, the formulation has a pH of about 5.6. In some embodiments of the disclosure, the formulation has a pH of about 5.8. In some embodiments of the disclosure, the formulation has a pH of about 6. In some embodiments of the disclosure, the formulation has a pH of about 6.1.

In some embodiments of the disclosure, the buffer comprises from about 11 mM to about 19 mM of sodium acetate. In some embodiments of the disclosure, the buffer comprises from about 13 mM to about 17 mM of sodium acetate. In some embodiments of the disclosure, the buffer comprises from about 13 mM to about 16 mM of sodium acetate. In some embodiments of the disclosure, the buffer comprises about 15 mM of sodium acetate.

In some embodiments of the disclosure, the formulation comprises from about 165 mM to about 185 mM of sucrose. In some embodiments of the disclosure, the formulation comprises from about 170 mM to about 180 mM of sucrose. In some embodiments of the disclosure, the formulation comprises from about 174 mM to about 176 mM of sucrose. In some embodiments of the disclosure, the formulation comprises about 175 mM of sucrose.

In some embodiments, the disclosure features a kit comprising: a) a buffered antibody formulation comprising an antibody comprising a heavy chain comprising the amino acid sequence of SEQ ID NO: 1 and a light chain comprising the amino acid sequence of SEQ ID NO: 2; and b) instructions for administering the antibody formulation in a method for treating an eye disorder. In some embodiments of the disclosure, the eye disorder is a disorder of the retina, sclera, vitreous, lens, pupil, iris, cornea, choroid, optic nerve, retinal vasculature, ciliary body, or angle of the eye. In some embodiments of the disclosure, the eye disorder is a disorder of the retina or choroid. In some embodiments of the disclosure, the eye disorder of the retina or choroid is age-related macular degeneration, macular edema, diabetic macular edema (DME), retinopathy, diabetic retinopathy, myopic degeneration, idiopathic choroidal neovascularization, inflammatory choroidal neovascularization, retinal neovascularization, polyploidal choroidal vasculopathy, eye neovascularization, branch retinal vein occlusion (BRVO), central retinal vein occlusion, central serous chorioretinopathy, retinitis, retinitis pigmentosa, stargardt disease, usher syndrome, retinal degeneration, endophthalmitis, familial exudative vitreoretinopathy, idiopathic juxtafoveal telangiectasis, lattice degeneration, macular hole, persistent fetal vasculature, retinal artery occlusion, or retinoblastoma. In some embodiments of the disclosure, the eye disorder of the retina or choroid is age-related macular degeneration, wet age-related macular degeneration, or neovascular age-related macular degeneration. In some embodiments of the disclosure, the eye disorder of the retina or choroid is wet age-related macular degeneration.

In some embodiments of the disclosure, the instructions include instructions for administering the stable antibody as described in any one of claims 1-56. In some embodiments of the disclosure, the kit further comprises a device for injecting the buffered antibody formulation selected from the group comprising: a syringe, needle, and catheter.

### BRIEF DESCRIPTION OF THE DRAWINGS

FIG. 1 shows a DSC plot showing the effect of various stabilizers on bevacizumab thermal stability in a 50 mM sodium phosphate buffer. Conditions 1, 2, 9, and 10 from Table 1 are shown in the plot.

FIG. 2A shows the percent of bevacizumab aggregates in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when stored at 5° C. over a duration of 18 months.

FIG. 2A (i): shows the chromatographic overlay (as measured by size exclusion chromatography (SEC) using neat injection conditions and quantifies total aggregates) for Condition 1 (Bevacizumab (Avastin®) Match), Condition 2 (Bevacizumab Citrate Phosphate, pH 5.8) and Condition 3 (Bevacizumab Citrate Phosphate, pH 6.0) when product is stored at 5° C. over a duration of 18 months.

FIG. 2A (ii) shows the chromatographic overlay (as measured by size exclusion chromatography (SEC) using neat injection conditions and quantifies total aggregates) for Condition 1 (Bevacizumab (Avastin®) Match), Condition 4 (Bevacizumab Acetate, pH 5.6), and Condition 5 (Bevacizumab Acetate, pH 5.8) when product is stored at 5° C. over a duration of 18 months.

FIG. 2B shows the percent of bevacizumab covalent dimer in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when stored at 5° C. over a duration of 18 months.

FIG. 2B (i) shows the chromatographic overlay (as measured by size exclusion chromatography (SEC) using dilute injection conditions and quantifies bevacizumab covalent dimer) for Condition 1 (Bevacizumab (Avastin®) Match), Condition 2 (Bevacizumab Citrate Phosphate, pH 5.8) and Condition 3 (Bevacizumab Citrate Phosphate, pH 6.0) when product is stored at 5° C. over 18 months.

FIG. 2B (ii) shows the chromatographic overlay (as measured by Size Exclusion Chromatography (SEC) using dilute injection conditions and quantifies the bevacizumab covalent dimer) for Condition 1 (Bevacizumab (Avastin®) Match), Condition 4 (Bevacizumab Acetate, pH 5.6), and Condition 5 (Bevacizumab Acetate, pH 5.8) when product is stored at 5° C. over 18 months.

FIG. 2B (iii) shows the changes in percent of bevacizumab acidic species as measured by cation exchange chromatography (CEX) in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when stored at 5° C. over 18 months.

FIG. 2B (iv) shows the chromatographic overlay (as measured by cation exchange chromatography (CEX) and quantifies % acidic, % basic and % main species) for Condition 1 (Bevacizumab (Avastin®) Match), Condition 2 (Bevacizumab Citrate Phosphate, pH 5.8) and Condition 3 (Bevacizumab Citrate Phosphate, pH 6.0) when product is stored at 5° C. over 18 months.

FIG. 2B (v) shows the chromatographic overlay (as measured by cation exchange chromatography (CEX) and quantifies % acidic, % basic and % main species) for Condition 1 (Bevacizumab (Avastin®) Match), Condition 4 (Bevacizumab Acetate, pH 5.6), and Condition 5 (Bevacizumab Acetate, pH 5.8) when product is stored at 5° C. over 18 months.

FIG. 2C shows the percent of bevacizumab aggregates in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when stored at 30° C.

FIG. 2D shows the percent of bevacizumab covalent dimer in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when stored at 30° C.

FIG. 2E shows the percent of bevacizumab aggregates in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when stored at 37° C.

FIG. 2F shows the percent of bevacizumab covalent dimer in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when stored at 37° C.

FIG. 2G shows the percent of bevacizumab aggregates in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when subject to shaking stress.

FIG. 2H shows the percent of bevacizumab covalent dimer in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when subject to shaking stress.

FIG. 2I shows the percent of bevacizumab aggregates in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when subject to freeze/thaw stress.

FIG. 2J shows the percent of bevacizumab covalent dimer in Condition 1 (Bevacizumab (Avastin®) Match), Condition 2 (Bevacizumab Citrate Phosphate, pH 5.8), Condition 3 (Bevacizumab Citrate Phosphate, pH 6.0), Condition 4 (Bevacizumab Acetate, pH 5.6), and Condition 5 (Bevacizumab Acetate, pH 5.8), when subject to freeze/thaw stress.

FIG. 3 shows the hydrodynamic size of bevacizumab with changing concentration.

FIG. 4 shows an accelerated stability T=0 Intrinsic Fluorescence Emission Scan Tryptophan plot.

FIG. 5 shows a flow diagram of the process of manufacturing the stable antibody composition ONS-5010. The Ultrafiltration and Diafiltration (UF/DF) process is the penultimate step in manufacturing the stable antibody composition.

FIG. 6A shows protein concentration and % HMWS of intermediate material in the UF/DF process in the first experiment. The bar on the left of each pair of bars indicates protein concentration. The bar on the right of each pair of bars indicates % HMWS.

FIG. 6B shows protein concentration and % HMWS of intermediate material in the UF/DF process in the second run. The bar on the left of each pair of bars indicates protein concentration. The bar on the right of each pair of bars indicates % HMWS.

FIG. 6C shows protein concentration and % HMWS of intermediate material in the UF/DF process in the third run. The bar on the left of each pair of bars indicates protein concentration. The bar on the right of each pair of bars indicates % HMWS.

FIG. 7 shows changes in % HMWS associated with titration of the stable antibody composition to a final pH of 6.1, and following a final 0.2 μm filtration, as measured by size exclusion HPLC (SE-HPLC).

FIG. 8 shows changes in % HMWS associated with diafiltration in 6% (w/v) α,α′ trehalose in water followed by addition of a 0.5 M sodium phosphate solution, and a 10% polysorbate 20 solution to a composition of 5.8 g/L sodium phosphate monobasic monohydrate, 1.2 g/L sodium phosphate dibasic, anhydrous, 60.0 g/L α,α′ trehalose, 0.04% polysorbate 20.

FIG. 9 shows the effect of pH of the bulk drug substance (BDS) on the % HMWS. The bar on the left of each set of bars indicates HEPES. The bar in the middle of each set of bars indicates phosphate. The bar on the right of each set of bars indicates hydroxide.

FIG. 10 shows the effect of varying the concentration of ONS-5010 retentate on % HMWS. The fourth bar (right-most) of the middle set of bars (28 g/L) represents the aliquot in which the phosphate adjustment was performed with solid mono- and di-basic sodium phosphate, rather than a stock solution.

FIG. 11 shows a flow diagram of the UF/DF process for manufacturing ONS-5010.

FIG. 12 shows the initial permeate flux versus retentate pressure curves for five feed flow rates. The lines on the graph from top to bottom represent 500 LMH, 400 LMH, 300 LMH, 200 LMH, and 100 LMH.

FIG. 13 shows the concentrated flux versus retentate pressure curves for five feed flow rates in the starting buffer (25 mM sodium acetate, 237 mM sodium chloride, pH 5.0). The lines on the graph from top to bottom represent 500 LMH, 400 LMH, 300 LMH, 200 LMH, and 100 LMH.

FIG. 14 shows the concentrated flux versus retentate pressure curves for five feed flow rates in the final buffer. The lines on the graph from top to bottom represent 500 LMH, 400 LMH, 300 LMH, 200 LMH, and 100 LMH.

FIG. 15 shows the impact of concentration of the stable antibody composition on the diafiltration optimization. The lines on the graph from top to bottom represent final buffer (51 mM sodium phosphate, 0.04% polysorbate, pH 6.1) and starting buffer (25 mM sodium acetate, 237 mM sodium chloride, pH 5.0).

FIG. 16 demonstrates the concentration-time profile of ONS-5010, U.S.-licensed Avastin, and E.U.-licensed Avastin as the mean. The vertical line at time zero denotes dosing.

### DETAILED DESCRIPTION OF THE INVENTION

The invention features buffered formulations for storage of bevacizumab. The bevacizumab may comprise a heavy chain comprising the amino acid sequence of SEQ ID NO: 1 and a light chain comprising the amino acid sequence of SEQ ID NO: 2, or heavy chain variable region comprising the amino acid sequence of SEQ ID NO: 3 and a light chain variable region comprising the amino acid sequence of SEQ TD NO: 4. In the buffered formulation, bevacizumab may be present in a concentration of from about 10 mg to about 50 mg, or more preferably from about 15 mg/ml to about 35 mg/ml, or more preferably from about 24 mg/ml to about 27 mg/ml, or more preferably about 25 mg/ml or about 25.5 mg/ml. The formulation is aqueous, and the buffer may comprise citrate phosphate or sodium acetate, and the formulation may also comprise a stabilizer that comprises a sugar such as trehalose or sucrose, as well as a mild surfactant such as polysorbate 20. The formulation preferably has an acidic pH of from about 5.6 to about 6.1, and in some aspects, the pH is about 5.6, or about 5.8, or about 6.

In some aspects, the formulation comprises a buffer comprising from about 10 mM to about 100 mM of citrate phosphate, from about 100 mM to about 200 mM of trehalose, and from about 0.01% (v/v) to about 0.1% (v/v) of polysorbate 20, and has a pH of from about 5.7 to about 6.1. The citrate phosphate may be at a concentration range of from about 30 mM to about 70 mM, from about 40 mM to about 60 mM, from about 48 mM to about 52 mM, from about 49 mM to about 51 mM, or from about 50 mM to about 51 mM, or may be at a concentration of about 50 mM or about 51 mM. The trehalose may be at a concentration range of from about 120 mM to about 180 mM, from about 150 mM to about 170 mM, from about 157 mM to about 161 mM, from about 140 mM to about 180 mM, or from about 158 mM to about 160 mM, or at a concentration of about 159 mM or about 160 mM. The polysorbate may be at a concentration range of from about 0.02% (v/v) to about 0.06% (v/v), or from about 0.03% (v/v) to about 0.05% (v/v), or may be at a concentration of about 0.04% (v/v). The formulation pH may be about 5.8 or may be about 6.

In some aspects, the formulation comprises a buffer comprising from about 5 mM to about 25 mM of sodium acetate trihydrate, from about 150 mM to about 201 mM of sucrose, and from about 0.03% (v/v) to about 0.05% (v/v) of polysorbate 20, and has a pH of from about 5.5 to about 5.9. The sodium acetate trihydrate may be at a concentration range of from about 11 mM to about 19 mM, from about 13 mM to about 17 mM, or from about 13 mM to about 16 mM, or may be at a concentration of about 15 mM. The sucrose may be at a concentration range of from about 165 mM to about 185 mM, from about 170 mM to about 180 mM, or from about 174 mM to about 176 mM, or may be at a concentration of about 175 mM.

The present disclosure provides a buffered antibody formulation, comprising an antibody comprising a heavy chain comprising the amino acid sequence of SEQ ID NO: 1 and a light chain comprising the amino acid sequence of SEQ ID NO: 2, a buffer comprising from about 10 mM to about 100 mM of citrate phosphate, from about 100 mM to about 200 mM of trehalose, and from about 0.01% (v/v) to about 0.1% (v/v) of polysorbate 20, wherein the antibody formulation has a pH of from about 5.7 to about 6.1. Preferably, the antibody formulation is stable for at least 18 months when stored under refrigerated conditions at 5° C.

The present disclosure also provides a buffered antibody formulation, comprising from about 15 mg/ml to about 35 mg/ml of an antibody comprising a heavy chain comprising the amino acid sequence of SEQ ID NO: 1 and a light chain comprising the amino acid sequence of SEQ ID NO: 2, a buffer comprising from about 40 mM to about 60 mM of citrate phosphate, from about 140 mM to about 180 mM of trehalose, and from about 0.02% (v/v) to about 0.06% (v/v) of polysorbate 20, wherein the antibody formulation has a pH of from about 5.7 to about 6.1. Preferably, the antibody formulation is stable for at least 18 months when stored under refrigerated conditions at 5° C.

The present disclosure provides a buffered antibody formulation, comprising from about 24 mg/ml to about 27 mg/ml of an antibody comprising a heavy chain comprising the amino acid sequence of SEQ ID NO: 1 and a light chain comprising the amino acid sequence of SEQ ID NO: 2, a buffer comprising from about 48 mM to about 52 mM of citrate phosphate, from about 157 mM to about 161 mM of trehalose, and from about 0.03% (v/v) to about 0.05% (v/v) of polysorbate 20, wherein the antibody formulation has a pH of from about 5.8 to about 6.0. Preferably, the antibody formulation is stable for at least 18 months when stored under refrigerated conditions at 5° C.

The present disclosure provides a buffered antibody formulation, comprising an antibody comprising a heavy chain comprising the amino acid sequence of SEQ ID NO: 1 and a light chain comprising the amino acid sequence of SEQ ID NO: 2, a buffer comprising about 50 mM of citrate phosphate, about 159 mM of trehalose, and about 0.04% (v/v) of polysorbate 20, wherein the antibody formulation has a pH of about 5.8 or about 6. Preferably, the antibody formulation is stable for at least 18 months when stored under refrigerated conditions at 5° C.

The present disclosure also provides a buffered antibody formulation, comprising from about 20 mg/ml to about 30 mg/ml of an antibody comprising a heavy chain comprising the amino acid sequence of SEQ ID NO: 1 and a light chain comprising the amino acid sequence of SEQ ID NO: 2, a buffer comprising from about 5 mM to about 25 mM of sodium acetate, from about 150 mM to about 201 mM of sucrose, and from about 0.03% (v/v) to about 0.05% (v/v) of polysorbate 20, wherein the antibody formulation has a pH of from about 5.6 to about 5.8. Preferably, the antibody formulation is stable for at least 18 months when stored under refrigerated conditions at 5° C.

The present disclosure also provides a buffered antibody formulation, comprising from about 24 mg/ml to about 26 mg/ml of an antibody comprising a heavy chain comprising the amino acid sequence of SEQ ID NO: 1 and a light chain comprising the amino acid sequence of SEQ ID NO: 2, a buffer comprising from about 13 mM to about 17 mM of sodium acetate, from about 170 mM to about 180 mM of sucrose, and from about 0.03% (v/v) to about 0.05% (v/v) of polysorbate 20, wherein the antibody formulation has a pH of from about 5.6 to about 5.8. Preferably, the antibody formulation is stable for at least 18 months when stored under refrigerated conditions at 5° C.

The present disclosure also provides a buffered antibody formulation, comprising an antibody comprising a heavy chain comprising the amino acid sequence of SEQ ID NO: 1 and a light chain comprising the amino acid sequence of SEQ ID NO: 2, a buffer comprising about 15 mM of sodium acetate, about 175 mM of sucrose, and about 0.04% (v/v) of polysorbate 20, wherein the antibody formulation has a pH of about 5.6 or about 5.8. Preferably, the antibody formulation is stable for at least 18 months when stored under refrigerated conditions at 5° C.

The present disclosure also provides a buffered antibody formulation, comprising an antibody comprising a heavy chain comprising the amino acid sequence of SEQ ID NO: 1 and a light chain comprising the amino acid sequence of SEQ ID NO: 2, a buffer comprising about 15 mM of sodium acetate, about 175 mM of sucrose, and about 0.04% (v/v) of polysorbate 20, wherein the antibody formulation has a pH of about 5.6 or about 5.8. Preferably, the antibody formulation is stable for at least 18 months when stored under refrigerated conditions at 5° C.

The present disclosure also provides a kit comprising any buffered antibody formulation, comprising an antibody comprising a heavy chain comprising the amino acid sequence of SEQ ID NO: 1 and a light chain comprising the amino acid sequence of SEQ ID NO: 2 disclosed herein. The kit can further comprise a device for injecting the antibody formulation into a subject. The device can comprise a syringe, a needle, a catheter, or any combination thereof. The kit can further comprise instructions for treating one or more of the cancers disclosed herein.

The present disclosure also provides methods for treating cancer in a subject in need thereof, the method comprising administering to the subject any buffered antibody formulation, comprising an antibody comprising a heavy chain comprising the amino acid sequence of SEQ ID NO: 1 and a light chain comprising the amino acid sequence of SEQ ID NO: 2 disclosed herein in an amount effective to treat said cancer.

The present disclosure also provides any buffered antibody formulation, comprising an antibody comprising a heavy chain comprising the amino acid sequence of SEQ ID NO: 1 and a light chain comprising the amino acid sequence of SEQ ID NO: 2 disclosed herein for use in the manufacture of a medicament for the treatment of cancer.

Any of the antibody formulations can used in a method for treating one or more of platinum-resistant recurrent epithelial ovarian, fallopian tube, or primary peritoneal cancer. In general, the methods comprise administering the formulation, including the bevacizumab antibody, to a subject in need thereof, in an amount effective to treat one or more of platinum-resistant recurrent epithelial ovarian, fallopian tube, or primary peritoneal cancer. The subject is preferably a human being, and the formulation is preferably administered via intravenous infusion or injection. Any of the antibody formulations may similarly be used in the manufacture of a medicament for the treatment of cancer such as one or more of platinum-resistant recurrent epithelial ovarian, fallopian tube, or primary peritoneal cancer, persistent, recurrent or metastatic cervical cancer, metastatic colorectal cancer, metastatic HER2 (human epidermal growth factor receptor 2) negative breast cancer, metastatic renal cell carcinoma, glioblastoma, or non-small cell lung cancer.

Unless defined otherwise, all technical and scientific terms used herein have the same meaning as commonly understood by one of ordinary skill in the art to which this invention belongs. All patents and publications cited herein are incorporated by reference in their entirety and for all purposes.

In some embodiments, the disclosure features a method of producing a stable antibody composition. In some embodiments, the method comprises ultrafiltering a starting composition, e.g., a starting composition having a pH of about 4.7 to about 5.3. In some embodiments, the starting composition comprises, consists essentially of, or consists of an antibody and a starting buffer composition. In some embodiments, the starting composition comprises, consists essentially of, or consists of between or between about 4 mg/ml and 6 mg/ml of an antibody, such as an antibody comprising a heavy chain comprising the amino acid sequence of SEQ ID NO:1 and a light chain comprising the amino acid sequence of SEQ ID NO: 2. In some embodiments, ultrafiltration of the starting composition produces a concentrated composition. In some embodiments, the concentrated composition comprises between or between about 30 g/L and 40 g/L, inclusive of the endpoints, of the antibody. In some embodiments, the pH of the concentrated composition is between or between about 4.0 and 6.0, such as between or between about 4.5 and 5.5. In some embodiments, the pH of the concentrated composition is or is about 5.0.

In some embodiments, the method comprises exchanging (e.g., diafiltering) the starting buffer composition with an exchange solution comprising 6% trehalose (w/v) in water to produce a trehalose composition. In some embodiments, the trehalose composition comprises between or between about 20 g/L and 50 g/L, between or between about 30 and 40 g/L, or about 35 g/L of the antibody, inclusive of the endpoints. In some embodiments, the trehalose composition comprises between about 30 g/L and about 40 g/L of the antibody, inclusive of the endpoints. In some embodiments, the pH of the trehalose composition is between or between about 4.0 and 6.0, such as between or between about 4.5 and 5.5. In some embodiments, the pH of the trehalose composition is or is about 5.0.

In some embodiments, the method comprises contacting the trehalose composition with a phosphate composition to produce a pH adjusted composition. In some embodiments, the phosphate composition comprises between or between about 400 and 600 mM sodium phosphate, such as between or between about 450 and 550 mM sodium phosphate. In some embodiments, the phosphate composition comprises about 500 mM sodium phosphate. In some embodiments, a final concentration of sodium phosphate in the pH adjusted composition is between or between about 40 and 60 mM, such as between or between about 45 and 55 mM. In some embodiments, the final concentration of sodium phosphate in the pH adjusted composition is about 50 mM. In some embodiments, the pH of the pH adjusted composition is between or between about 5.0 and 7.0, such as between or between about 5.9 and 6.3, inclusive of the endpoints. In some embodiments, the pH of the pH adjusted composition is about 6.1.

In some embodiments, the method comprises formulating the pH adjusted composition for delivery to a subject, thereby producing a stable antibody composition. In some embodiments, the stable antibody composition comprises: 15% high molecular weight species (HMWS), such as ≤10%, ≤7.5%, ≤6%, ≤5%, ≤4%, ≤3%, ≤2.5%, ≤2%, ≤1.5%, or ≤1% HMWS. In some embodiments, the stable antibody composition comprises ≤6% HMWS.

In some embodiments, the stable antibody composition may be referred to as ONS-5010.

In some embodiments, the starting composition has a pH of between or between about 4.0 and 6.0, such as between or between about 4.5 and 5.5, or between about 4.7 and 5.3. In some embodiments, the starting composition has a pH of about 5.0.

In some embodiments, the starting buffer composition comprises between or between about 10 and 40 mM sodium acetate, such as between or between about 15 and 35 mM sodium acetate, or between or between about 20 and 30 mM sodium acetate. In some embodiments, the starting buffer composition comprises about 25 mM sodium acetate. In some embodiments, the starting buffer comprises between or between about 200 and 300 mM NaCl, such as between or between about 225 and 240 mM NaCl. In some embodiments, the starting buffer composition comprises about 237 mM NaCl. In some embodiments, the pH of the starting buffer composition is between or between about 4.0 and 6.0, such as between or between about 4.5 and 5.5. In some embodiments, the pH of the starting buffer composition is about 5.0.

In some embodiments, the ultrafiltering comprises the use of a 30 kDA membrane. In some embodiments, said membrane is a polyethersulfone membrane. In some embodiments, the membrane bears a load of ≤1000 g/m², ≤750 g/m², ≤500 g/m², or ≤250 g/m². In some embodiments, the membrane bears a load of ≤about 500 g/m² to ≤about 100 g/m². In some embodiments, the membrane bears a load of ≤about 300 g/m². In some embodiments, the ultrafiltering has a feed flow rate of ≤about 450 LMH. In some embodiments, the ultrafiltering has a feed flow rate of about 375 LMH. In some embodiments, the ultrafiltering has a retentate pressure of ≤about 25 psi. In some embodiments, the ultrafiltering has a retentate pressure of 5 psi or about 5 psi. In some embodiments, the ultrafiltering has a transmembrane pressure (TMP) of ≤about 20 psi. In some embodiments, the ultrafiltering has a transmembrane pressure (TMP) of about 15 psi.

In some embodiments, the concentrated composition comprises between or between about 20 and 50 mg/ml of the antibody, such as between or between about 25 and 45 mg/ml, or between or between about 30 and 40 mg/ml. In some embodiments, the concentrated composition comprises 35 mg/ml of the antibody. In some embodiments, the pH of the concentrated composition is between or between about 4.0 and 6.0, such as between or between about 4.5 and 5.5. In some embodiments, the pH of the concentrated composition is or is about 5.0.

The disclosure features a method of producing a stable antibody composition comprising exchanging the starting buffer composition with an exchange solution comprising, consisting essentially of, or consisting of 6% trehalose (w/v) in water to produce a trehalose composition. In some embodiments, the trehalose composition comprises between about 30 g/L and about 40 g/L, inclusive of the endpoints, of the antibody. In some embodiments, the pH of the trehalose composition is between or between about 4.0 and 6.0, such as between or between about 4.5 and 5.5. In some embodiments, the pH of the trehalose composition is about 5.0.

In some embodiments, the exchange solution comprises 6% (w/v) α, α′-trehalose in water. In some embodiments, the pH of the exchange composition is between or between about 4.0 and 6.0, such as between or between about 4.5 and 5.5. In some embodiments, the pH of the exchange composition is about 5.0. In some embodiments, the trehalose composition comprises between or between about 20 and 50 g/L of the antibody, such as between or between about 25 and 45 g/L, or between or between about 30 and 40 g/L. In some embodiments, the trehalose composition comprises about 35 g/L of the antibody.

Methods of the disclosure may include concentrating and depolarizing the trehalose composition. In some embodiments, the depolarizing comprises recirculating the trehalose composition at a pressure of ≤30 psig. In some embodiments, the recirculating is carried out for ≤60 minutes. In some embodiments, the recirculating is carried out for about 10 minutes. In some embodiments, the concentrating comprises using plug flow chase. In some such embodiments where the concentrating comprises using plug flow chase, the trehalose composition comprises between 27.5 g/L and 32.5 g/L, inclusive of the endpoints, of the antibody.

Methods of the disclosure include contacting a trehalose composition with a phosphate composition to produce a pH adjusted composition. In some embodiments, the phosphate composition comprises, consists essentially of, or consists of about 500 mM sodium phosphate. In some embodiments, the final concentration of the sodium phosphate in the pH adjusted composition is about 50 mM. In some embodiments the pH of the pH adjusted composition is between or between about 5.0 and 7.0, such as between or between about 5.9 and 6.3, inclusive of the endpoints. In some embodiments, the pH of the pH adjusted composition is about 6.1.

In some embodiments, the phosphate composition comprises between or between about 450 and 550 mM sodium phosphate, between or between about 500 and 520 mM sodium phosphate, or about 510 mM sodium phosphate. In some embodiments, the final concentration of sodium phosphate in the pH adjusted composition is between or between about 45 and 55 mM, such as about 51 mM. In some embodiments, the phosphate composition comprises sodium phosphate monobasic. In some embodiments, the phosphate composition comprises sodium phosphate dibasic. In some embodiments, the phosphate composition comprises both sodium phosphate monobasic and sodium phosphate dibasic. In some embodiments, the phosphate composition comprises sodium phosphate dibasic anhydrous. In some embodiments, the phosphate composition comprises sodium phosphate monobasic monohydrate. In some embodiments, the phosphate composition comprises sodium phosphate monobasic dihydrate. In some embodiments, the phosphate composition comprises trehalose. In some embodiments, the phosphate composition comprises α,α′-trehalose. In some embodiments, the phosphate composition comprises between or between about 10 and 15 g/L sodium phosphate dibasic anhydrous, such as about 12 g/L sodium phosphate dibasic anhydrous. In some embodiments, the phosphate composition comprises between or between about 50 and 75 g/L sodium phosphate monobasic monohydrate, such as about 58 g/L sodium phosphate monobasic monohydrate. In some embodiments, the phosphate composition comprises between or between about 50 and 70 g/L or between or between about 55 and 65 g/L α,α′-trehalose, such as about 60 g/L α,α′-trehalose. In some embodiments, the phosphate composition has a pH of between or between about 5.0 and 7.0, such as between or between about 5.5 and 6.0. In some embodiments, the phosphate composition has a pH of 5.74.

In some embodiments, the contacting step has a duration of between 1 second and 3600 seconds, such as between or between about 1000 and 3000, between or between about 1500 and 2500 seconds, inclusive of the endpoints, or about 1800 seconds.

Methods of the disclosure include formulating the pH adjusted composition for delivery to a subject to produce a stable antibody composition. In some embodiments, the stable antibody composition comprises ≤15% high molecular weight species (HMWS), such as ≤10%, ≤7.5%, ≤6%, ≤5%, ≤4%, ≤3%, ≤2.5%, ≤2%, ≤1.5%, or ≤1% HMWS. In some embodiments, the stable antibody composition comprises, consists essentially of, or consists of ≤6% high molecular weight species (HMWS).

In some embodiments, the formulating step comprises contacting the pH adjusted composition with a polysorbate composition. In some embodiments, the polysorbate composition comprises about 20%, about 15%, about 10%, about 5%, or about 1% (m/v) polysorbate 20. In some embodiments, the polysorbate composition comprises about 10% (m/v) polysorbate 20.

In some embodiments, the stable antibody composition has a pH of between 5.9 and 6.3, inclusive of the endpoints. In some embodiments, the stable antibody composition has a pH of 6.1. In some embodiments, the stable antibody composition has a final concentration of the antibody of between or between about 15 and 35 mg/ml, such as between or between about 20 and 30 mg/ml, inclusive of the endpoints. In some embodiments, the stable antibody composition has a final concentration of the antibody of between 22.5 mg/ml and 27.5 mg/ml, inclusive of the endpoints. In some embodiments, the stable antibody composition has a final concentration of between or between about 0.01% and 0.1% (m/v) polysorbate 20, such as between or between about 0.02% and 0.06% (m/v) polysorbate 20. In some embodiments, the stable antibody composition has a final concentration of 0.04% (m/v) polysorbate 20.

In some embodiments, the stable antibody composition has a conductivity of between or between about 2 and 6 mS/cm, such as between or between about 3 and 5 mS/cm, inclusive of the endpoints. In some embodiments, the stable antibody composition has a conductivity of between about 3.5 and about 4.5 mS/cm, inclusive of the endpoints.

In some embodiments, an expected yield of antibody in the stable antibody composition following the formulating step is ≥80%, ≥85%, or ≥90%. In some embodiments, the expected yield of antibody in the stable antibody composition following the formulating step is ≥95%.

In some embodiments, the stable antibody composition comprises ≤15%, ≤12%, ≤10%, ≤8%, ≤7%, ≤6%, or ≤5% HMWS twenty four months after completion of the formulating step. In some embodiments, the stable antibody composition comprises ≤8% HMWS twenty four months after completion of the formulating step. In some embodiments, the stable antibody composition accumulates between or between about 0.1% and 1%, 0.2% and 0.6%, or 0.3% and 0.4%, inclusive of the endpoints, of HMWS per month after completion of the formulating step. In some embodiments, the stable antibody composition accumulates between 0.25% and 0.50%, inclusive of the endpoints, of HMWS per month after completion of the formulating step.

In some embodiments, the stable antibody composition comprises ≤5, 4, 3, 2.5, 2, 1.5, 1, or 0.5%, inclusive of the endpoints, oxidation of methionine residues of the amino acid sequence of SEQ ID NO: 1. In some embodiments, the stable antibody composition comprises ≤2.5%, inclusive of the endpoints, oxidation of methionine residues of the amino acid sequence of SEQ ID NO: 1. In some embodiments, the stable antibody composition comprises ≤5, 4, 3, 2.5, 2, 1.5, 1, or 0.5%, inclusive of the endpoints, oxidation of methionine residues of the amino acid sequence of the amino acid sequence of SEQ ID NO: 2. In some embodiments, the stable antibody composition comprises ≤2.5%, inclusive of the endpoints, oxidation of methionine residues of the amino acid sequence of the amino acid sequence of SEQ ID NO: 2. In a particular embodiment, the oxidation of methionine residues of the amino acid sequence of SEQ ID NO: 1 comprises oxidation of the methionine at position 258 of SEQ ID NO: 1.

In some embodiments, the stable antibody composition is stored at −20° C.±5° C. within 10, 20, 30, 40, 50, 60, 70, 80, 90, 100, or more days following the completion of the formulating step. In some embodiments, the stable antibody composition is stored at −20° C.±5° C. within 60 days following the completion of the formulating step. In some embodiments, the stable antibody composition is stored at −20° C.±5° C. within 60 days following a date of manufacture of the stable antibody composition.

The stable antibody composition may be used for treating cancer in a subject in need thereof. In some embodiments, treatment includes administering to the subject the stable antibody composition in an amount effective to treat said cancer. In some embodiments, the subject is a human being. The cancer may be platinum-resistant recurrent epithelial ovarian cancer, fallopian tube cancer, primary peritoneal cancer, persistent cervical cancer, recurrent cervical cancer, metastatic cervical cancer, metastatic colorectal cancer, metastatic HER2 negative breast cancer, metastatic renal cell carcinoma, glioblastoma, or non-small cell lung cancer.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents, and other references mentioned herein are incorporated by reference in their entireties for all purposes. The references cited herein are not admitted to be prior art to the claimed disclosure. In the case of conflict, the present specification, including definitions, will control. In addition, the materials, methods, and examples are illustrative only and are not intended to be limiting. Other features and advantages of the disclosure will be apparent from the following detailed description and claims.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

Various terms relating to aspects of the present invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

As used herein, the terms “comprising,” “having,” and “including” encompass the more restrictive terms “consisting essentially of” and “consisting of.”

The terms subject and patient are used interchangeably, and include any animal. Subjects include mammals, including companion and farm mammals, as well as rodents, including mice, rabbits, and rats, and other rodents. Non-human primates preferred subjects. Human beings are highly preferred subjects.

The terms composition and formulation are used interchangeably. Accordingly, a formulation of the disclosure may be a composition of the disclosure and a composition of the disclosure may be a formulation of the disclosure.

It has been observed in accordance with the invention that formulations of a bevacizumab biosimilar antibody, which specifically binds to vascular endothelial growth factor, can be buffered with citrate phosphate, along with trehalose or sucrose, or buffered with acetate (instead of citrate phosphate) along with sucrose, with the buffers enhancing the thermal and colloidal stability of the antibody, even more so than formulations of bevacizumab (sold under the trade name Avastin®) currently approved for patient use. In particular, the inventive formulations demonstrated significantly lower antibody aggregation. The buffers enhance the shelf life of the antibody molecule. Accordingly, the disclosure features buffered formulations of a bevacizumab biosimilar antibody that include an aqueous carrier comprising a buffer comprising citrate phosphate, as well as trehalose or sucrose, at an acidic pH or, in the alternative, an aqueous carrier comprising a buffer comprising acetate, as well as sucrose, at an acidic pH.

The antibody specifically binds to an epitope on vascular endothelial growth factor (VEGF), and the epitope may be linear or conformational. In some aspects, the antibody comprises a heavy chain comprising the amino acid sequence of SEQ ID NO: 1, or a sequence having at least or about 80, 85, 90, 95, or 99% identity thereto. In some preferred aspects, the antibody comprises a heavy chain comprising the amino acid sequence of SEQ ID NO: 1. In some preferred aspects, the antibody comprises a light chain comprising the amino acid sequence of SEQ ID NO: 2. Preferably, the antibody comprises a heavy chain constant domain and/or a light chain constant domain. In highly preferred aspects, the antibody comprises a heavy chain comprising the amino acid sequence of SEQ ID NO: 1 and a light chain comprising the amino acid sequence of SEQ ID NO: 2. In some aspects, the antibody comprises a heavy chain variable region of the amino acid sequence of SEQ ID NO: 3 and the light chain variable region of the amino acid sequence of SEQ ID NO:4.

Bevacizumab Heavy Chain IgG1 (SEQ ID NO: 1)
EVQLVESGGG LVQPGGSLRL SCAASGYTFT NYGMNWVRQA
PGKGLEWVGW INTYTGEPTY AADFKRRFTF SLDTSKSTAY
LQMNSLRAED TAVYYCAKYP HYYGSSHWYF DVWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKK VEPKSCDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCNVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLSLS PGK
Bevacizumab Light Chain (SEQ ID NO: 2)
DIQMTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
Bevacizumab Heavy Chain Variable Region
(SEQ ID NO: 3)
EVQLVESGGG LVQPGGSLRL SCAASGYTFT NYGMNWVRQA
PGKGLEWVGW INTYTGEPTY AADFFRRFTF SLDTSKSTAY
LQMNSLRAED TAVYYCAKYP HYYGSSHWYF DVWGQGTLVT
VSS
Bevacizumab Light Chain Variable Region
(SEQ ID NO: 4)
DIQMTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKR

In some embodiments, the antibody is a full-length antibody, comprising both variable and constant regions, although in some aspects, the antibody may comprise a derivative or fragment or portion of a full-length antibody that retains the antigen-binding specificity, and also preferably retains most or all of the affinity, of the full length antibody molecule. The antibody may comprise post-translational modifications (PTMs) or moieties, which may impact antibody activity or stability. The antibody may be methylated, acetylated, glycosylated, sulfated, phosphorylated, carboxylated, and/or amidated, and may comprise other moieties that are well known in the art.

The formulation preferably comprises a therapeutically effective amount of the antibody. A therapeutically effective amount may vary, depending on the disease or condition being treated upon administration of the antibody, and/or depending on the characteristics of the subject to which the antibody is administered, such as age, gender, height, weight, state of advancement or stage of the disease or condition, the number and efficacy of previous administrations, other therapeutic agents administered to the subject, and other characteristics that are known to the practitioner or that would otherwise be taken into account in determining appropriate dosing. Preferably, a therapeutically effective amount is an amount that is effective to treat cancers such as non-squamous non-small cell lung cancer, glioblastoma, renal cell carcinoma, cervical cancer, or epithelial ovarian, fallopian tube, or primary peritoneal cancer.

The formulation may comprise from about 10 mg/ml to about 50 mg/ml of the antibody. In some aspects, the formulation comprises from about 10 mg/ml to about 40 mg/ml of the antibody. In some aspects, the formulation comprises from about 10 mg/ml to about 30 mg/ml of the antibody. In some aspects, the formulation comprises from about 20 mg/ml to about 50 mg/ml of the antibody. In some aspects, the formulation comprises from about 20 mg/ml to about 40 mg/ml of the antibody. In some aspects, the formulation comprises from about 20 mg/ml to about 30 mg/ml of the antibody. In some aspects, the formulation comprises from about 15 mg/ml to about 45 mg/ml of the antibody. In some aspects, the formulation comprises from about 15 mg/ml to about 35 mg/ml of the antibody. In some aspects, the formulation comprises from about 15 mg/ml to about 30 mg/ml of the antibody. In some aspects, the formulation comprises from about 21 mg/ml to about 29 mg/ml of the antibody. In some aspects, the formulation comprises from about 22 mg/ml to about 28 mg/ml of the antibody. In some aspects, the formulation comprises from about 23 mg/ml to about 27 mg/ml of the antibody. In some aspects, the formulation comprises from about 24 mg/ml to about 25 mg/ml of the antibody. In some aspects, the formulation comprises from about 25 mg/ml to about 30 mg/ml of the antibody. In some aspects, the formulation comprises from about 25 mg/ml to about 26 mg/ml of the antibody. In some aspects, the formulation comprises from about 25 mg/ml to about 27 mg/ml of the antibody. In some aspects, the formulation comprises from about 25 mg/ml to about 28 mg/ml of the antibody. In some aspects, the formulation comprises from about 25 mg/ml to about 29 mg/ml of the antibody. In some aspects, the formulation comprises from about 25 mg/ml to about 30 mg/ml of the antibody. In some aspects, the formulation comprises from about 24 mg/ml to about 27 mg/ml of the antibody. In some aspects, the formulation comprises from about 24 mg/ml to about 28 mg/ml of the antibody. In some aspects, the formulation comprises from about 24 mg/ml to about 29 mg/ml of the antibody. In some aspects, the formulation comprises from about 24 mg/ml to about 30 mg/ml of the antibody. In some aspects, the formulation comprises from about 25.5 mg/ml to about 26 mg/ml of the antibody. In some aspects, the formulation comprises from about 25.4 mg/ml to about 25.9 mg/ml of the antibody. In some aspects, the formulation comprises from about 25.6 mg/ml to about 25.9 mg/ml of the antibody. In some aspects, the formulation comprises from about 25.5 mg/ml to about 25.8 mg/ml of the antibody. In some aspects, the formulation comprises from about 25.5 mg/ml to about 25.7 mg/ml of the antibody. These ranges include the lower and upper amounts that define the range. In some aspects, the formulation comprises about 25 mg/ml of the antibody. In some aspects, the formulation comprises about 25.5 mg/ml of the antibody. In some aspects, the formulation comprises about 25.6 mg/ml of the antibody. In some aspects, the formulation comprises about 25.7 mg/ml of the antibody. In some aspects, the formulation comprises about 25.8 mg/ml of the antibody.

The antibody, for example, at the concentrations described or exemplified herein, is preferably formulated with a buffered aqueous carrier, and the carrier preferably comprises water. The buffered antibody formulation is preferably in liquid form, and more preferably in liquid form suitable for intravenous administration. Thus, the amount of water in the buffered formulation may vary in accordance with the desired volume of the infusion. In some preferred aspects, the buffer comprises citrate phosphate, trehalose, and a mild surfactant such as polysorbate 20, and maintains the antibody formulation at an acidic pH of from about 5.8 to about 6.0. In some alternate preferred aspects, the buffer comprises acetate, sucrose, and a mild surfactant such as polysorbate 20, and maintains the antibody formulation at an acidic pH of from about 5.6 to about 5.8. When stored in the buffered formulation, the antibody is shelf-stable under normal storage conditions.

Citrate phosphate comprises an aqueous combination of dibasic sodium phosphate dodecahydrate and citric acid monohydrate, in a pre-mixed solution comprising about 0.2 M of dibasic sodium phosphate and about 0.1 M of citric acid.

The buffer may comprise from about 10 mM to about 100 mM of citrate phosphate. In some aspects, the buffer may comprise from about 20 mM to about 90 mM of citrate phosphate. In some aspects, the buffer may comprise from about 30 mM to about 70 mM of citrate phosphate. In some aspects, the buffer may comprise from about 30 mM to about 80 mM of citrate phosphate. In some aspects, the buffer may comprise from about 40 mM to about 70 mM of citrate phosphate. In some aspects, the buffer may comprise from about 40 mM to about 60 mM of citrate phosphate. In some aspects, the buffer may comprise from about 45 mM to about 55 mM of citrate phosphate. In some aspects, the buffer may comprise from about 46 mM to about 54 mM of citrate phosphate rate. In some aspects, the buffer may comprise from about 47 mM to about 53 mM of citrate phosphate. In some aspects, the buffer may comprise from about 48 mM to about 52 mM of citrate phosphate. In some aspects, the buffer may comprise from about 49 mM to about 51 mM of citrate phosphate. In some aspects, the buffer may comprise from about 40 mM to about 50 mM of citrate phosphate. In some aspects, the buffer may comprise from about 50 mM to about 75 mM of citrate phosphate. In some aspects, the buffer may comprise from about 30 mM to about 55 mM of citrate phosphate. In some aspects, the buffer may comprise from about 40 mM to about 55 mM of citrate phosphate. In some aspects, the buffer may comprise from about 42 mM to about 52 mM of citrate phosphate. In some aspects, the buffer may comprise from about 46 mM to about 52 mM of citrate phosphate. In some aspects, the buffer may comprise from about 43 mM to about 53 mM of citrate phosphate. These ranges include the lower and upper amounts that define the range. In some aspects, the buffer comprises about 50 mM of citrate phosphate.

The citrate phosphate buffer may comprise from about 100 mM to about 200 mM of trehalose. In some aspects, the buffer may comprise from about 110 mM to about 190 mM of trehalose. In some aspects, the buffer may comprise from about 120 mM to about 180 mM of trehalose. In some aspects, the buffer may comprise from about 130 mM to about 170 mM of trehalose. In some aspects, the buffer may comprise from about 140 mM to about 170 mM of trehalose. In some aspects, the buffer may comprise from about 150 mM to about 170 mM of trehalose. In some aspects, the buffer may comprise from about 155 mM to about 165 mM of trehalose. In some aspects, the buffer may comprise from about 150 mM to about 160 mM of trehalose. In some aspects, the buffer may comprise from about 153 mM to about 164 mM of trehalose. In some aspects, the buffer may comprise from about 152 mM to about 167 mM of trehalose. In some aspects, the buffer may comprise from about 154 mM to about 164 mM of trehalose. In some aspects, the buffer may comprise from about 155 mM to about 163 mM of trehalose. In some aspects, the buffer may comprise from about 156 mM to about 162 mM of trehalose. In some aspects, the buffer may comprise from about 157 mM to about 161 mM of trehalose. In some aspects, the buffer may comprise from about 158 mM to about 160 mM of trehalose. In some aspects, the buffer may comprise from about 158.5 mM to about 158.9 mM of trehalose. In some aspects, the buffer may comprise from about 158.6 mM to about 158.8 mM of trehalose. In some aspects, the buffer may comprise from about 158 mM to about 161 mM of trehalose. In some aspects, the buffer may comprise from about 159 mM to about 161 mM of trehalose. In some aspects, the buffer may comprise from about 157 mM to about 160 mM of trehalose. In some aspects, the buffer may comprise from about 157 mM to about 159 mM of trehalose. In some aspects, the buffer may comprise from about 150 mM to about 159 mM of trehalose. In some aspects, the buffer may comprise from about 159 mM to about 160 mM of trehalose. In some aspects, the buffer may comprise from about 159 mM to about 165 mM of trehalose. These ranges include the lower and upper amounts that define the range. In some aspects, the buffer comprises about 159 mM of trehalose. In some aspects, the buffer comprises about 158.7 mM of trehalose. In some aspects, sucrose may be used in any of these concentrations in place of trehalose. Thus, for example, the citrate phosphate buffer may comprise sucrose as a stabilizer instead of trehalose.

The acetate-sucrose buffer may comprise from about 1 mM to about 30 mM of acetate. In some aspects, the buffer may comprise from about 5 mM to about 25 mM of acetate. In some aspects, the buffer may comprise from about 10 mM to about 20 mM of acetate. In some aspects, the buffer may comprise from about 11 mM to about 19 mM of acetate. In some aspects, the buffer may comprise from about 12 mM to about 18 mM of acetate. In some aspects, the buffer may comprise from about 13 mM to about 15 mM of acetate. In some aspects, the buffer may comprise from about 10 mM to about 15 mM of acetate. In some aspects, the buffer may comprise from about 12 mM to about 16 mM of acetate. In some aspects, the buffer may comprise from about 12 mM to about 15 mM of acetate. In some aspects, the buffer may comprise from about 13 mM to about 16 mM of acetate. In some aspects, the buffer may comprise from about 13 mM to about 17 mM of acetate. In some aspects, the buffer may comprise from about 14 mM to about 18 mM of acetate. In some aspects, the buffer may comprise from about 14 mM to about 16 mM of acetate. In some aspects, the buffer may comprise from about 15 mM to about 20 mM of acetate. In some aspects, the buffer may comprise from about 5 mM to about 15 mM of acetate. In some aspects, the buffer may comprise from about 11 mM to about 17 mM of acetate. In some aspects, the buffer may comprise from about 15 mM to about 16 mM of acetate. These ranges include the lower and upper amounts that define the range. In some aspects, the buffer comprises about 15 mM of acetate. Preferably, the acetate is sodium acetate trihydrate.

The acetate-sucrose or citrate phosphate-sucrose buffer may comprise from about 100 mM to about 250 mM of sucrose. In some aspects, the buffer may comprise from about 125 mM to about 225 mM of sucrose. In some aspects, the buffer may comprise from about 150 mM to about 200 mM of sucrose. In some aspects, the buffer may comprise from about 155 mM to about 195 mM of sucrose. In some aspects, the buffer may comprise from about 160 mM to about 190 mM of sucrose. In some aspects, the buffer may comprise from about 165 mM to about 185 mM of sucrose. In some aspects, the buffer may comprise from about 166 mM to about 184 mM of sucrose. In some aspects, the buffer may comprise from about 167 mM to about 183 mM of sucrose. In some aspects, the buffer may comprise from about 168 mM to about 182 mM of sucrose. In some aspects, the buffer may comprise from about 169 mM to about 181 mM of sucrose. In some aspects, the buffer may comprise from about 170 mM to about 180 mM of sucrose. In some aspects, the buffer may comprise from about 171 mM to about 179 mM of sucrose. In some aspects, the buffer may comprise from about 172 mM to about 178 mM of sucrose. In some aspects, the buffer may comprise from about 174 mM to about 177 mM of sucrose. In some aspects, the buffer may comprise from about 174 mM to about 176 mM of sucrose. In some aspects, the buffer may comprise from about 175 mM to about 175.5 mM of sucrose. In some aspects, the buffer may comprise from about 175.2 mM to about 175.4 mM of sucrose. In some aspects, the buffer may comprise from about 175 mM to about 185 mM of sucrose. In some aspects, the buffer may comprise from about 165 mM to about 175 mM of sucrose. In some aspects, the buffer may comprise from about 170 mM to about 190 mM of sucrose. In some aspects, the buffer may comprise from about 150 mM to about 175 mM of sucrose. These ranges include the lower and upper amounts that define the range. In some aspects, the buffer comprises about 175 mM of sucrose. In some aspects, the buffer comprises about 175.3 mM of sucrose.

The antibody formulation (e.g., with the citrate phosphate-trehalose or the acetate sucrose buffer) preferably comprises a non-ionic surfactant. More preferably, the non-ionic surfactant comprises polysorbate 20 (may comprise Tween® 20 brand polysorbate of Croda International Plc, Yorkshire, England). The antibody formulation, including the antibody and the aqueous buffer, preferably comprises from about 0.01% to about 0.1% (by volume) of polysorbate 20. In some aspects, the antibody formulation comprises from about 0.02% to about 0.09% (by volume) of polysorbate 20. In some aspects, the antibody formulation comprises from about 0.03% to about 0.08% (by volume) of polysorbate 20. In some aspects, the antibody formulation comprises from about 0.01% to about 0.07% (by volume) of polysorbate 20. In some aspects, the antibody formulation comprises from about 0.02% to about 0.06% (by volume) of polysorbate 20. In some aspects, the antibody formulation comprises from about 0.03% to about 0.05% (by volume) of polysorbate 20. In some aspects, the antibody formulation comprises from about 0.04% to about 0.06% (by volume) polysorbate 20. In some aspects, the antibody formulation comprises from about 0.02% to about 0.05% (by volume) of polysorbate 20. In some aspects, the antibody formulation comprises from about 0.02% to about 0.04% (by volume) of polysorbate 20. In some aspects, the antibody formulation comprises from about 0.03% to about 0.06% (by volume) of polysorbate 20. In some aspects, the antibody formulation comprises from about 0.01% to about 0.05% (by volume) of polysorbate 20. In some aspects, the antibody formulation comprises from about 0.03% to about 0.04% (by volume) of polysorbate 20. In some aspects, the antibody formulation comprises from about 0.04% to about 0.05% (by volume) of polysorbate 20. In some aspects, the antibody formulation comprises from about 0.035% to about 0.045% (by volume) of polysorbate 20. These ranges include the lower and upper amounts that define the range. In some aspects, the antibody formulation comprises about 0.04% (by volume) of polysorbate 20.

The antibody formulation (e.g., with the citrate phosphate-trehalose/sucrose or the acetate sucrose buffer) preferably is buffered to an acidic pH. The formulation preferably has a pH of from about 5.3 to about 6.5. In some aspects, the formulation has a pH of about 5.4 to about 6.4. In some preferred aspects, the formulation has a pH of about 5.4 to about 5.9. In some preferred aspects, the formulation has a pH of about 5.5 to about 5.8. In some preferred aspects, the formulation has a pH of about 5.6 to about 5.8. In some preferred aspects, the formulation has a pH of about 5.6 to about 5.9. In some aspects, the formulation has a pH of about 5.5 to about 5.3. In some preferred aspects, the formulation has a pH of about 5.6 to about 6.2. In some aspects, the formulation has a pH of about 5.7 to about 6.1. In some aspects, the formulation has a pH of about 5.8 to about 6.0. In some preferred aspects, the formulation has a pH of about 5.4 to about 5.9. In some aspects, the formulation has a pH of about 5.6 to about 5.9. In some preferred aspects, the formulation has a pH of about 5.7 to about 5.9. In some preferred aspects, the formulation has a pH of about 5.9 to about 6.1. In some aspects, the formulation has a pH of about 6.0 to about 6.2. In some aspects, the formulation has a pH of about 5.7 to about 6.0. In some preferred aspects, the formulation has a pH of from about 5.8 to about 6.1. These ranges include the lower and upper amounts that define the range. In some aspects, the formulation has a pH of about 5.8. In some aspects, the formulation has a pH of about 5.9. In some aspects, the formulation has a pH of about 6.0.

In some preferred aspects, the antibody formulation comprises from about 20 mg/ml to about 30 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 30 mM to about 70 mM of citrate phosphate, from about 150 mM to about 170 mM of trehalose, and from about 0.01% to about 0.07% (by volume) of polysorbate 20, and has a pH of from about 5.6 to about 6.0. In some aspects, the antibody formulation consists essentially of from about 20 mg/ml to about 30 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 30 mM to about 70 mM of citrate phosphate, from about 150 mM to about 170 mM of trehalose, and from about 0.01% to about 0.07% (by volume) of polysorbate 20, and has a pH of from about 5.6 to about 6.0. In some aspects, the antibody formulation consists of from about 20 mg/ml to about 30 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 30 mM to about 70 mM of citrate phosphate, from about 150 mM to about 170 mM of trehalose, and from about 0.01% to about 0.07% (by volume) of polysorbate 20, and has a pH of from about 5.6 to about 6.0. In any such embodiments, the antibody may be present in the formulation at from about 21 mg/ml to about 29 mg/ml, or from about 22 mg/ml to about 28 mg/ml, or from about 23 mg/ml to about 27 mg/ml, of from about 24 mg/ml to about 26 mg/ml, or from about 24.5 mg/ml to about 26.5 mg/ml, about 25 mg/ml, about 26 mg/ml, about 25.5 mg/ml, about 25.6 mg/ml, about 25.7 mg/ml or about 25.8 mg/ml.

In some preferred aspects, the antibody formulation comprises from about 20 mg/ml to about 30 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 40 mM to about 60 mM of citrate phosphate, from about 154 mM to about 164 mM of trehalose, and from about 0.02% to about 0.06% (by volume) of polysorbate 20, and has a pH of from about 5.6 to about 6.0, or a pH of about 5.8, or a pH of about 6.0. In some aspects, the antibody formulation consists essentially of from about 20 mg/ml to about 30 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 40 mM to about 60 mM of citrate phosphate, from about 154 mM to about 164 mM of trehalose, and from about 0.02% to about 0.06% (by volume) of polysorbate 20, and has a pH of from about 5.6 to about 6.0, or a pH of about 5.8, or a pH of about 6.0. In some aspects, the antibody formulation consists of from about 20 mg/ml to about 30 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 40 mM to about 60 mM of citrate phosphate, from about 154 mM to about 164 mM of trehalose, and from about 0.02% to about 0.06% (by volume) of polysorbate 20, and has a pH of from about 5.6 to about 6.0, or a pH of about 5.8, or a pH of about 6.0. In any such embodiments, the antibody may be present in the formulation at from about 21 mg/ml to about 29 mg/ml, or from about 22 mg/ml to about 28 mg/ml, or from about 23 mg/ml to about 27 mg/ml, of from about 24 mg/ml to about 26 mg/ml, or from about 24.5 mg/ml to about 26.5 mg/ml, about 25 mg/ml, about 26 mg/ml, about 25.5 mg/ml, about 25.6 mg/ml, about 25.7 mg/ml or about 25.8 mg/ml.

In some preferred aspects, the antibody formulation comprises from about 25 mg/ml to about 26.5 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 45 mM to about 55 mM of citrate phosphate, from about 157 mM to about 161 mM of trehalose, and from about 0.03% to about 0.05% (by volume) of polysorbate 20, and has a pH of from about 5.6 to about 6.0, or a pH of about 5.8, or a pH of about 6.0. In some aspects, the antibody formulation consists essentially of from about 25 mg/ml to about 26.5 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 45 mM to about 55 mM of citrate phosphate, from about 157 mM to about 161 mM of trehalose, and from about 0.03% to about 0.05% (by volume) of polysorbate 20, and has a pH of from about 5.6 to about 6.0, or a pH of about 5.8, or a pH of about 6.0. In some aspects, the antibody formulation consists of from about 25 mg/ml to about 26.5 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising from about 45 mM to about 55 mM of citrate phosphate, from about 157 mM to about 161 mM of trehalose, and from about 0.03% to about 0.05% (by volume) of polysorbate 20, and has a pH of from about 5.6 to about 6.0, or a pH of about 5.8, or a pH of about 6.0. In any such embodiments, the antibody may be present in the formulation at from about 25 mg/ml to about 26 mg/ml, or from about 25.5 mg/ml to about 26 mg/ml, about 25 mg/ml, about 26 mg/ml, about 25.5 mg/ml, about 25.6 mg/ml, about 25.7 mg/ml or about 25.8 mg/ml.

In some preferred aspects, the antibody formulation comprises from about 25.5 mg/ml to about 26.1 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising about 50 mM of citrate phosphate, about 159 mM of trehalose, and about 0.04% (by volume) of polysorbate 20, and has a pH of about 5.8 or about 6.0. In some aspects, the antibody formulation consists essentially of from about 25.5 mg/ml to about 26.1 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising about 50 mM of citrate phosphate, about 159 mM of trehalose, and about 0.04% (by volume) of polysorbate 20, and has a pH of about 5.8 or about 6.0. In some aspects, the antibody formulation consists of from about 25.5 mg/ml to about 26.1 mg/ml of an antibody that specifically binds to VEGF and comprises a heavy chain comprising the amino acid sequence of SEQ ID NO: 1 and a light chain comprising the amino acid sequence of SEQ ID NO: 2, a buffer comprising about 50 mM of citrate phosphate, about 159 mM of trehalose, and about 0.04% (by volume) of polysorbate 20, and has a pH of about 5.8 or about 6.0. In any such embodiments, the antibody may be present in the formulation at about 26 mg/ml, about 25.5 mg/ml, about 25.6 mg/ml, about 25.7 mg/ml or about 25.8 mg/ml.

The formulation stabilizes the antibody for improved shelf storage, particularly over a period of months to years. When stored in the formulation, the antibody maintains thermal and colloidal stability during the period of storage. For example, when stored in the formulation, the antibody is stable and exhibits minimal aggregation, flocculation, fragmentation, and denaturation, and the antibody retains its VEGF binding activity.

It is preferred that the antibody formulation be stored under refrigerated conditions, and preferably at a temperature of from about 2° C. to about 6° C., including about 2° C., about 3° C., about 4° C., about 5° C., about 6° C., about 7° C. about 8° C. The antibody formulation may be stored at such temperatures for at least about 3 months. In some aspects, the antibody formulation may be stored at such temperatures for at least about 6 months. In some aspects, the antibody formulation may be stored at such temperatures for at least about 9 months. In some aspects, the antibody formulation may be stored at such temperatures for at least about 12 months. In some aspects, the antibody formulation may be stored at such temperatures for at least about 15 months. In some aspects, the antibody formulation may be stored at such temperatures for at least about 18 months. In some aspects, the antibody formulation may be stored at such temperatures for at least about 21 months. In some aspects, the antibody formulation may be stored at such temperatures for at least about 24 months. During the storage period the antibody is stable and exhibits minimal aggregation, flocculation, fragmentation, and denaturation, and the antibody retains it VEGF binding activity such that the antibody formulation may be removed from storage, administered to a patient, and still exhibit therapeutic efficacy against the condition for which the formulation is administered.

The formulation preferably comprises about 20 mg/ml to about 30 mg/ml of antibody and, more preferably about 25 mg/ml or about 25.5 mg/ml, or about 26 mg/ml of antibody. Among this amount of antibody protein is a percentage of antibody monomers in active, native form, as well as a percentage of antibody aggregates that have reduced or no VEGF binding activity. It is highly preferred that that the formulation include a maximal amount of functional antibody monomers and a minimal amount of antibody aggregates, and structurally altered forms of the antibody with reduced binding activity and/or therapeutic efficacy (relative to the unaltered monomer). For example, the antibody formulation preferably contains at least about 85% by weight of antibody monomers, and less than about 15% by weight of antibody aggregates with reduced VEGF binding activity and/or therapeutic efficacy when stored at about 2° C. to about 6° C. for at least about six months.

In some aspects, the antibody formulation contains at least about 90% by weight of antibody monomers, and less than about 10% by weight of antibody aggregates with reduced VEGF binding activity and/or therapeutic efficacy when stored at about 2° C. to about 8° C. for at least about six months. In some aspects, the antibody formulation contains at least about 93% by weight of antibody monomers, and less than about 7% by weight of antibody aggregates with reduced VEGF binding activity and/or therapeutic efficacy when stored at about 2° C. to about 8° C. for at least about six months. In some aspects, the antibody formulation contains at least about 95% by weight of antibody monomers, and less than about 5% by weight of antibody aggregates with reduced VEGF binding activity and/or therapeutic efficacy when stored at about 2° C. to about 8° C. for at least about six months. In some aspects, the antibody formulation contains at least about 96% by weight of antibody aggregates with reduced VEGF binding activity and/or therapeutic efficacy when stored at about 2° C. to about 8° C. for at least about six months. In some aspects, the antibody formulation contains at least about 97% by weight of antibody monomers, and less than about 3% by weight of antibody aggregates with reduced VEGF binding activity and/or therapeutic efficacy when stored at about 2° C. to about 8° C. for at least about six months. In some aspects, the antibody formulation contains at least about 98% by weight of antibody monomers, and less than about 2% by weight of antibody aggregates with reduced VEGF binding activity and/or therapeutic efficacy when stored at about 2° C. to about 8° C. for at least about six months. In some aspects, the antibody formulation contains at least about 99% by weight of antibody monomers, and less than about 1% by weight of antibody aggregates with reduced VEGF binding activity and/or therapeutic efficacy when stored at about 2° C. to about 8° C. for at least about six months. The amount of antibody monomers and/or antibody aggregates may be determined according to any technique suitable in the art, including those described or exemplified herein, including any one or combination of differential light scattering (DLS), differential scanning calorimetry (DSC), size exclusion chromatography (SE-HPLC), non-reducing and reducing capillary electrophoresis SDS (NR CE-SDS and R CE-SDS), and particulate count (PC).

In some aspects, the antibody formulation contains at least about 90% by weight of antibody monomers, and less than about 10% by weight of antibody aggregates with reduced VEGF binding activity and/or therapeutic efficacy when stored at about 2° C. to about 8° C. for at least about twelve months. In some aspects, the antibody formulation contains at least about 93% by weight of antibody monomers, and less than about 7% by weight of antibody aggregates with reduced VEGF binding activity and/or therapeutic efficacy when stored at about 2° C. to about 8° C. for at least about twelve months. In some aspects, the antibody formulation contains at least about 95% by weight of antibody monomers, and less than about 5% by weight of antibody aggregates with reduced VEGF binding activity and/or therapeutic efficacy when stored at about 2° C. to about 8° C. for at least about twelve months. In some aspects, the antibody formulation contains at least about 96% by weight of antibody aggregates with reduced VEGF binding activity and/or therapeutic efficacy when stored at about 2° C. to about 8° C. for at least about twelve months. In some aspects, the antibody formulation contains at least about 97% by weight of antibody monomers, and less than about 3% by weight of antibody aggregates with reduced VEGF binding activity and/or therapeutic efficacy when stored at about 2° C. to about 8° C. for at least about twelve months. In some aspects, the antibody formulation contains at least about 98% by weight of antibody monomers, and less than about 2% by weight of antibody aggregates with reduced VEGF binding activity and/or therapeutic efficacy when stored at about 2° C. to about 8° C. for at least about twelve months. In some aspects, the antibody formulation contains at least about 99% by weight of antibody monomers, and less than about 1% by weight of antibody aggregates with reduced VEGF binding activity and/or therapeutic efficacy when stored at about 2° C. to about 8° C. for at least about twelve months. The amount of antibody monomers and/or antibody aggregates may be determined according to any technique suitable in the art, including those described or exemplified herein, including any one or combination of differential light scattering (DLS), differential scanning calorimetry (DSC), size exclusion chromatography (SE-HPLC), non-reducing and reducing capillary electrophoresis SDS (NR CE-SDS and R CE-SDS), and particulate count (PC).

In some aspects, the antibody formulation contains at least about 90% by weight of antibody monomers, and less than about 10% by weight of antibody aggregates with reduced VEGF binding activity and/or therapeutic efficacy when stored at about 2° C. to about 8° C. for at least about eighteen months. In some aspects, the antibody formulation contains at least about 93% by weight of antibody monomers, and less than about 7% by weight of antibody aggregates with reduced VEGF binding activity and/or therapeutic efficacy when stored at about 2° C. to about 8° C. for at least about eighteen months. In some aspects, the antibody formulation contains at least about 95% by weight of antibody monomers, and less than about 5% by weight of antibody aggregates with reduced VEGF binding activity and/or therapeutic efficacy when stored at about 2° C. to about 8° C. for at least about eighteen months. In some aspects, the antibody formulation contains at least about 96% by weight of antibody aggregates with reduced VEGF binding activity and/or therapeutic efficacy when stored at about 2° C. to about 8° C. for at least about eighteen months. In some aspects, the antibody formulation contains at least about 97% by weight of antibody monomers, and less than about 3% by weight of antibody aggregates with reduced VEGF binding activity and/or therapeutic efficacy when stored at about 2° C. to about 8° C. for at least about eighteen months. In some aspects, the antibody formulation contains at least about 98% by weight of antibody monomers, and less than about 2% by weight of antibody aggregates with reduced VEGF binding activity and/or therapeutic efficacy when stored at about 2° C. to about 8° C. for at least about eighteen months. In some aspects, the antibody formulation contains at least about 99% by weight of antibody monomers, and less than about 1% by weight of antibody aggregates with reduced VEGF binding activity and/or therapeutic efficacy when stored at about 2° C. to about 8° C. for at least about eighteen months. The amount of antibody monomers and/or antibody aggregates may be determined according to any technique suitable in the art, including those described or exemplified herein, including any one or combination of differential light scattering (DLS), differential scanning calorimetry (DSC), size exclusion chromatography (SE-HPLC), non-reducing and reducing capillary electrophoresis SDS (NR CE-SDS and R CE-SDS), and particulate count (PC).

In some aspects, the antibody formulation contains at least about 90% by weight of antibody monomers, and less than about 10% by weight of antibody aggregates with reduced VEGF binding activity and/or therapeutic efficacy when stored at about 2° C. to about 8° C. for at least about twenty-four months. In some aspects, the antibody formulation contains at least about 93% by weight of antibody monomers, and less than about 7% by weight of antibody aggregates with reduced VEGF binding activity and/or therapeutic efficacy when stored at about 2° C. to about 8° C. for at least about twenty-four months. In some aspects, the antibody formulation contains at least about 95% by weight of antibody monomers, and less than about 5% by weight of antibody aggregates with reduced VEGF binding activity and/or therapeutic efficacy when stored at about 2° C. to about 8° C. for at least about twenty-four months. In some aspects, the antibody formulation contains at least about 96% by weight of antibody aggregates with reduced VEGF binding activity and/or therapeutic efficacy when stored at about 2° C. to about 8° C. for at least about twenty-four months. In some aspects, the antibody formulation contains at least about 97% by weight of antibody monomers, and less than about 3% by weight of antibody aggregates with reduced VEGF binding activity and/or therapeutic efficacy when stored at about 2° C. to about 8° C. for at least about twenty-four months. In some aspects, the antibody formulation contains at least about 98% by weight of antibody monomers, and less than about 2% by weight of antibody aggregates with reduced VEGF binding activity and/or therapeutic efficacy when stored at about 2° C. to about 8° C. for at least about twenty-four months. In some aspects, the antibody formulation contains at least about 99% by weight of antibody monomers, and less than about 1% by weight of antibody aggregates with reduced VEGF binding activity and/or therapeutic efficacy when stored at about 2° C. to about 8° C. for at least about twenty-four months. The amount of antibody monomers and/or antibody aggregates may be determined according to any technique suitable in the art, including those described or exemplified herein, including any one or combination of differential light scattering (DLS), differential scanning calorimetry (DSC), size exclusion chromatography (SE-HPLC), non-reducing and reducing capillary electrophoresis SDS (NR CE-SDS and R CE-SDS), and particulate count (PC).

### Methods of Producing Stable Antibody Compositions

The methods of the disclosure produce stable antibody compositions. These stable antibody compositions may be referred to as ONS-5010. In some embodiments, ONS-5010 comprises an antibody biosimilar, sodium phosphate monobasic, sodium phosphate dibasic, α,α′-trehalose, and polysorbate 20. In some embodiments, the stable antibody compositions contain minimal amounts of HMWS, accumulate HMWS at a slower rate over time, and/or maintain low amounts of HMWS during long-term storage (e.g., as compared to other antibody compositions).

Generally, HMWS contribute to the formation of covalent, non-reversible aggregates that diminish binding of an antibody, e.g., bevacizumab, to its epitope on VEGF, thus reducing the therapeutic efficacy of the antibody. Typically, limiting the amount of HMWS reduces this problem and enhances the activity of the antibody. In addition, regulatory standards limit the allowable amount of HMWS in antibody formulations of bevacizumab to less than or equal to 8% after twenty-four months. Therefore, it is desirable to limit the amount of HMWS in stable antibody compositions, to slow the accumulation of HMWS over time, and to maintain low amounts of HMWS during long-term storage.

In some embodiments, diafiltration is a component of the penultimate step in the manufacturing process of the stable antibody composition (e.g., antibody biosimilar) of the instant disclosure. Generally, pH values at or above 6.2 during diafiltration increase the HMWS in formulations of antibody, e.g., bevacizumab, causing greater accumulation of HMWS over time. Accordingly, the present disclosure provides methods of diafiltration that decrease HMWS by diafiltering a concentrated composition into an exchange solution comprising trehalose and, following diafiltration, rapidly adjusting the pH of the composition by addition of a phosphate composition. In some embodiments, the addition of the phosphate composition prevents generation and accumulation of HMWS during manufacture and storage of the stable antibody compositions.

The present disclosure provides a method of producing a stable antibody composition. The method includes ultrafiltering a starting composition to produce a concentrated composition. In some aspects, the starting composition comprises between or between about 4 mg/ml and 6 mg/ml, inclusive of the endpoints, of an antibody. In some aspects, the starting composition has a pH of about 4.7 to about 5.3, inclusive of the endpoints. In certain aspects, the pH of the starting composition is about 5.0.

In some aspects, methods of the disclosure produce a stable antibody composition comprising ultrafiltering a starting composition. In some aspects, methods of the disclosure employ a starting composition comprising, consisting essentially of, or consisting of an antibody and a starting buffer composition. In some embodiments, the starting composition comprises, consists essentially of, or consists of between or between about 4 mg/ml and 6 mg/ml of an antibody comprising a heavy chain comprising the amino acid sequence of SEQ ID NO:1 and a light chain comprising the amino acid sequence of SEQ ID NO: 2.

In some aspects, the starting buffer composition has a pH of about 5.0. An exemplary starting buffer composition of the disclosure includes, but is not limited to, acetate. In some aspects, the starting buffer composition has a conductivity of between or between about 20 and 30 mS/cm, such as about 25 mS/cm. In some aspects, the starting buffer composition has a conductivity of 25 mS/cm. In some aspects embodiments, the starting buffer composition comprises one or more monovalent or bivalent metal ions at a concentration that does not decrease the stability of the antibody compared to a composition that does not comprise one or more monovalent or bivalent metal ions. Exemplary monovalent or bivalent (or divalent) metal ions include, but are not limited to, hydrogen (H), lithium (Li), sodium (Na), magnesium (Mg), potassium (K), calcium (Ca), manganese (Mn), iron (Fe), cobalt (Co) and zinc (Zn).

In some aspects, methods of the disclosure may be referred to as ultrafiltration/diafiltration (UF/DF). Generally, membrane size, material, and load may affect protein adsorption or retention during UF/DF. In some aspects, methods of the disclosure use polyethersulfone membranes. In some aspects, methods of the disclosure use membranes with a 30 kD molecular weight pore size. Membrane load, which is generally a factor of the desired process time and the permeate flux (volume of permeate over time), may affect the quality of product obtained from UF/DF. The permeate flux is typically influenced by the feed rate (LMH), retentate pressure/Trans-membrane pressure, and the viscosity of the material. In some embodiments, the membrane bears a load of ≤1000 g/m², ≤750 g/m², ≤500 g/m², or ≤250 g/m². In some embodiments, the membrane bears a load of ≤about 500 g/m² to ≤about 100 g/m². In some embodiments, the membrane bears a load of ≤about 300 g/m². In some aspects, feed flow rates of 5450 LMH are used in the methods of the disclosure. In some aspects, a feed flow rate of 375 LMH is used in the methods of the disclosure. In some aspects, a retentate pressure of ≤25 psi is used in the methods of the disclosure. In some aspects, a retentate pressure of 5 psi is used in the methods of the disclosure. In some aspects, a TMP of ≤20 psig is used in the methods of the disclosure. In some aspects, a TMP of 15 psig is used in the methods of the disclosure.

In some aspects, compositions of the disclosure (e.g., the starting composition, concentrated composition, trehalose composition) may be ultrafiltered prior to diafiltration. In some aspects, compositions of the disclosure may be concentrated by ultrafiltration prior to diafiltration. In some aspects, ultrafiltration produces a concentrated composition comprising between or between about 30 and 40 mg/ml, inclusive of the endpoints, of the antibody.

Diafiltration may be used to remove, or decrease the concentration of, salts or solvents in compositions or formulations of the present disclosure. Diafiltration may be continuous or discontinuous. Regenerated cellulose membranes or polyethersulfone membranes can be employed for diafiltration. Generally, these membranes, or cassettes, have a wide pH and temperature range. Membranes used for diafiltration are typically available in variety of molecular weight cutoffs including 1 kDa, 30 kD, and 100 kD. In some aspects, the methods of the disclosure employ membranes with a 30 kD molecular weight pore size. These membranes may be equilibrated prior to diafiltration. In some aspects, the membranes are equilibrated with sodium acetate. In some aspects, about 25 mM sodium acetate is used to equilibrate the membranes. In some aspects, the membranes are equilibrated with sodium chloride. In some aspects, about 240 mM sodium chloride is used to equilibrate the membranes. In some aspects, 237 mM sodium chloride is used. In some aspects, the membranes are equilibrated with a trehalose solution. In some aspects, the trehalose solution comprises about 6% trehalose. In some aspects, the trehalose solution comprises trehalose in water. Membranes may also be equilibrated to a desired pH and conductivity. In some aspects, the membranes are equilibrated to a pH of between or between about 4.5 and 5.5, such as about 5.0. In some aspects, the membranes are equilibrated to a conductivity of between or between about 20 and 30 mS/cm, such as about 25 mS/cm.

In some aspects, diafiltration comprises exchanging the starting buffer composition of the concentrated composition with an exchange solution. In some embodiments, the exchange solution may be aqueous. In some aspects, the exchange solution comprises trehalose. In some embodiments, the exchange solution comprises from about 4% to about 8% trehalose (w/v), inclusive of the endpoints. In certain aspects, the exchange solution comprises 6% trehalose (w/v) in water. In some aspects, the trehalose is α,α′-trehalose. In some aspects, the exchange solution comprises polysorbate, including, as a non-limiting example, polysorbate 20. In some aspects, a polysorbate composition comprising about 10% of polysorbate is used to produce a final concentration of about 0.03% to about 0.05% polysorbate 20 in the exchange composition and/or the trehalose composition. In some aspects, the exchange solution and/or trehalose composition comprises a final concentration of 0.04% polysorbate. In some aspects, ≥5 diavolumes of the exchange solution may be used during diafiltration. As a non-limiting example, the amount of the exchange solution used may be 5.1, 5.2, 5.3, 5.4, 5.5, 5.6, 5.7, 5.8, 5.9, 6.0, 6.5, 7.0, 7.5, or 8.0 diavolumes.

In some aspects, the trehalose composition comprises between or between about 30 g/L and 40 g/L, inclusive of the endpoints, of the antibody. In some aspects, the trehalose composition comprises about 35 g/L of the antibody. In some embodiments, the trehalose composition comprises 35 g/L of the antibody.

In some aspects, the trehalose composition has a pH of about 4.7 to about 5.3, inclusive of the endpoints. In certain aspects, the pH of the trehalose composition is about 5.0.

Additional process steps may be employed to increase recovery or improve quality of products of UF/DF. For example, product loss may result from loss in the permeate due to product that has passed through the membrane, bound to the membrane and cannot be desorbed prior to recovery, or product that is otherwise lost in the system. Recovery procedures may use air, buffer, or gravity assist. As a non-limiting example, buffer recirculation may be used to concentrate the product and improve recovery. Buffer recirculation methods may also be used to depolarize membranes and improve product mixing. In some embodiments, a recirculation pressure of ≤30 psig is used. In some embodiments, recirculation time may be ≤60 minutes. In some embodiments, recirculation time is about 10 minutes. A plug flow rinse or chase may be used to recover product that is lost in the equipment or system. This procedure can be used to flush the product (protein, e.g. antibody) from the system. In some aspects, the volume of 6% α,α-trehalose used in plug flow rinse or chase may be selected to produce a protein concentration of about 30 g/L. In some aspects, the volume used in plug flow rinse or chase may be selected to produce a protein concentration of about 28.5 to about 31.5 g/L, inclusive of the endpoints.

In some embodiments, the method of the present disclosure also includes contacting the trehalose composition with a phosphate composition to produce a pH adjusted composition.

In some aspects, the contacting step has a duration of between or between about 1 second and 3600 seconds, inclusive of the endpoints. In some aspects, the contacting step has a duration of between or between about 1500 and 2100 seconds, inclusive of the endpoints. In some aspects, the contacting step has a duration of about 1800 seconds. Methods known in the art may be used to increase the rate at which contacting occurs. Exemplary methods may include, but are not limited to, the application of heat, pressure, or agitation to the trehalose composition. In some embodiments, heat, pressure, or agitation may be applied to the phosphate composition.

In some aspects, the phosphate composition has a pH of about 5.5 to about 5.9, inclusive of the endpoints. In certain aspects, the phosphate composition has a pH of about 5.7. In some such aspects, the phosphate composition has a pH of 5.74.

In some embodiments, the phosphate composition of the present disclosure comprises sodium phosphate. In some aspects, the phosphate composition comprises between or between about 450 mM and 550 mM, inclusive of the endpoints, sodium phosphate. In some aspects, the phosphate composition comprises about 500 mM sodium phosphate. In some such aspects, the phosphate composition comprises 510 mM.

In some aspects, the sodium phosphate is sodium phosphate monobasic. In some aspects, the sodium phosphate is sodium phosphate dibasic. In some embodiments, the sodium phosphate comprises sodium phosphate monobasic and sodium phosphate dibasic. In some aspects, the phosphate composition comprises between or between about 50 and 60 g/L, inclusive of the endpoints, of sodium phosphate monobasic. In some aspects, the phosphate composition comprises about 55 g/L sodium phosphate monobasic. In some embodiments, the phosphate composition comprises 58 g/L sodium phosphate monobasic. In some aspects, the phosphate composition comprises between or between about 10 and 20 g/L, inclusive of the endpoints, of sodium phosphate dibasic. In some aspects, the phosphate composition comprises about 15 g/L sodium phosphate dibasic. In some embodiments, the phosphate composition comprises 12 g/L sodium phosphate dibasic.

In some aspects, the phosphate composition comprises between or between about 40 and 80 g/L α,α′-trehalose, such as between or between about 50 and 70 g/L α,α′-trehalose. In some aspects, the phosphate composition comprises about 60 g/L α,α′-trehalose.

In some aspects, the final concentration of sodium phosphate in the pH adjusted composition is between about 40 mM and about 60 mM, inclusive of the endpoints. In some aspects, the final concentration of the sodium phosphate in the pH adjusted composition is between 45 mM and 55 mM, inclusive of the endpoints. In some aspects, the final concentration of the sodium phosphate in the pH adjusted composition is about 50 mM. In some aspects, the final concentration of the sodium phosphate in the pH adjusted composition is or is about 51 mM.

In some aspects, the pH of the pH adjusted composition is between about 5.9 and about 6.3, inclusive of the endpoints. In some aspects, the pH of the pH adjusted composition is between 5.9 and 6.3, inclusive of the endpoints. In some aspects, the pH of the pH adjusted composition is or is about 6.2.

In some embodiments, the method of the present disclosure includes formulating the pH adjusted composition for delivery to a subject to produce a stable antibody composition. In some aspects, the formulating step comprises adding aqueous solution (phosphate and/or polysorbate solution). In some aspects, the formulating step comprises adding a non-ionic surfactant (polysorbate 20).

In some aspects, the pH adjusted composition comprises about 3.0 to about 3.5% HMWS, inclusive of the endpoints. In some aspects, the pH adjusted composition comprises about 3.20, about 3.25, about 3.30, about 3.35, about 3.40, about 3.45, or about 3.50% HMWS. In some aspects, the pH adjusted composition comprises 3.20, 3.25, 3.30, 3.35, 3.40, 3.45, or 3.50% HMWS.

In some embodiments, the method of the present disclosure includes formulating the pH adjusted composition for delivery to a subject to produce a stable antibody composition. In some aspects, formulating comprises contacting the pH adjusted composition with a polysorbate composition.

In some aspects, the polysorbate composition comprises polysorbate 20.

In some aspects, the polysorbate composition comprises about 10% of polysorbate. In some embodiments, the polysorbate composition is used to produce a final concentration of about 0.03% to about 0.05% polysorbate 20 in the stable antibody composition. In some aspects, the stable antibody composition comprises a final concentration of 0.04% polysorbate.

In some aspects, the present disclosure provides a stable antibody composition comprising 25 g/L of an antibody comprising a heavy chain comprising the amino acid sequence of SEQ ID NO:1 and a light chain comprising the amino acid sequence of SEQ ID NO: 2, 5.8 g/L sodium phosphate monobasic monohydrate, 1.2 g/L sodium phosphate dibasic anhydrous, 60.0 g/L α,α′-trehalose dehydrate, and 0.04% (v/v) polysorbate 20. In some embodiments, the stable antibody composition comprises ≤6% HMWS.

In some aspects, the stable antibody composition has a final concentration of antibody of between or between about 22.5 g/L and 27.5 g/L, inclusive of the endpoints. In certain aspects, the stable antibody composition has a final concentration of about 25 g/L of the antibody. In certain aspects, the stable antibody composition has a final concentration of about 0.04% polysorbate 20. In certain aspects, the stable antibody composition has a final concentration of 0.04% polysorbate 20. In some embodiments, among the antibody comprised by the stable antibody composition is a percentage of antibody monomers in active, native form, as well as a percentage of antibody fragments, antibody aggregates, and denatured or partially denatured antibodies that have reduced or no tumor necrosis binding activity. In some embodiments, the stable antibody composition includes a maximal amount of functional antibody monomers and a minimal amount of antibody fragments, aggregates, and structurally altered forms of the antibody with reduced binding activity and/or therapeutic efficacy (relative to the unaltered monomer).

In some aspects, the stable antibody composition comprises about 3.0 to about 6.0% HMWS, inclusive of the endpoints. In some aspects, the stable antibody composition comprises about 3.20, about 3.25, about 3.30, about 3.35, about 3.40, about 3.45, about 3.50, about 4.00, about, 4.50, or about 6.00% HMWS.

In some aspects, the stable antibody composition accumulates between 0.25% and 0.50%, inclusive of the endpoints, of HMWS per month after the formulating step. In some aspects, the stable antibody composition maintains ≤15%, ≤12%, ≤10%, ≤8%, ≤7%, ≤6%, or ≤5% HMWS for at least twelve, at least sixteen, at least eighteen, at least twenty four, at least thirty, or at least thirty six months. In some aspects, the stable antibody composition comprises ≤8% HMWS more than twenty-four months after completion of the formulating step.

In some aspects, the stable antibody composition comprises between 0.5% and 2.5%, inclusive of the endpoints, oxidation of methionine residues of the amino acid sequence of SEQ ID NO: 1 or the amino acid sequence of SEQ ID NO: 2. In some aspects, the stable antibody composition comprises ≤2.5% oxidation of methionine residues of the amino acid sequence of SEQ ID NO: 1 or the amino acid sequence of SEQ ID NO: 2. In some aspects, the oxidation of methionine residues of the amino acid sequence of SEQ ID NO: 1 comprises oxidation of the methionine at position 258 of SEQ ID NO: 1.

In some aspects, the stable antibody composition has a pH of about 5.9 to about 6.3, inclusive of the endpoints. In certain aspects, the stable antibody composition has a pH of about 6.1. In some embodiments, the stable antibody composition has a pH of 6.1.

In some aspects, the stable antibody composition is stored at −20° C.±5° C. within 60 days following the completion of the formulating step or following a date of manufacture of the stable antibody composition. The stable antibody composition may be stored at −20° C.±5° C. within 30 days following the completion of the formulating step or following a date of manufacture of the stable antibody composition. The stable antibody composition may be stored at −20° C.±5° C. within 15 days following the completion of the formulating step or following a date of manufacture of the stable antibody composition. In some embodiments, compositions of the disclosure, e.g., the stable antibody composition, may be stored at such temperatures for at least about 3 months, at least about 6 months, at least about 9 months, at least about 12 months, at least about 15 months, at least about 18 months, at least about 21 months, at least about 24 months or any minimal number of months in between. In some embodiments, during the storage period, the antibody is stable and exhibits minimal accumulation of HMWS, such that the stable antibody composition may be removed from storage, administered to a patient, and still exhibit therapeutic efficacy against the condition for which the stable antibody composition is administered.

### Methods of Treatment

In some embodiments, compositions and formulations of the present disclosure comprise a therapeutically effective amount of the antibody. A therapeutically effective amount may vary, depending on the disease or condition being treated upon administration of the antibody, and/or depending on the characteristics of the subject to which the antibody is administered, such as age, gender, height, weight, state of advancement or stage of the disease or condition, the number and efficacy of previous administrations, other therapeutic agents administered to the subject, and other characteristics that are known to the practitioner or that would otherwise be taken into account in determining appropriate dosing. Typically, a therapeutically effective amount is an amount that is effective to treat cancers such as non-squamous non-small cell lung cancer, glioblastoma, renal cell carcinoma, cervical cancer, or epithelial ovarian, fallopian tube, or primary peritoneal cancer. In some aspects, compositions of the present disclosure may be used to treat colon cancer, lung cancer, glioblastoma, rectal cancer, brain tumors, and renal-cell carcinoma.

In some aspects, compositions of the present disclosure may be used to treat eye conditions or disorders, including, but not limited to those of the retina, sclera, vitreous, lens, pupil, iris, cornea, choroid, optic nerve, retinal vasculature, ciliary body, or angle of the eye. In some embodiments, the angle of the eye comprises the trabecular meshwork and associated structures. In some aspects, compositions of the present disclosure may be used to treat eye conditions or disorders wherein vascular endothelial growth factor (VEGF) is upregulated, dysregulated, or hyperactive. In some aspects, compositions of the present disclosure may be used to treat eye conditions or disorders, including, but not limited to, age-related macular degeneration, macular edema, diabetic macular edema (DME), retinopathy, diabetic retinopathy, myopic degeneration, idiopathic choroidal neovascularization, inflammatory choroidal neovascularization, retinal neovascularization, polyploidal choroidal vasculopathy, eye neovascularization, branch retinal vein occlusion (BRVO), central retinal vein occlusion, central serous chorioretinopathy, retinitis, retinitis pigmentosa, stargardt disease, usher syndrome, retinal degeneration, endophthalmitis, familial exudative vitreoretinopathy, idiopathic juxtafoveal telangiectasis, lattice degeneration, macular hole, persistent fetal vasculature, retinal artery occlusion, and retinoblastoma. In some aspects, compositions of the present disclosure may be used to treat eye conditions or disorders, including, but not limited to, age-related macular degeneration, wet age-related macular degeneration, and neovascular age-related macular degeneration.

In some aspects, methods of treating wet age-related macular degeneration with stable antibody compositions of the disclosure comprise inhibiting, preventing, or reducing vascular growth in the eye. In some aspects, methods of treating wet age-related macular degeneration with stable antibody compositions of the disclosure comprise inhibiting, preventing or reducing vascularization of the eye.

Stable antibody compositions of the disclosure may be administered through any appropriate route including, but not limited to, oral routes, intravenous routes, intramuscular routes, topically, subcutaneously, suprachoroidally, via eye drop, and direct absorption through mucous membrane tissues. In some aspects, stable antibody compositions of the disclosure may be administered as a solution for intravenous infusion. In some aspects, stable antibody compositions of the disclosure may be administered as an intravitreal injection. In some aspects, stable antibody compositions of the disclosure may be administered as an intravitreal infusion.

In some aspects, stable antibody compositions of the disclosure may be administered every 2 weeks. In some aspects, stable antibody compositions of the disclosure may be administered every 15 days. In some aspects, stable antibody compositions of the disclosure may be administered twice a month. It is possible that limiting the % of HMWS in compositions of an antibody would reduce the frequency of dose administration necessary to achieve therapeutic efficacy. Methods of the disclosure may produce compositions that are administered every 15-30 days. Methods of the disclosure may produce compositions that are administered about every 20 days. Methods of the disclosure may produce compositions that are administered about every three, four, five, or six weeks. Methods of the disclosure may produce compositions that are administered once a month or once every two months. In some aspects, stable antibody compositions of disclosure may be administered for 52 weeks. In some aspects, stable antibody compositions of the disclosure may be administered for about 50 weeks. In some aspects, stable antibody compositions of the disclosure may be administered for 4 weeks, 8 weeks, 16 weeks, 24 weeks, 36 weeks or 48 weeks. In some aspects, stable antibody compositions may be administered four, five, six, seven, eight, nine, 10, 12, 14, 16, 18, 20, or 25 times. It is possible that limiting the amount of % HMWS in stable antibody compositions reduces the amount, duration, or frequency of dose administration necessary to achieve therapeutic efficacy.

In some aspects, stable antibody compositions of the disclosure have an approximate half-life of 11-50 days. In some aspects, stable antibody compositions of the disclosure have an approximate half-life of 20 days. It is possible that limiting the amount of % HMWS in stable antibody compositions increases their half-life. Methods of the disclosure may produce compositions with a half-life 1, 2, 3, 4, 5, 10, 15, 20, or more days longer than antibody compositions produced by conventional methods.

Suitable compositions may contain antibody isoforms or combinations thereof along with one or more pharmaceutically acceptable carriers and/or pharmaceutically acceptable excipients.

The invention also features methods for treating a tumor in a subject in need thereof by administering a therapeutically effective amount of any of the antibody formulations described or exemplified herein. Preferably, the antibody formulations are used in methods for treating cancers such as platinum-resistant recurrent epithelial ovarian, fallopian tube, or primary peritoneal cancer, persistent, recurrent, or metastatic cervical cancer, metastatic colorectal cancer, metastatic HER2 negative breast cancer, metastatic renal cell carcinoma, glioblastoma, or non-small cell lung cancer (NSCLC). Therapeutic efficacy is attained, for example, by the bevacizumab antibody present in the administered formulation. Administration of the antibody formulation may be according to any suitable route, preferably by injection, and more preferably by intravenous injection. Administration may be carried out under the direction or supervision of a medical practitioner.

The invention also features methods for treating an eye condition or disorder in a subject in need thereof by administering a therapeutically effective amount of any of the antibody formulations described or exemplified herein. Preferably, the antibody formulations are used in methods for treating eye conditions or disorders, including, but not limited to those of the retina, sclera, vitreous, lens, pupil, iris, cornea, choroid, optic nerve, retinal vasculature, ciliary body, or angle of the eye (including the trabecular meshwork and associated structures). In some aspects, compositions of the present disclosure may be used to treat eye conditions or disorders wherein vascular endothelial growth factor (VEGF) is upregulated, dysregulated, or hyperactive. In some aspects, compositions of the present disclosure may be used to treat eye conditions or disorders, including, but not limited to, age-related macular degeneration, macular edema, diabetic macular edema (DME), retinopathy, diabetic retinopathy, myopic degeneration, idiopathic choroidal neovascularization, inflammatory choroidal neovascularization, retinal neovascularization, polyploidal choroidal vasculopathy, eye neovascularization, branch retinal vein occlusion (BRVO), central retinal vein occlusion, central serous chorioretinopathy, retinitis, retinitis pigmentosa, stargardt disease, usher syndrome, retinal degeneration, endophthalmitis, familial exudative vitreoretinopathy, idiopathic juxtafoveal telangiectasis, lattice degeneration, macular hole, persistent fetal vasculature, retinal artery occlusion, and retinoblastoma. In some aspects, compositions of the present disclosure may be used to treat eye conditions or disorders, including, but not limited to, age-related macular degeneration, wet age-related macular degeneration, and neovascular age-related macular degeneration.

The antibody formulations described and exemplified herein may be for use as a medicament. The antibody formulations described and exemplified herein may be for use in the manufacture of a medicament for the treatment of one or more of a cancer such as platinum-resistant recurrent epithelial ovarian, fallopian tube, or primary peritoneal cancer, persistent, recurrent, or metastatic cervical cancer, metastatic colorectal cancer, metastatic HER2 negative breast cancer, metastatic renal cell carcinoma, glioblastoma, or non-small cell lung cancer (NSCLC). The formulations may be for use in the treatment of platinum-resistant recurrent epithelial ovarian, fallopian tube, or primary peritoneal cancer. The formulations may be for use in the treatment of persistent, recurrent, or metastatic cervical cancer. The formulations may be for use in the treatment of metastatic colorectal cancer. The formulations may be for use in the treatment of metastatic HER2 negative breast cancer. The formulations may be for use in the treatment of metastatic renal cell carcinoma. The formulations may be for use in the treatment of glioblastoma. The formulations may be for use in the treatment of non-small cell lung cancer (NSCLC). The antibody formulations described and exemplified herein may be for use in the manufacture of a medicament for the treatment of one or more of an eye condition or disorder, including, but not limited to those of the retina, sclera, vitreous, lens, pupil, iris, cornea, choroid, optic nerve, retinal vasculature, ciliary body, or angle of the eye (including the trabecular meshwork and associated structures). In some aspects, formulations of the present disclosure may be used to treat eye conditions or disorders wherein vascular endothelial growth factor (VEGF) is upregulated, dysregulated, or hyperactive. In some aspects, formulations of the present disclosure may be used to treat eye conditions or disorders, including, but not limited to, age-related macular degeneration, macular edema, diabetic macular edema (DME), retinopathy, diabetic retinopathy, myopic degeneration, idiopathic choroidal neovascularization, inflammatory choroidal neovascularization, retinal neovascularization, polyploidal choroidal vasculopathy, eye neovascularization, branch retinal vein occlusion (BRVO), central retinal vein occlusion, central serous chorioretinopathy, retinitis, retinitis pigmentosa, stargardt disease, usher syndrome, retinal degeneration, endophthalmitis, familial exudative vitreoretinopathy, idiopathic juxtafoveal telangiectasis, lattice degeneration, macular hole, persistent fetal vasculature, retinal artery occlusion, and retinoblastoma. In some aspects, formulations of the present disclosure may be used to treat eye conditions or disorders, including, but not limited to, age-related macular degeneration, wet age-related macular degeneration, and neovascular age-related macular degeneration.

The invention also features kits. The kits may be used, for example, to practice any of the methods described or exemplified herein. In some aspects, a kit comprises any antibody formulation described or exemplified herein, and instructions for using the antibody formulation in any of the methods or uses described or exemplified herein. The kit may comprise a device for injecting the antibody formulation into a subject, including but not limited to a syringe and needle, or catheter.

The instructions included with the kit may include instructions for administering the antibody formulation in a method for treating platinum-resistant recurrent epithelial ovarian, fallopian tube, or primary peritoneal cancer, including instructions for injecting the antibody formulation into a platinum-resistant recurrent epithelial ovarian, fallopian tube, or primary peritoneal cancer patient in need thereof. In some aspects, the instructions included with the kit may include instructions for administering the antibody formulation in a method for treating persistent, recurrent, or metastatic cervical cancer, including instructions for injecting the antibody formulation into a persistent, recurrent, or metastatic cervical cancer patient in need thereof. In some aspects, the instructions included with the kit may include instructions for administering the antibody formulation in a method for treating metastatic colorectal cancer, including instructions for injecting the antibody formulation into a metastatic colorectal cancer patient in need thereof. In some aspects, the instructions included with the kit may include instructions for administering the antibody formulation in a method for treating metastatic HER2 negative breast cancer, including instructions for injecting the antibody formulation into a metastatic HER2 negative breast cancer patient in need thereof. In some aspects, the instructions included with the kit may include instructions for administering the antibody formulation in a method for treating metastatic renal cell carcinoma, including instructions for injecting the antibody formulation into a metastatic renal cell carcinoma patient in need thereof. In some aspects, the instructions included with the kit may include instructions for administering the antibody formulation in a method for treating glioblastoma, including instructions for injecting the antibody formulation into a glioblastoma patient in need thereof. In some aspects, the instructions included with the kit may include instructions for administering the antibody formulation in a method for treating non-small cell lung cancer (NSCLC), including instructions for injecting the antibody formulation into a non-small cell lung cancer (NSCLC) patient in need thereof. The instructions included with the kit may include instructions for administering the antibody formulation in a method for treating an eye condition, including, but not limited to those of the retina, sclera, vitreous, lens, pupil, iris, cornea, choroid, optic nerve, retinal vasculature, ciliary body, or angle of the eye (including the trabecular meshwork and associated structures). In some aspects, compositions of the present disclosure may be used to treat eye conditions or disorders wherein vascular endothelial growth factor (VEGF) is upregulated, dysregulated, or hyperactive. In some aspects, the instructions included with the kit may include instructions for administering the antibody formulation in a method for treating eye conditions or disorders, including, but not limited to, age-related macular degeneration, macular edema, diabetic macular edema (DME), retinopathy, diabetic retinopathy, myopic degeneration, idiopathic choroidal neovascularization, inflammatory choroidal neovascularization, retinal neovascularization, polyploidal choroidal vasculopathy, eye neovascularization, branch retinal vein occlusion (BRVO), central retinal vein occlusion, central serous chorioretinopathy, retinitis, retinitis pigmentosa, stargardt disease, usher syndrome, retinal degeneration, endophthalmitis, familial exudative vitreoretinopathy, idiopathic juxtafoveal telangiectasis, lattice degeneration, macular hole, persistent fetal vasculature, retinal artery occlusion, and retinoblastoma. In some aspects, the instructions included with the kit may include instructions for administering the antibody formulation in a method for treating eye conditions or disorders, including, but not limited to, age-related macular degeneration, wet age-related macular degeneration, and neovascular age-related macular degeneration.

The following examples are provided to describe the invention in greater detail. They are intended to illustrate, not to limit, the invention.

### Example 1—Materials and Methods

Introduction. Antibody ONS-5010 represents a biosimilar of bevacizumab, and has been reformulated for enhanced storage stability. It is believed that the buffered formulation may, at the very least, reduce aggregation of the antibody during long-term storage. It is believed that the buffered formulation may reduce both the non-covalent and covalent dimerization of the bevacizumab molecule. Bevacizumab marketed as Avastin® (Genentech, Inc.) is formulated in a sodium phosphate buffer, including trehalose as a stabilizer, and including a mild surfactant and an acidic pH of 6.2. The experimental approach described below included development work to reformulate bevacizumab for enhanced colloidal stability. Significant enhancement in stability and, particularly with respect to a reduction in aggregation, was attained by changing the buffer and the pH.

Dynamic Light Scattering (DLS). The DLS testing method used a Wyatt DynaPro™ Plate Reader to provide information on protein size distribution and overall colloidal stability in solution. Hydrodynamic radius provided information on the presence of aggregation and confirmation of the molecule's structure in solution. DLS testing provided an orthogonal measure of size distribution in solution under non-denaturing conditions.

Differential Scanning Calorimetry (DSC). Differential scanning calorimetry measured the melting transitions for the protein and, thus, provided information on protein thermal stability in solution. Calorimetry was performed using a GE VP Capillary DSC system. The protein was heated from 25° C. to 95° C. at an optimized scan rate allowing the melting transitions (Tm) to occur while the protein is unfolding. A buffer control was heated alongside the sample and used to calculate melting temperatures and transitions. The DSC profile was typical of antibodies and demonstrated that the protein folded into distinct domains.

Size Exclusion Chromatography (SE-HPLC). SE-HPLC was used to monitor antibody size variant distribution. The SE-HPLC testing method separates proteins based on size. Species eluting before the monomer peak were aggregates (HMWS) and peaks eluting after the monomer peak were degradants (LMWS).

Species were separated using a TSK3000SWxl 7.8 mm×300 mm column (Tosoh Bioscience Cat #08541), with a flow rate of 0.5 mL/min and a run time of 30 minutes; column at ambient temperature. The mobile phase comprised 0.2M potassium phosphate and 0.25M potassium chloride and a pH of 6.2. There were two forms of sample injection—neat injection 10 uL @ 25 mg/mL and dilute injection 100 uL @ 0.5 mg/mL (Neat injection measures the total aggregates including reversible aggregates, dilute injection measures the dimers primarily of covalent nature). Dilute samples were diluted with the mobile phase A (0.2M potassium phosphate, 0.25M potassium chloride, pH 6.2) to 0.5 mg/mL.

Samples were incubated for 24 hours prior to analysis at 30° C. The autosampler temperature was maintained at 30° C. for the entire duration of the run. Data were analyzed at 280 nm.

Cation Exchange Chromatography (CEX). Bevacizumab samples were diluted in mobile phase A and digested with carboxypeptidase B. Species were separated using a cation-exchange HPLC column. A gradient was performed with mobile phase A and mobile phase B using a flow rate of 0.5 mL/minute. Column temperature was maintained at 40° C. and samples were maintained at 2-8° C. Data was analyzed at 280 nm.

Particulate Content (Fluid Imaging). The Fluid Imaging (FI) system is an integrated system for rapidly analyzing particles in a moving fluid. The system automatically counts, images, and analyzes the particles or cells in a sample or a continuous flow. In the FI system, the sample is drawn into the flow chamber by a pump. Using the laser in AutoImage Mode, the FI system monitored the light scatter of the passing particles. The camera was set to capture images synchronously at a user defined interval. The scatter detection values were then saved by VisualSpreadsheet (in addition to all other particle properties and the image). The computer and digital signal processor work together to initiate, retrieve and process images of the field of view.

Osmolality. An Osmometer was used to measure the osmolality of buffer and protein solutions by means of freezing-point measurement. It utilized high-precision thermisters to sense the sample temperature, to control the degree of super cooling and freeze induction, and to measure the freezing point of the sample. Sample requirement was 20 μL per measurement.

Intrinsic Fluorescence. Intrinsic Fluorescence Spectroscopy is a non-invasive biophysical characterization method that provides information on the tertiary structure of the protein. This method measured the degree of unfolding of the protein structure. Intensity and maximum wavelength of a protein sample (for example tryptophan emission) were determined on the fluorescence spectrometer. Test 600 μL of 0.1 mg/mL protein solution per replicate. Emission scan: Excitation at 295 nm, start at 310 nm end at 450 nm.

HUVEC Cell Based VEGF Neutralization Assay. The primary mechanism of action of the anti-angiogenesis monoclonal antibody bevacizumab is to bind to VEGF and prevent binding to its cognate receptor. In this way, bevacizumab neutralizes the ability of VEGF to induce endothelial cell proliferation; therefore, potency of an anti-VEGF antibody can be quantified by its ability to inhibit VEGF-induced proliferation of cells. In the HUVEC cell-based potency assay, fixed concentrations of VEGF are incubated with serially diluted drug. Bevacizumab binds to VEGF in a dose dependent manner, making VEGF unavailable for other binding interactions. This drug-VEGF cocktail is then added to HUVEC cells seeded in multi-well plates and further incubated for continued proliferation. During incubation, HUVEC cells proliferate in a VEGF concentration-dependent manner. At low drug concentrations, more VEGF is available and therefore proliferation is high and vice versa. Antibody dose-dependent inhibition of HUVEC cell proliferation is assessed by quantifying the number of viable cells at the end of incubation. The VEGF neutralization assay is a relative assay in which the potency of samples is measured relative to a reference standard. The assay consists of three independent assay plates. In each plate, the cell viability data of standard and samples are fit to 4P logistic models to generate sigmoidal curves with independent curve parameters using statistical software; standard and sample curve parameters are compared to assess curve parallelism and when deemed parallel, the relative potency of test articles is calculated. The final reported value is an average of three independent values that are within acceptable variability.

VEGF Binding Immunoassay. The primary mechanism of action of the anti-angiogenesis monoclonal antibody bevacizumab is to bind to VEGF and prevent binding to its cognate receptor, thereby inhibiting VEGF mediated mitogenic effects on vascular endothelial cells. This neutralization of VEGF by bevacizumab inhibits the angiogenesis process, which in turn suppresses tumor survival and progression. Therefore, potency of an anti-VEGF antibody can be quantified by measuring its binding to VEGF in an ELISA. In this assay, a fixed concentration of VEGF is first coated on multi-well plates. After blocking non-specific binding sites, the immobilized VEGF is reacted with serially diluted Reference standard and Test samples. The unbound antibody is washed away and the wells are incubated with horseradish peroxidase (HRP) conjugated anti-kappa light chain antibody which binds to the VEGF-Antibody complexes. Next, the unbound secondary antibody is washed away and the wells are incubated with 3,3′,5,5′-Tetramethylbenzidine (TMB) HRP substrate to produce a colored product. The color development is quenched by adding phosphoric acid and the absorbance values are read. The optical density (O.D.) values obtained are directly proportional to the amount of sample bound to VEGF. The VEGF binding assay is a relative assay in which the potency of samples is measured relative to a reference standard. The assay consists of two independent assay plates. In each plate, the O.D. data of standard and samples are fit to 4P logistic models to generate sigmoidal curves with independent curve parameters using statistical software; standard and sample curve parameters are compared to assess curve parallelism and when deemed parallel, the relative potency of test articles is calculated. The final reported value is an average of two independent values that are within acceptable variability.

### Example 2—Effect of Buffer, Stabilizers, and pH on Bevacizumab Conformational and Colloidal Stability

Initial experiments evaluated the buffer components for the conformational and colloidal stability of bevacizumab. It was determined that citrate, phosphate, and acetate buffers are ideal for stability of bevacizumab. Moreover, individually these buffers exhibited a protective effect towards aggregation of bevacizumab that is induced by heating or shaking related stress. Further experiments evaluated if a combination of these buffers (citrate, phosphate and acetate) exhibited superior stabilizing effects. A citrate phosphate buffer produced significantly lower aggregates (including covalent type dimers) and lower charge species relative to the sodium phosphate buffer in the bevacizumab match composition (matched to the formulation of the commercially available Avastin® formulation).

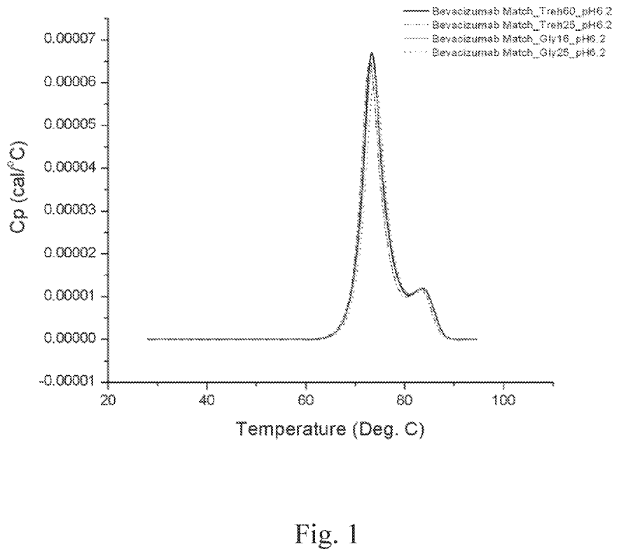

The effect of the trehalose stabilizer in a 50 mM sodium phosphate buffer was compared with alternative stabilizers, including sucrose, sorbitol, mannitol, and glycine. Conformational stability of the antibody in the different stabilized buffer composition was then assessed by DSC (FIG. 1). These data are presented in Table 1, and show that all of the stabilizers tested were equal to or better than trehalose.

TABLE 1
Alternative conformational stabilizing
agents in sodium phosphate buffer.

| | | Final | | |
|---|---|---|---|---|
| Buffer | All conditions: 50 mM | Buffer | | |
| Conditions | sodium phosphate | pH | Tₘ₁ | Tₘ₂ |
| 1 | Trehalose 60 mg/mL (match) | 6.20 | 73.3 | 83.5 |
| 2 | Trehalose 25 mg/mL | 6.26 | 73.0 | 83.0 |
| 3 | Sucrose 25 mg/mL | 6.13 | 72.9 | 82.9 |
| 4 | Sucrose 60 mg/mL | 6.11 | 73.4 | 83.4 |
| 5 | Sorbitol 25 mg/mL | 6.19 | 73.1 | 82.9 |
| 6 | Sorbitol 60 mg/mL | 6.12 | 73.6 | 83.5 |
| 7 | Mannitol 25 mg/mL | 6.19 | 73.0 | 83.2 |
| 8 | Mannitol 60 mg/mL | 6.05 | 73.7 | 83.4 |
| 9 | Glycine 16 mg/mL | 6.11 | 73.5 | 83.3 |
| 10 | Glycine 25 mg/mL | 6.05 | 73.9 | 83.6 |

The alternative stabilizers (sucrose, sorbitol, mannitol, and glycine) were next used with the citrate phosphate buffer, and the conformational stability of the bevacizumab antibody was assessed by DSC. The data are presented in Table 2, and show that the alternative stabilizers in a citrate phosphate buffer were equal to or better than the bevacizumab match formulation.

TABLE 2
Alternative conformational stabilizing agents
in citrate phosphate (C/P) buffer.

| Buffer | | | | |
|---|---|---|---|---|
| Condition | Sample | pH | Tm1 | Tm2 |
| 1 | Bevacizumab | 6.20 | 73.3 | 83.5 |
| | match: 50 mM | | | |
| | sodium phosphate, | | | |
| | Trehalose 60 mg/mL | | | |
| 2 | 50 mM C/P sucrose | 6.15 | 73.0 | 83.0 |
| | 60 gm/mL | | | |
| 3 | 50 mM C/P sorbitol | 6.14 | 73.3 | 83.3 |
| | 60 mg/mL | | | |
| 4 | 50 mM C/P mannitol | 6.11 | 73.3 | 83.2 |
| | 60 mg/mL | | | |
| 5 | 50 mM C/P glycine | 6.1 | 73.5 | 83.6 |
| | 25 mg/mL | | | |

Using the citrate phosphate buffer with trehalose as the stabilizing agent, the effect of pH on the conformational stability of the antibody (bevacizumab) was assessed. DSC was used to measure the antibody stability. The data, presented in Table 3, show that the unfolding temperatures for Bevacizumab as measured in each formulation compositions are comparable to that observed for the Bevacizumab match composition, but only at pH of greater than 5.6 (5.6, 5.8, 6.0, 6.2). At lower pH (particularly 5.0), an early unfolding event takes place at lower temperature of about 65° C., thus making such lower pH (below 5.6) unsuitable for formulation of bevacizumab.

TABLE 3
Effect of pH on bevacizumab thermal/conformational stability
in a 50 mM citrate or 50 mM citrate phosphate buffer.

| | Final | | | |
|---|---|---|---|---|
| | Buffer | | | |
| Sample | pH | Tm1 | Tm2 | Tm3 |
| Bevacizumab Match | 6.20 | 73.4 | 83.5 | |
| 35 mM Cit Treh 60 mg/mL pH 5.8 | 5.79 | 72.3 | 83.0 | |
| 50 mM Cit Treh 60 mg/mL pH 5.0 | 4.99 | 65.3 | 71.1 | 79.6 |
| 50 mM Cit Treh 60 mg/mL pH 5.2 | 5.13 | 71.5 | 80.9 | |
| 50 mM Cit Treh 60 mg/mL pH 5.4 | 5.32 | 71.9 | 82.0 | |
| 50 mM Cit Treh 60 mg/mL pH 5.6 | 5.51 | 72.1 | 82.4 | |
| 50 mM Cit Treh 60 mg/mL pH 5.8 | 5.70 | 72.2 | 82.8 | |
| 50 mM Cit Treh 60 mg/mL pH 6.0 | 5.93 | 72.3 | 82.8 | |
| 50 mM Cit Treh 60 mg/mL pH 6.2 | 6.13 | 72.4 | 83.0 | |
| 50 mM C/P Treh 60 mg/mL pH 5.0 | 5.08 | 72.2 | 81.2 | |
| 50 mM C/P Treh 60 mg/mL pH 5.2 | 5.25 | 72.5 | 81.9 | |
| 50 mM C/P Treh 60 mg/mL pH 5.4 | 5.43 | 72.8 | 82.3 | |
| 50 mM C/P Treh 60 mg/mL pH 5.6 | 5.66 | 72.8 | 83.1 | |
| 50 mM C/P Treh 60 mg/mL pH 5.8 | 5.92 | 72.9 | 83.1 | |
| 50 mM C/P Treh 60 mg/mL pH 6.0 | 6.13 | 73.0 | 83.2 | |
| 50 mM C/P Treh 60 mg/mL pH 6.2 | 6.26 | 73.1 | 83.4 | |

Parallel experiments evaluated acetate as the buffering agent. Acetate was assessed at concentrations of 5 mM, 15 mM, and 25 mM, with variable pH (Tables 4 and 5). These experiments compared sucrose (60 mg/m) and trehalose (60 mg/ml) as the stabilizing agent. Stability of the bevacizumab molecule in each composition was then assessed by DSC. The data are shown in Tables 4 and 5. It was observed that increasing the molarity of acetate lowered the Tm, and increasing the pH also lowered the Tm (Tables 4 and 5). Enhanced conformational stability was shown for pH 5.6 and 5.8 (Table 5).

TABLE 4
Conformational stability of bevacizumab in
acetate trehalose buffered formulations.

| | Final | | |
|---|---|---|---|
| Sample | pH | Tm1 | Tm2 |
| Bevacizumab Match 50 mM Phosphate pH 6.2 | 6.20 | 73.4 | 83.5 |
| 5 mM Acetate 159 mM Treh. pH 5.6 | 5.51 | 74.2 | 83.3 |
| 15 mM Acetate 159 mM Treh. pH 5.6 | 5.52 | 73.9 | 83.3 |
| 25 mM Acetate 159 mM Treh. pH 5.6 | 5.56 | 73.8 | 83.0 |
| 5 mM Acetate 159 mM Treh. pH 5.8 | 5.78 | 74.1 | 83.5 |
| 15 mM Acetate 159 mM Treh. pH 5.8 | 5.77 | 74.0 | 83.3 |
| 25 mM Acetate 159 mM Treh. pH 5.8 | 5.76 | 73.8 | 83.3 |
| 5 mM Acetate 159 mM Treh. pH 6.0 | 5.93 | 74.1 | 83.6 |
| 15 mM Acetate 159 mM Treh. pH 6.0 | 5.91 | 73.9 | 83.4 |
| 25 mM Acetate 159 mM Treh. pH 6.0 | 5.97 | 73.8 | 83.7 |
| 5 mM Acetate 159 mM Treh. pH 6.2 | 6.11 | 74.1 | 83.8 |
| 15 mM Acetate 159 mM Treh. pH 6.2 | 6.15 | 73.8 | 83.8 |
| 25 mM Acetate 159 mM Treh. pH 6.2 | 6.14 | 73.6 | 83.8 |

TABLE 5
Conformational stability of bevacizumab
in acetate sucrose buffered formulations.

| | Actual | Final pH | | |
|---|---|---|---|---|
| | Buffer | @ 0.5 | | |
| Sample | pH | mg/mL | Tm1 | Tm2 |
| Bevacizumab Match | 6.20 | 6.20 | 73.4 | 83.7 |
| 50 mM Phosphate pH 6.2 | | | | |
| 5 mM Acetate | 5.57 | 5.72 | 74.1 | 83.4 |
| 60 mg/mL Suc pH 5.6 | | | | |
| 15 mM Acetate | 5.52 | 5.60 | 73.9 | 83.0 |
| 60 mg/mL Suc pH 5.6 | | | | |
| 5 mM Acetate | 5.76 | 5.92 | 74.1 | 83.4 |
| 60 mg/mL Suc pH 5.8 | | | | |
| 15 mM Acetate | 5.73 | 5.80 | 73.9 | 83.3 |
| 60 mg/mL Suc pH 5.8 | | | | |

### Example 3—Storage Stability in Citrate Phosphate Buffered Trehalose and Acetate Sucrose Formulations

Four buffered formulations were selected to assess long-term storage stability of the bevacizumab molecule over 18 months. These formulations were run in parallel with the bevacizumab match/reference formulation (condition 1). Storage conditions were as follows: antibody at ˜25 mg/ml (neat) or diluted; storage at 5° C., 30° C., or 37° C.; shaking at 150 RPM at room temperature; and freeze/thaw (20° C. to room temperature, for three cycles). The formulations tested are listed below:

### Condition 1: Bevacizumab (Avastin®) Match

### 50 mM Sodium Phosphate

### 159 mM Trehalose

0.04% polysorbate 20
pH 6.20
Q.S. with Sterile water for injection

### Condition 2: Bevacizumab Citrate Phosphate. pH 5.8

### 50 mM Citrate Phosphate

### 159 mM Trehalose

### 0.04% Polysorbate 20

pH 5.80
Q.S. with Sterile Water for injection

### Condition 3: Bevacizumab Citrate Phosphate. pH 6.0

### 50 mM Citrate Phosphate

### 159 mM Trehalose

### 0.04% Polysorbate 20

pH 6.0
Q.S. with Sterile Water for injection

### Condition 4: Bevacizumab Acetate, pH 5.6

### 15 mM Acetate

### 175 mM Sucrose

### 0.04% Polysorbate 20

pH 5.60
Q.S. with Sterile Water for injection

### Condition 5: Bevacizumab Acetate, pH 5.8

### 15 mM Acetate

### 175 mM Sucrose

### 0.04% Polysorbate 20

pH 5.80
Q.S. with Sterile Water for injection

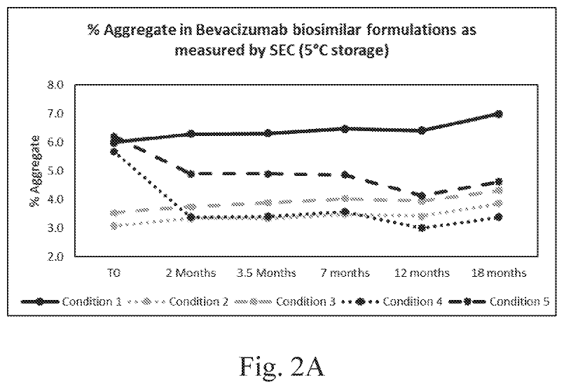

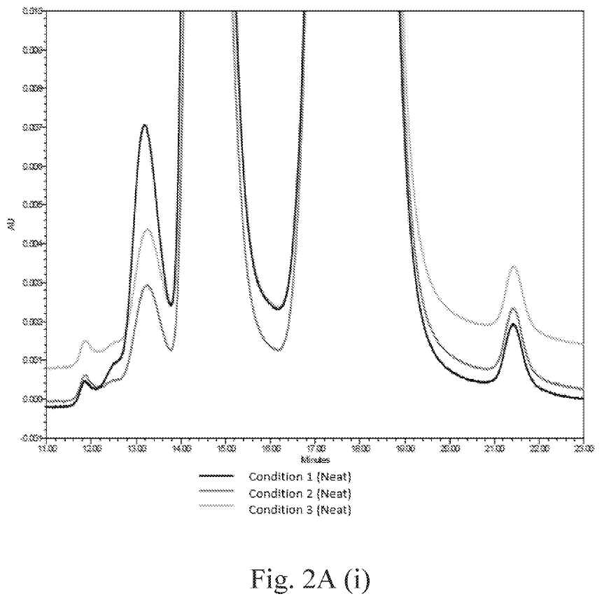

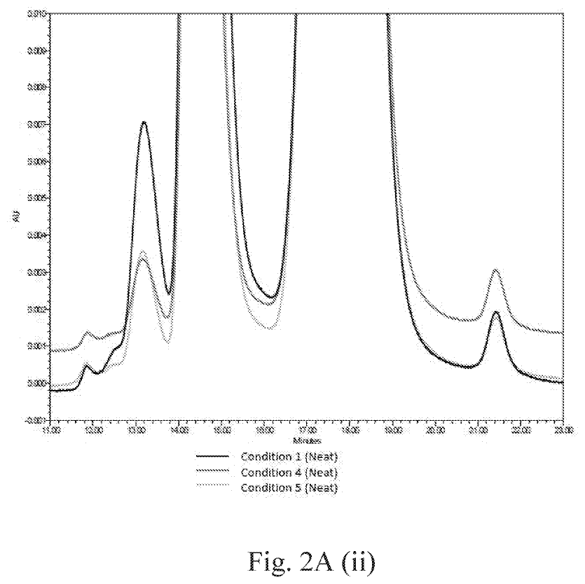

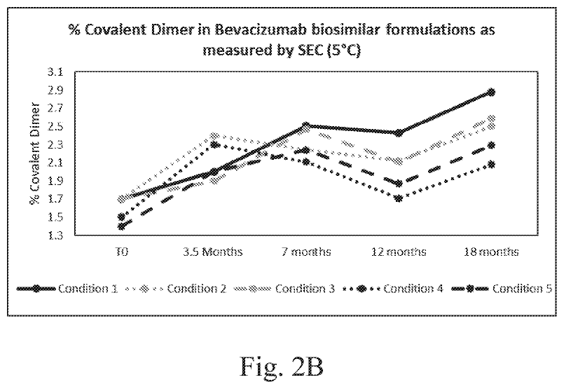

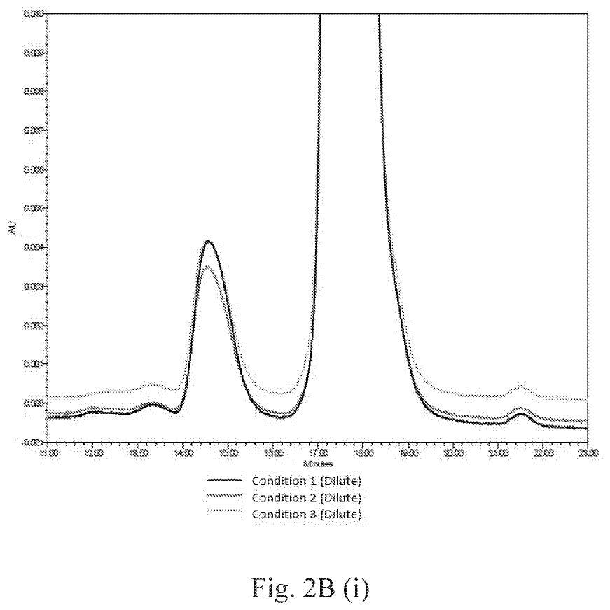

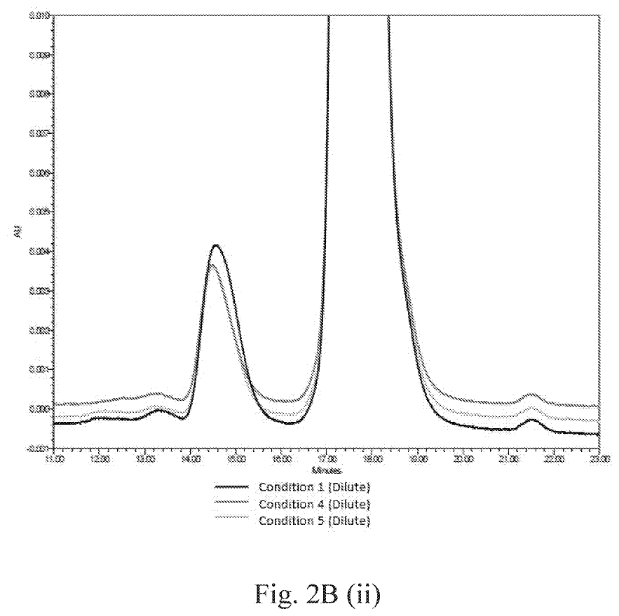

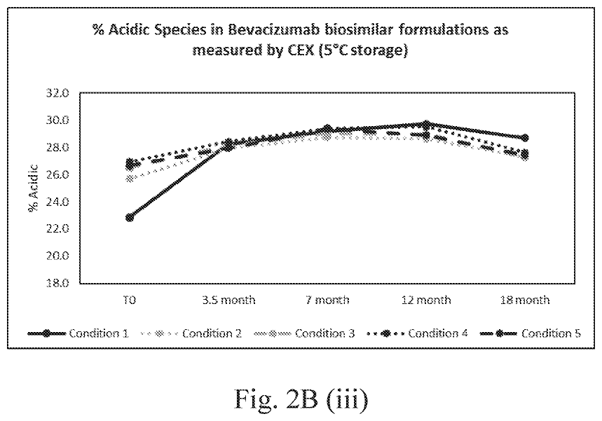

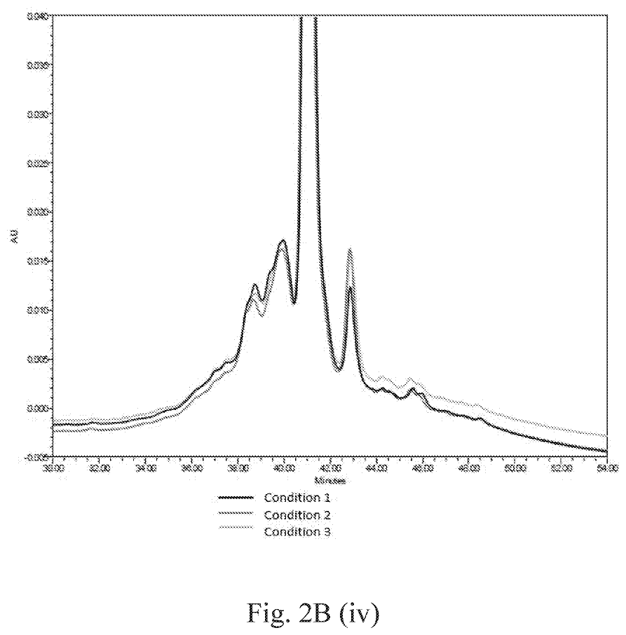

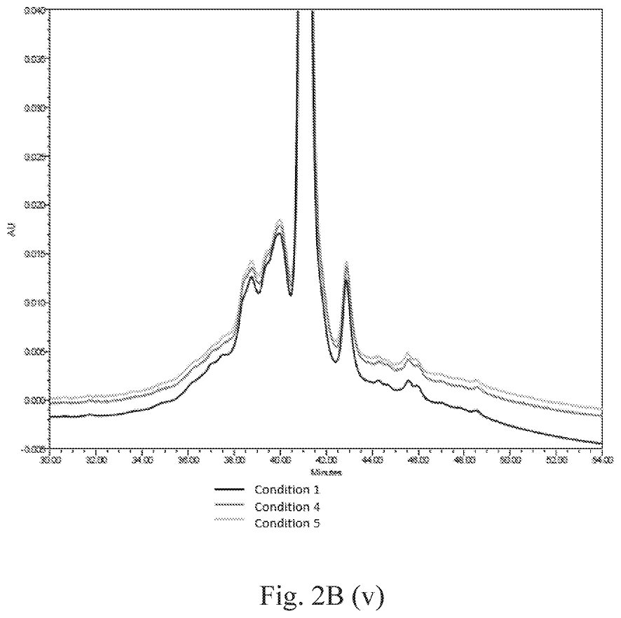

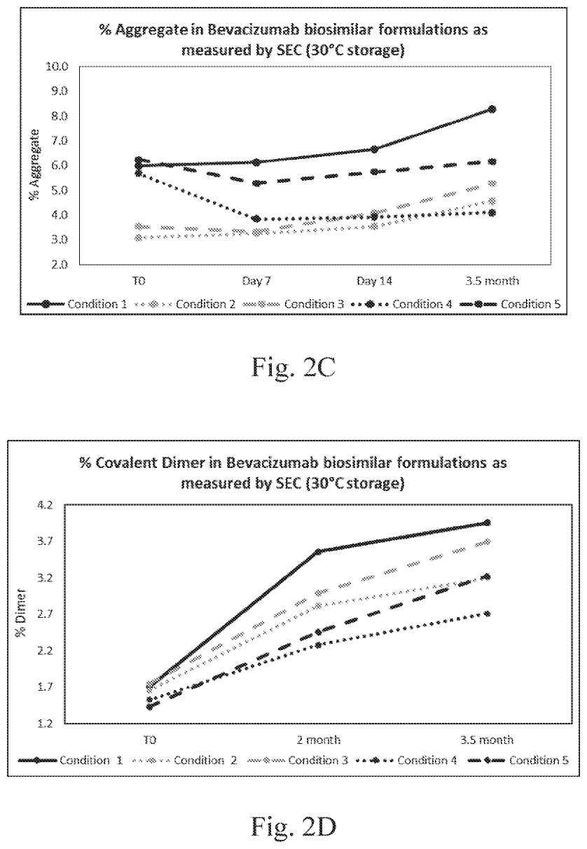

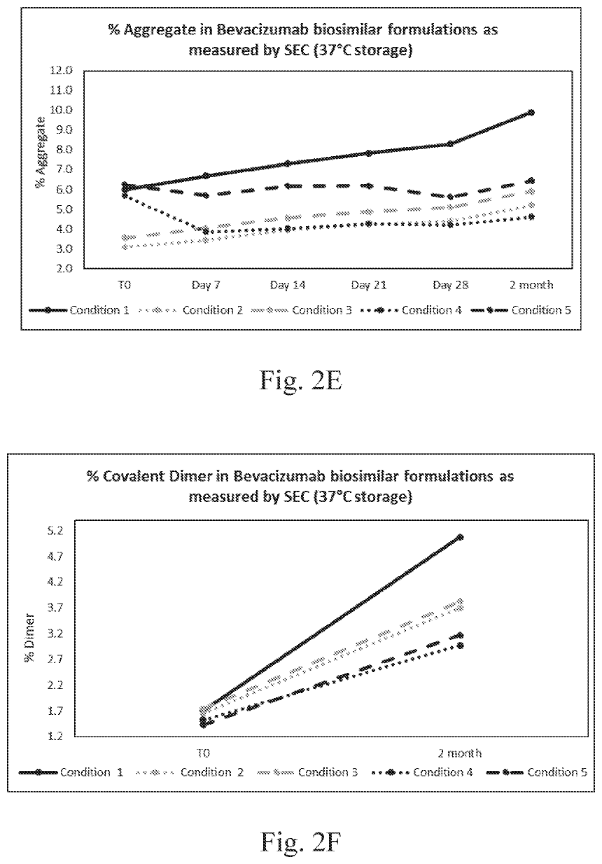

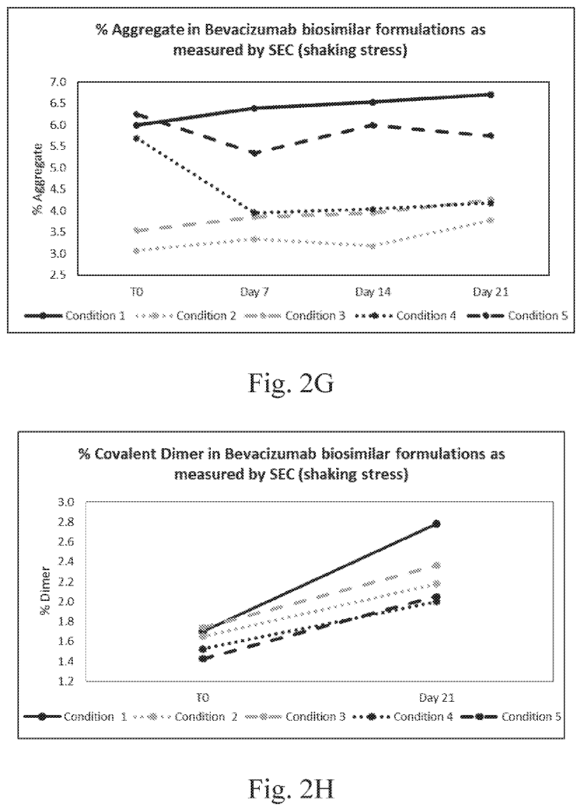

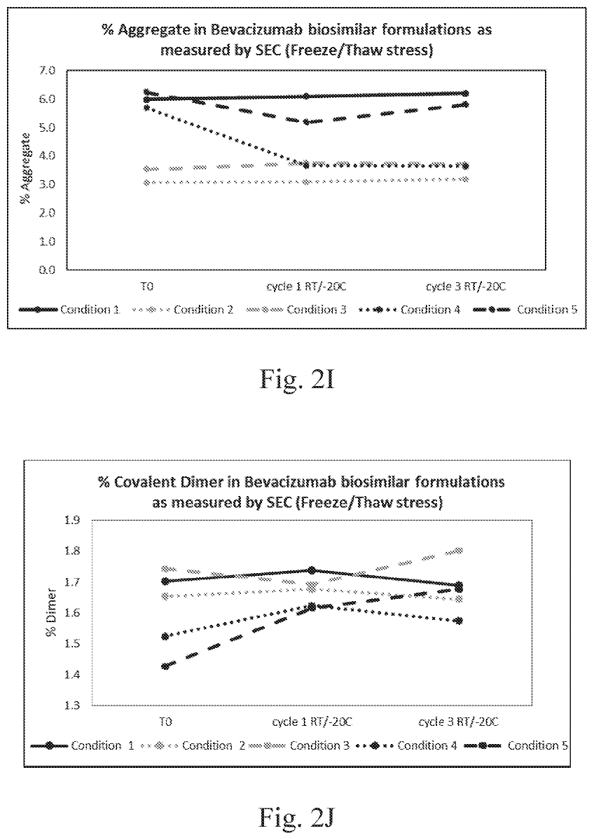

Stability of the antibody under each storage condition was tested by a battery of routine analytical and extended characterization assays, including but not limited, to size exclusion chromatography (SEC), cation exchange chromatography (CEX), CE-SDS, HUVEC cell based VEGF neutralization assay, VEGF binding immunoassay and particulate count (PC). Size exclusion chromatography was used to assess the percentage of antibody monomers, the percentage of total aggregates (covalent and non-covalent), and the percentage of degradants. The comparative stability of both formulation types were assessed along with the bevacizumab (Avastin®) reference/match composition. Samples on long term storage stability stored at 5° C.±3° C. over 18 months, indicate that both the citrate phosphate based compositions (Conditions 2 and 3) and the acetate buffer based compositions (Conditions 4 and 5) are more stable than Avastin® match composition (FIG. 2A; FIG. 2A (i); FIG. 2A (ii); Table 6). FIG. 2B, FIG. 2B (i), FIG. 2B (ii) and Table 7 indicate the measured covalent dimers is present in all five bevacizumab biosimilar compositions, however Conditions 2, 3, 4 and 5 have lower covalent dimers than those present in the bevacizumab match composition (condition 1) (Table 7).

TABLE 6
Total aggregates in bevacizumab biosimilar formulations (long term
stability at 5° C.) as measured by SE-HPLC (neat injection).

| Formulation | | | | | |
|---|---|---|---|---|---|
| Composition | % Aggregates | | | | |
| Time | Condition | Condition | Condition | Condition | Condition |
| (at 5° C.) | 1 | 2 | 3 | 4 | 5 |
| T2 | 6.0 | 3.1 | 3.5 | 5.7 | 6.2 |
| 2 Months | 6.3 | 3.4 | 3.8 | 3.4 | 4.9 |
| 3.5 Months | 6.3 | 3.3 | 3.9 | 3.4 | 4.9 |
| 7 Months | 6.5 | 3.5 | 4.0 | 3.6 | 4.9 |
| 12 Months | 6.4 | 3.4 | 4.0 | 3.0 | 4.1 |
| 18 Months | 7.0 | 3.9 | 4.3 | 3.4 | 4.6 |

TABLE 7
Covalent dimers in bevacizumab biosimilar formulations (long-
term stability) as measured by SE-HPLC (dilute injection).

| Formulation | | | | | |
|---|---|---|---|---|---|
| Composition | % Covalent Dimer | | | | |
| Time | Condition | Condition | Condition | Condition | Condition |
| (at 5° C.) | 1 | 2 | 3 | 4 | 5 |
| T2 | 1.7 | 1.7 | 1.7 | 1.5 | 1.4 |
| 3.5 Months | 2.0 | 2.4 | 1.9 | 2.3 | 2.0 |
| 7 Months | 2.5 | 2 2 | 2.5 | 2.1 | 2.2 |
| 12 Months | 2.4 | 2.1 | 2.1 | 1.7 | 1.9 |
| 18 Months | 2.9 | 2.5 | 2.6 | 2.1 | 2.3 |

Acidic charged species as measured by cation exchange chromatography (CEX) were also tested for all samples during the 18-month long storage stability study (Table 7 (i); FIG. 2B (iii); FIG. 2B (iv) and FIG. 2B (v)). Across all 5 compositions, the charged species particularly the acidic charged species did not vary significantly over the 18 months of storage at 5° C.

TABLE 7 (i)
% Acidic Species in bevacizumab biosimilar formulations
(long term stability) as measured by CEX-HPLC.

| Formulation | | | | | |
|---|---|---|---|---|---|
| Composition | % Acidic Species | | | | |
| Time | Condition | Condition | Condition | Condition | Condition |
| (at 5° C.) | 1 | 2 | 3 | 4 | 5 |
| T0 | 22.9 | 25.7 | 26.5 | 26.9 | 26.7 |
| 3.5 month | 28.3 | 27.9 | 28.1 | 28.4 | 27.9 |
| 7 month | 29.2 | 28.8 | 29.1 | 29.4 | 29.4 |
| 12 month | 29.7 | 28.6 | 29.0 | 29.5 | 28.9 |
| 18 month | 28.7 | 27.3 | 27.3 | 27.6 | 27.4 |

The relative potency for bevacizumab biosimilar formulations as measured by HUVEC cell based VEGF neutralization assay were found to be within 90-110% for all conditions (condition 2-5) as compared to Condition 1 (Table 7 (ii)). This finding also confirms that the potency of the formulations (condition 2-5) is not affected due to alterations in formulation composition and is equivalent to the formulation of Avastin composition over 18 months of storage at 2-8° C.

TABLE 7 (ii)
Relative potency for bevacizumab biosimilar formulations (conditions
1 to 5) as measured by the HUVEC cell based VEGF neutralization
assay after 18 months of storage at 2-8° C.

| Sample Description | Relative Potency Compared to Condition 1 |
|---|---|
| Condition 1 | 100 |
| Condition 2 | 103 |
| Condition 3 | 101 |
| Condition 4 | 100 |
| Condition 5 | 99 |

The relative potency for bevacizumab biosimilar formulations as measured by VEGF binding Immunoassay were found to be within 90-100% for all conditions (condition 2-5) as compared to Condition 1 (Table 7 (iii)). This finding also confirms that the potency of the formulations (condition 2-5) is not affected due to alterations in formulation composition and is equivalent to the formulation of Avastin composition over 18 months of storage at 2-8° C.

TABLE 7 (iii)
Relative potency for bevacizumab biosimilar formulations
(conditions 1 to 5) as measured by the VEGF binding
Immunoassay after 18 months of storage at 2-8° C.

| Sample Description | Relative Potency Compared to Condition 1 |
|---|---|
| Condition 1 | 100% |
| Condition 2 | 95% |
| Condition 3 | 93% |
| Condition 4 | 97% |
| Condition 5 | 97% |

Samples on accelerated storage stability (30° C.) indicate that both the citrate phosphate based compositions and the acetate buffer based compositions are more stable than the bevacizumab (Avastin®) match composition (FIG. 2C and FIG. 2D; Tables 8 and 9). FIG. 2D and Table 9 indicate there are measurable covalent dimers in all five bevacizumab biosimilar compositions. All biosimilar formulation conditions (2-5) were found to have lower covalent dimers than those present in the bevacizumab match composition.

TABLE 8
Total aggregates in bevacizumab biosimilar formulations (accelerated
stability at 30° C.) as measured by SE-HPLC (neat injection).

| Formulation | | | | | |
|---|---|---|---|---|---|
| Composition | % Aggregates | | | | |
| Time | Condition | Condition | Condition | Condition | Condition |
| (at 30° C.) | 1 | 2 | 3 | 4 | 5 |
| T0 | 6.0 | 3.1 | 3.5 | 5.7 | 6.2 |
| Day 7 | 6.1 | 3.3 | 3.3 | 3.8 | 5.3 |
| Day 14 | 6.6 | 3.5 | 4.1 | 3.9 | 5 7 |
| 3.5 Months | 8.3 | 4.6 | 5.3 | 4.1 | 6.2 |

TABLE 9
Covalent dimers in bevacizumab biosimilar formulations
(accelerated stability@30° C.) as measured
by SE-HPLC (dilute injection).

| Formulation | | | | | |
|---|---|---|---|---|---|
| Condition | % Covalent Dimer | | | | |
| Time | Condition | Condition | Condition | Condition | Condition |
| (at ° 30 C.) | 1 | 2 | 3 | 4 | 5 |
| T0 | 1.7 | 1.7 | 1.7 | 1.5 | 1.4 |
| 2 Months | 3.6 | 2.8 | 3.0 | 2.3 | 2.5 |
| 3.5 Months | 4.0 | 3.2 | 3.7 | 2.7 | 3.2 |

Samples on accelerated storage stability (37° C.) indicated that both the citrate phosphate based compositions and the acetate buffer based compositions are more stable than the bevacizumab match composition (FIG. 2E and FIG. 2F; Tables 10 and 11). FIG. 2F and Table 11 indicate the presence of measurable covalent dimers in all five bevacizumab biosimilar compositions. Nevertheless, all conditions (2-5), have lower covalent dimers than those present in the bevacizumab match composition.

TABLE 10
Total aggregates in bevacizumab biosimilar formulations (Accelerated
stability at 37° C.) as measured by SE-HPLC (neat injection).

| Formulation | | | | | |
|---|---|---|---|---|---|
| Composition | % Aggregates | | | | |
| Time | Condition | Condition | Condition | Condition | Condition |
| (at 37° C.) | 1 | 2 | 3 | 4 | 5 |
| T0 | 6.0 | 3.1 | 3.5 | 5.7 | 6.2 |
| Day 7 | 6.7 | 3.4 | 4.0 | 3.9 | 5.7 |
| Day 14 | 7.3 | 3.9 | 4,6 | 4.0 | 6.2 |
| Day 21 | 7.8 | 4.2 | 4.9 | 4.3 | 6.2 |
| Day 28 | 8.3 | 4.4 | 5.1 | 4.2 | 5.6 |
| 2 Months | 9.9 | 5.2 | 5.9 | 4,6 | 6.4 |

TABLE 11
Covalent dimers in bevacizumab biosimilar formulations (accelerated
stability at 37° C.) as measured by SE-HPLC (dilute injection).

| Formulation | | | | | |
|---|---|---|---|---|---|
| Composition | % Covalent Dimer | | | | |
| Time | Condition | Condition | Condition | Condition | Condition |
| (at 37° C.) | 1 | 2 | 3 | 4 | 5 |
| T0 | 1.7 | 1.7 | 1.7 | 1.5 | 1.4 |
| 2 Months | 5.1 | 3.7 | 3.8 | 3.0 | 3.2 |

Samples on stress testing (shaking at room temperature at 150 rpm), indicate that both the citrate phosphate based compositions and the acetate buffer based compositions are more stable than the bevacizumab (Avastin®) match composition (FIG. 2G and FIG. 2H; Tables 12 and 13). The Acetate-Sucrose composition (condition 5) had a slightly higher percentage of aggregate, indicating that the pH of 5.6 is preferred for formulation stability of bevacizumab over pH 5.8. FIG. 2H and Table 13 indicate the measured covalent dimers in all five bevacizumab biosimilar compositions. All conditions (2-5), were observed to have lower covalent dimers than those present in the bevacizumab (Avastin®) match composition.

TABLE 12
Total aggregates in bevacizumab biosimilar formulations (shaking
at 150 rpm) as measured by SE-HPLC (neat injection).

| Formulation | | | | | |
|---|---|---|---|---|---|
| Composition | % Aggregates | | | | |
| Time (shaking | Condition | Condition | Condition | Condition | Condition |
| at 150 rpm) | 1 | 2 | 3 | 4 | 5 |
| T0 | 6.0 | 3.1 | 3.5 | 5.7 | 6.2 |
| Day 7 | 6.4 | 3.3 | 3.9 | 4.0 | 5.3 |
| Day 14 | 6.5 | 3.2 | 4.0 | 4.0 | 6.0 |
| Day 21 | 6.7 | 3.8 | 4.3 | 4.2 | 5.7 |

TABLE 13
Covalent dimers in bevacizumab biosimilar formulations (shaking
at 150 rpm) as measured by SE-HPLC (dilute injection).

| Formulation | | | | | |
|---|---|---|---|---|---|
| Composition | % Covalent Dimer | | | | |
| Time (shaking | Condition | Condition | Condition | Condition | Condition |
| at 150 rpm) | 1 | 2 | 3 | 4 | 5 |
| T0 | 1 7 | 1.7 | 1.7 | 1.5 | 1.4 |
| Day 21 | 2.8 | 2.2 | 2.4 | 2.0 | 2.1 |

Samples on stress testing (freeze/thaw testing) indicate that both the citrate phosphate based compositions and the acetate buffer based compositions are equivalent to the bevacizumab (Avastin®) match composition with regard to offering protection against freeze/thaw stress (FIG. 2I and FIG. 2J; Tables 14 and 15). The Acetate-Sucrose composition (condition 5) had a slightly higher percentage of aggregate, indicating that the pH of 5.6 is preferred for formulation stability of bevacizumab over pH 5.8. FIG. 2J and Table 15 indicate the presence of measurable covalent dimers in all five bevacizumab biosimilar compositions. All conditions (2-5), had lower covalent dimers than those present in the bevacizumab match composition.

TABLE 14
Total aggregates in bevacizumab biosimilar formulations (shaking
at 150 rpm) as measured by SE-HPLC (neat injection).

| Formulation | | | | | |
|---|---|---|---|---|---|
| Composition | % Aggregates | | | | |
| Freeze/thaw | Condition | Condition | Condition | Condition | Condition |
| stress testing | 1 | 2 | 3 | 4 | 5 |
| T0 | 6.0 | 3.1 | 3.5 | 5.7 | 6.2 |
| cycle 1 | 6.1 | 3.1 | 3.8 | 3.7 | 5.2 |
| RT/−20° C. | | | | | |
| cycle 3 | 6.2 | 3.2 | 3.7 | 3.6 | 5.8 |
| RT/−20° C. | | | | | |

TABLE 15
Covalent dimers in bevacizumab biosimilar formulations (Freeze/thaw
stress) as measured by SE-HPLC (dilute injection).

| Formulation | | | | | |
|---|---|---|---|---|---|
| conditions | % Covalent Dimer | | | | |
| Freeze/thaw | Condition | Condition | Condition | Condition | Condition |
| stress testing | 1 | 2 | 3 | 4 | 5 |
| T0 | 1.7 | 1.7 | 1.7 | 1.5 | 1.4 |
| cycle 1 | 1.7 | 1.7 | 1.7 | 1.6 | 1.6 |
| RT/−20° C. | | | | | |
| cycle 3 | 1.7 | 1.6 | 1.8 | 1.6 | 1.7 |
| RT/−20° C. | | | | | |

### Example 4—Biophysical Properties of Bevacizumab Biosimilar in Citrate Phosphate Buffered Trehalose and Acetate Sucrose Formulations

Biophysical properties of the four buffered biosimilar test formulations were assessed in parallel with the bevacizumab match reference formulation. Biophysical properties including but not limited to those tested by Differential Scanning Calorimetry (DSC), Dynamic light scattering (DLS), Fluorescence Spectroscopy (Int. Fl.) were assessed. Similarity of biosimilars was assessed by several orthogonal tools, with these biophysical methods being one such approach within the orthogonal analytical methods to assess biosimilarity. Orthogonal tools indicate the biophysical properties of four buffered formulations of bevacizumab biosimilar (condition 2-5) were similar or better to that of bevacizumab match composition (condition 1).

The melting temperatures and, hence, the thermal unfolding pattern for bevacizumab in all formulations is similar with a Tm1 of about 73° C. and Tm2 of about 83° C. (Table 16). This indicates that all the developed formulation conditions offer similar conformational stability to bevacizumab. Ultimately it is the long term stability study (18 months storage at 5° C.) as described in Example 3, which conclusively identifies conditions 2-5 as formulations (i.e. compositions) wherein Bevacizumab is more stable and less prone to aggregation.

TABLE 16
Conformational stability of bevacizumab biosimilar
formulations compositions as measured by DSC.

| | | Actual | | |
|---|---|---|---|---|
| Formulation | | Buffer | | |
| Condition | Sample | pH | Tm1 | Tm2 |
| 1 | Bevacizumab Match | 6.2 | 73.3 | 83.3 |
| 2 | Bevacizumab Citrate Phosphate | 5.8 | 72.8 | 82.8 |
| | pH 5.8 | | | |
| 3 | Bevacizumab Citrate Phosphate | 6.0 | 72.4 | 82.9 |
| | pH 6.0 | | | |
| 4 | Bevacizumab Acetate pH 5.6 | 5.6 | 73.5 | 83.1 |
| 5 | Bevacizumab Acetate 5.8 | 5.8 | 73.6 | 83.3 |

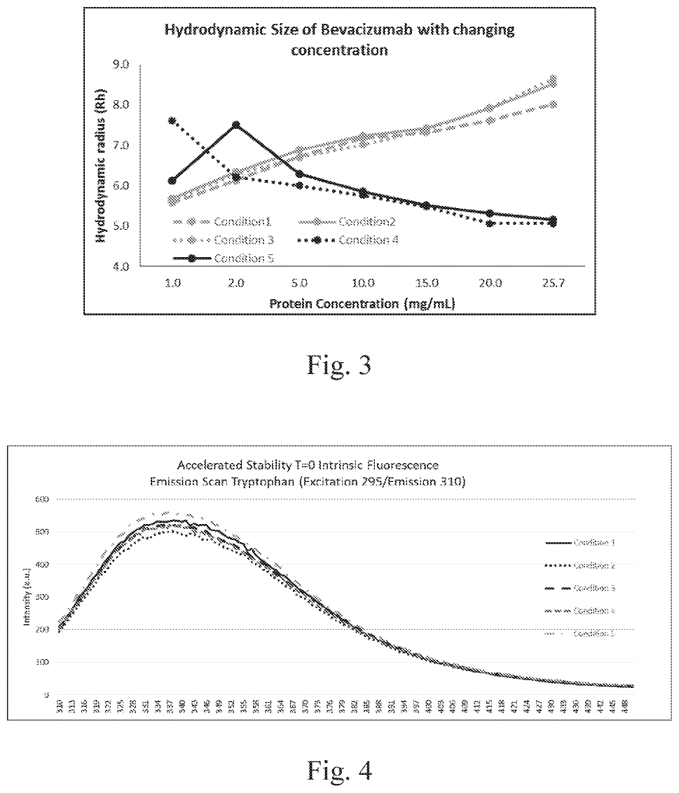

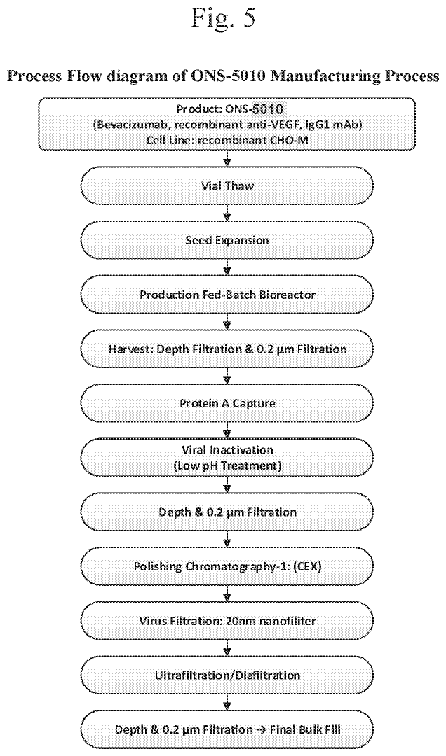

Dynamic Light Scattering (DLS) based assessment of the hydrodynamic properties of bevacizumab biosimilar in all four formulation conditions was assessed in comparison to bevacizumab match composition (condition 1). The hydrodynamic radius of bevacizumab biosimilar increases from about 6 nm to 7 nm (at a concentration of 15 mg/ml) in conditions 1-3 (Table 17, FIG. 3). The acetate conditions, 4 and 5, showed notable different size trends as compared to the bevacizumab match composition (condition 1), indicating a better colloidal stability. While the hydrodynamic size in the citrate phosphate conditions, 2 and 3, the trend was similar to the bevacizumab match condition (condition 1), the reversible aggregate and covalent dimer formation trends (long term storage over a duration of 18 months and accelerated temperature storage stability as described in example 3) indicate it offers better protection against aggregation.

TABLE 17
Hydrodynamic size (average) and diffusion coefficient (average) as measured
for different formulation conditions by DLS.

| Formulation | | | | | | | | | | |
|---|---|---|---|---|---|---|---|---|---|---|
| Target | | | | | | | | | | |
| diluted | Condition 1 | | Condition 2 | | Condition 3 | | Condition 4 | | Condition 5 | |
| Protein | Avg | Avg | Avg | Avg | Avg | Avg | Avg | Avg | Avg | Avg |
| Concentration | Rh | Diffusion | Rh | Diffusion | Rh | Diffusion | Rh | Diffusion | Rh | Diffusion |
| (mg/mL) | (nm) | Coeff (nm) | (nm) | Coeff (nm) | (nm) | Coeff (nm) | (nm) | Coeff (nm) | (nm) | Coeff (nm) |
| 1.0 | 5.6 | 4.31E−07 | 5.7 | 4.30E−07 | 5.6 | 4.24E−07 | 7.6 | 3.29E−07 | 6.1 | 2.51E−07 |
| 2.0 | 6.1 | 3.94E−07 | 6.4 | 3.80E−07 | 6.3 | 3.83E−07 | 6.2 | 3.87E−07 | 7.5 | 3.22E−07 |
| 5.0 | 6.7 | 3.59E−07 | 6.9 | 3.51E−07 | 6.7 | 3.60E−07 | 6.0 | 4.04E−07 | 6.3 | 3.82E−07 |
| 10.0 | 7.2 | 3.39E−07 | 7 2 | 3.31E−07 | 7.0 | 3.33E−07 | 5.8 | 4.20E−07 | 5.9 | 4.13E−07 |
| 15.0 | 7.3 | 3.27E−07 | 7.4 | 3.25E−07 | 7.4 | 3.21E−07 | 5.5 | 4.44E−07 | 5.5 | 4.42E−07 |
| 20.0 | 7.6 | 3.33E−07 | 7 9 | 3.06E−07 | 7 9 | 3.05E−07 | 5.1 | 4.76E−07 | 5.3 | 4.60E−07 |
| 25.7 | 8.0 | 3.02E−07 | 8.5 | 2.81E−07 | 8.7 | 2.85E−07 | 5.1 | 4.92E−07 | 5.2 | 4.73E−07 |

Note:
The Rh indicated in bold here are multimodal measurements.

Intrinsic fluorescence spectroscopy indicates all formulation conditions offering similar conformational stability to bevacizumab as the match composition (condition 1) (Table 18 and FIG. 4). The key biophysical descriptors of this test (absorbance maximum and wavelength maximum) are similar for all formulation conditions.

TABLE 18
Intrinsic fluorescence spectra for all formulation conditions
indicating the average peak maximum and the absorbance

| | | Tryptophan - 295/310 | |
|---|---|---|---|
| | | Average | |
| Formulation | | Peak max | Average |
| Condition | Formulation Description | (nm) | Absorbance |
| 1 | Bevacizumab Match | 341 | 542.6 |
| 2 | Bevacizumab Citrate Phosphate | 338 | 502.7 |
| | pH 5.8 | | |
| 3 | Bevacizumab Citrate Phosphate | 336 | 522.3 |
| | pH 6.0 | | |
| 4 | Bevacizumab Acetate pH 5.6 | 337 | 518.5 |
| 5 | Bevacizumab Acetate pH 5.8 | 336 | 557.6 |

Bevacizumab has been shown to have a significant sensitivity to even subtle changes in pH leading to increased amounts of aggregates present either in process intermediates or in the drug substance if not controlled. Antibody ONS-5010 represents a biosimilar of bevacizumab, and the ultrafiltration/diafiltration process has been improved to enhance storage stability. The specified pH range for of the final formulation for ONS-5010 is 5.9-6.3. It has been observed during ONS-5010 development that pH values approaching 6.2 and beyond carry a continued increase in % HMWS. Furthermore, bevacizumab exhibits an additional phenomenon of “reversible aggregation.” A portion of the total % HMWS species present in the drug substance over time at the prescribed storage condition (2-8° C.) will proceed towards an aggregated state until an equilibrium is reached. This presents an added concern around the shelf-life of the drug substance or product upon storage. Therefore, the development of the manufacturing process for ONS-5010 was designed primarily around understanding and maintaining an acceptable amount of % HMWS throughout. The examples below detail the development of the final unit operation in the downstream manufacturing process; ultrafiltration/diafiltration (UF/DF) followed by formulation and final filtration.

### Example 5: Conventional UF/DF

A series of experiments were performed to determine the most effective processing method. An initial set of experiments was performed to determine if a conventional UF % DF process in which ONS-5010 was concentrated and diafiltered into Final Formulation Buffer (FFB) would be feasible. The conditions are shown in Table 19.

TABLE 19
Process Variables and Analytical Results
for a Conventional TFF Process

| | Parameters | Condition (N = 3) |
|---|---|---|
| Conditions | Membrane Type | Regenerated |
| | | Cellulose, PES |
| | Membrane Cut off | 30 kD |
| | Mass Load (g/m²) | 100-150 |
| | Diafiltration | FFB, FFB + 0.02% |
| | Buffer | (v/v) PS-20 |
| | Diavolumes | ≥5 |
| | NaCl Concentration | 118-237 |
| | of Load Material | |
| | (mM) | |
| Procedure | ONS-5010 Concen- | 12-30 |
| | tration at | |
| | Diafiltration (g/L) | |
| Operating | TMP | 14-16 |
| Parameters | Feed Flux (LMH) | 240-720 |
| (Diafiltration) | Cross Flow Rate | 198-645 |
| | (LMH) | |

TABLE 20
Analytical Results for a Conventional TFF Process

| Intermediate | Run 1 | Run 2 | Run 3 |
|---|---|---|---|
| Load Material | 2.34 | 1.05 | 1.14 |
| BDS | 5.73 | 5.29 | 4.68 |
| Δ % BMWS | +3.39 | +4.24 | +3.54 |

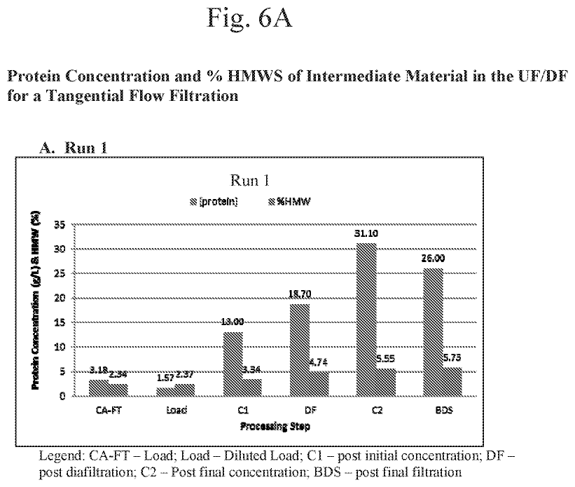

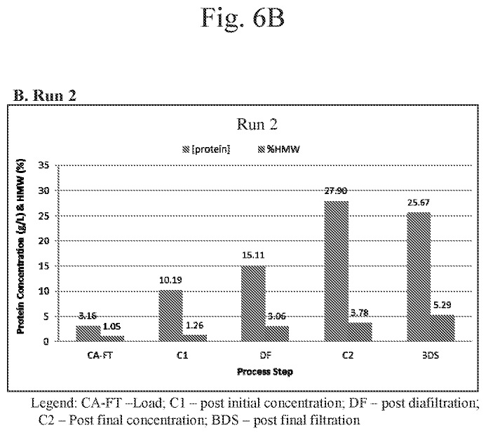

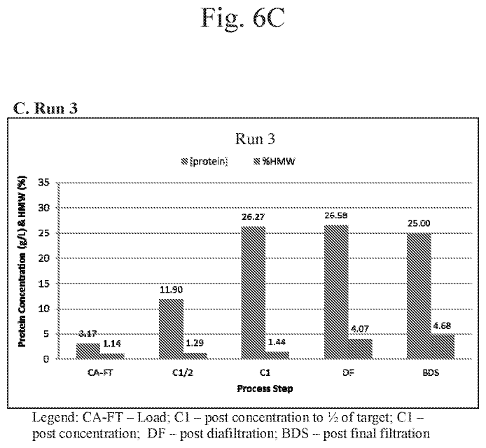

The majority of the HMWS increase was observed during the diafiltration (FIG. 6 and Table 20). In this step, material that was originally in an acetate solution with a pH of 5.0 and conductivity of 25 mS/cm was exchanged into the Final Formulation Buffer, which had a pH of 6.1 and conductivity of about 3.7 mS/cm.

### Effects of pH and Conductivity on Aggregation

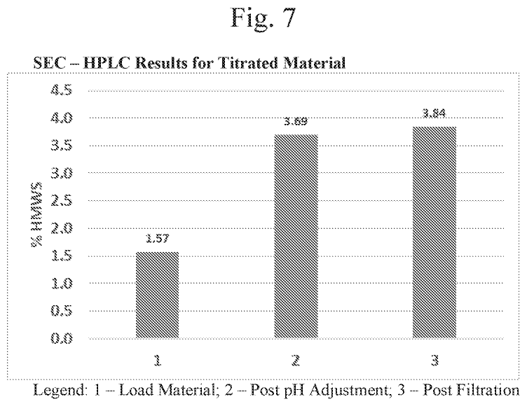

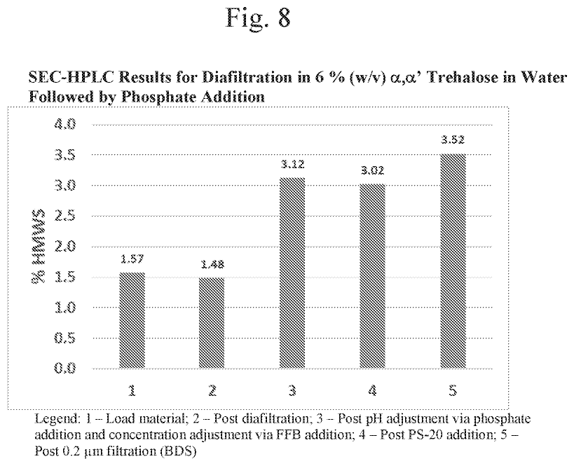

An experiment was performed to parse out the impact of pH and conductivity, using the operating conditions in Table 21. Starting material was concentrated to approximately 30 g/L and an aliquot was taken for titration to a final pH of 6.1 using 0.5 M sodium phosphate, pH 8.0 (FIG. 7). This increased the pH while having a negligible impact on the conductivity, resulting in a HMWS increase of approximately 2.2%. The remaining concentrate was diafiltered into 6% (m/v) trehalose in water, resulting in a decrease in conductivity from 25 mS/cm to approximately 1 mS/cm with no significant change in pH. Aggregate levels remained unchanged (FIG. 8; data points 1 and 2), establishing that when the conductivity changes during diafiltration and pH remains constant, the HMWS is not impacted. The recovered material from the UF/DF step in 6% (w/v) trehalose was later diluted to the final protein concentration and phosphate content using 0.5 M sodium phosphate in 6% (w/v) α,α′ trehalose and FFB without PS-20 to target an approximate protein concentration of 25 g/L and phosphate molarity of 51 mM. During these additions, the HMWS increased by 1.64% (FIG. 8), which correlates with the HMWS increase observed during the pH adjustment of the aliquot removed post concentration (FIG. 7). Polysorbate-20 was added to achieve a final concentration of 0.04% (v/v) and did not result in an increase in aggregation. Lastly, the BDS was 0.2 μm filtered using a vacuum filtration unit, which caused an additional increase in aggregation of 0.50%. This experiment demonstrated that the root cause of the majority of the HMWS increase during the UF/DF step results from the change in pH of the material.

TABLE 21
Process Variables and Analytical Results for Diafiltration
in α,α′ Trehalose

| | | Diafiltration in α,α′ |
|---|---|---|
| | Parameters | Trehalose Experiment |
| | Lot # | B140101/03-D14 |
| Conditions | Membrane Type | PES, 30 kD |
| | Mass Load (g/m²) | 150 |
| | Diafiltration | 6% (w/v) α,α′ Trehalose |
| | Buffer | in water |
| | Diavolumes | 5.3 |
| | Loading Material | Neat |
| Operating | TMP (psi) | 15 |
| Parameters | Feed Flux (LMH) | 576 |
| (Diafiltration) | Cross Flow Rate | 530 |
| | (LMH) | |

TABLE 22
SEC-HPLC Results for Diafiltration in α,α′ Trehalose

| | Diafiltration in α,α′ |
|---|---|
| Intermediate | Trehalose |
| Start Material | 1.57 |
| Prefiltered BDS | 3.02 |
| BDS | 3.14 |
| Δ % HMWS | 1.95 |

### Example 6: Two Diafiltration Steps

An experiment was designed with two diafiltration steps to determine if an immediate pH change would prove more robust than the gradual increase seen in the conventional UF/DF process. Three conditions were tested (Table 22). In the first, a phosphate spike was administered after two diavolumes of 6% (w/v) α,α′-Trehalose in water, and was followed by 5 diavolumes of FFB, without PS-20. This allowed for an immediate pH change (via the spike) but with further diafiltration that should result in a robust, reproducible pH and phosphate concentration. In the second condition, the second diafiltration step was performed in 42 mM Sodium Phosphate Monobasic, 2 mM Sodium Phosphate Dibasic, 6% (w/v) α,α-Trehalose (pH 5.5). The remaining Sodium Phosphate Dibasic was spiked in after recovery to adjust the pH to target. Finally, a condition was tested in which 2 diavolumes of 6% (w/v) α,α′-Trehalose in water were followed with 5 diavolumes of FFB. The results are shown in Table 23.

TABLE 23
Process Variables: Two Diafiltration Steps

| Parameters | Condition 1 | Condition 2 | Condition 3 |
|---|---|---|---|
| Procedure | Adjust after 2 DV | 2 DV in 6% α,α′- | 2 DV in 6% α,α′- |
| | in 6% α,α′-Trehalose, | Trehalose + 5 DV in | Trehalose, follow |
| | follow with 5 DV | sodium phosphate + | with 5 DV in FFB |
| | in FFB | Dibasic adjustment | |
| | | after recovery | |
| Number of Runs | 1 | 1 | 2 |

Conditions

| Membrane | PES | | |
|---|---|---|---|
| Surface Area (m²) | 0.02 | | 0.3 |
| Mass Load (g/m²) | 200 | 200 | 200 |
| Buffer #1 | 6% (w/v) α,α-Trehalose in water | | |
| Diavolumes | 2.7 | 2.1 | 2.3-2.4 |
| Buffer #2 | FFB without | 42 mM Sodium | FFB without |
| | PS-20 | Phosphate Monobasic, | PS-20 |
| | | 2 mM Sodium | |
| | | Phosphate Dibasic, | |
| | | 6% (w/v) α,α-Trehalose | |
| Diavolumes | 5.1 | 5.2 | 5.0-5.4 |
| Adjustment | 0.51M Sodium | Solid Sodium | N/A |
| Solution | Phosphate | Phosphate | |
| | pH 5.74 | Dibasic | |

Diafiltration Operating Parameters

| | |
|---|---|
| TMP | 15 |
| Feed Flux (LMH) | 240 |

TABLE 24
SEC-HPLC Results: Two Diafiltration Steps

| Intermediate | Condition 1 | Condition 2 | Condition 3 |
|---|---|---|---|
| Load | 0.82 | 1.17 | 1.17 |

| | | | | |
|---|---|---|---|---|
| BDS | 2.58 | 2.60 | 3.18 | 2.86 |
| Δ % HMWS | 1.76 | 1.78 | 2.01 | 1.69 |

All three conditions resulted in a significant increase in % HMWS that, although lower than in the conventional UF/DF experiment, indicated that any diafiltration performed in sodium phosphate results in an increase in % HMWS. Based on the results of this experiment, all further experiments were performed using 6% (w/v) α,α-Trehalose in water for diafiltration with the addition of phosphate and polysorbate-20 after recovery to adjust the retentate into the BDS formulation (5.8 g/L sodium phosphate monobasic, monohydrate, 1.2 g/L sodium phosphate dibasic, anhydrous, 60 g/L α,α-trehalose, 0.04% polysorbate 20, pH 6.1).

### Example 7: Impact of Final pH on the Levels of HMWS in the BDS

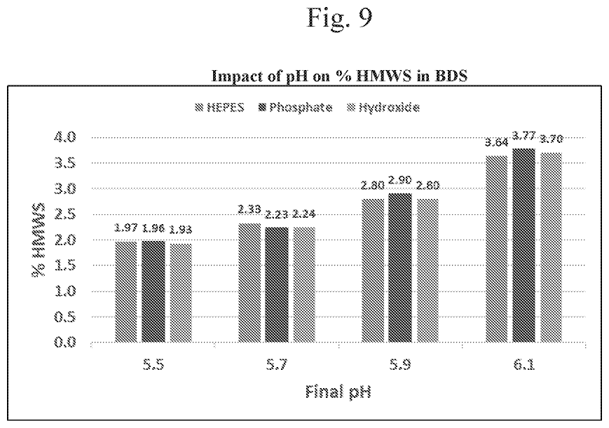

In order to confirm the impact of final pH on the levels of HMWS in the BDS, an experiment was performed in which the diafiltration was performed in 6% α,α-Trehalose in water and the recovered retentate was adjusted to pH 5.5, 5.7, 5.9, and 6.1. Sodium Phosphate (0.5 M, pH 9), 2M HEPES, pH 9, and 1N NaOH were used for adjusting the pH to see if the solution had an impact on % HMWS, or if pH was the sole driver of the increase in % HMWS (FIG. 9).

The results in FIG. 9 confirm that the pH of the BDS had a substantial impact on the % HMWS, with higher % HMWS as the pH increases. The solution components did not seem to have an impact on the % HMWS, further evidence that pH was the primary driver of the % HMWS increase.

### Example 8: Impact of Protein Concentration During PH Adjustment on % HMWS

An experiment was performed to determine if the protein concentration during pH adjustment had an impact on % HMWS. The diafiltration was performed in 6% α,α-Trehalose in water. After recovery, the retentate was diluted to 25, 28, or 30 g/L using 6% α,α-Trehalose in water. The 25 g/L and 30 g/L portions were then divided into three aliquots, and the phosphate was adjusted. The 28 g/L portion was divided into four aliquots; three underwent normal phosphate adjustment but the fourth was performed by adding powdered monobasic sodium phosphate and disodium phosphate to reach a final sodium phosphate concentration of 51 mM, pH 6.0.

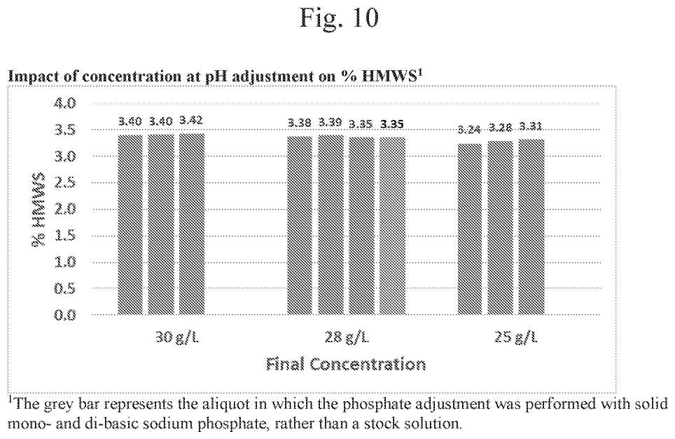

The results in FIG. 10 show that there was no impact on % HMWS when the concentration of the recovered ONS-5010 retentate varied from 30-25 g/L. The results for the 28 g/L concentration additionally indicated that the composition of the phosphate spike did not appear to impact the level of % HMWS.

### Example 9: Final UF/DF Process

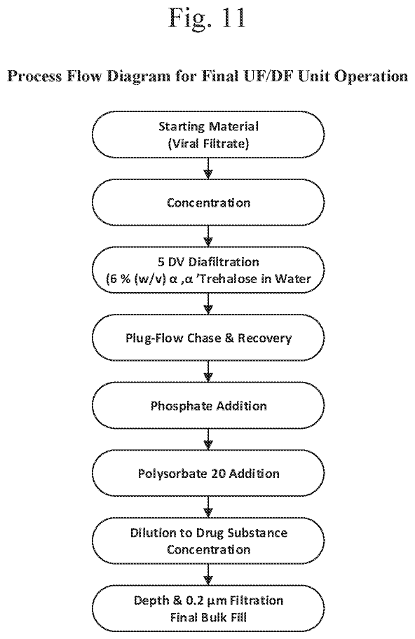

Three runs were performed to confirm the updated process at the 0.02 m² model scale using the parameters outlined in Table 27 and the process outlined in FIG. 11. The sodium phosphate and polysorbate components were added via Equations 1-4. The results from the three runs are shown in Table 26. The results from the confirmation runs were positive. The final solution contained 5.83 g/L. Sodium Phosphate monobasic and 1.22 g/L Sodium Phosphate dibasic. The final phosphate concentration in all runs was 51 mM and the final Polysorbate-20 concentration in all runs was 0.04% v/v. The % HMWS was higher in all cases than previous studies, due to a change in assays to measure the reversible as well as irreversible aggregate. The pH, conductivity, concentration, and % HMWS were all extremely consistent across the three runs indicating that the UF/DF process was robust and reproducible.

TABLE 25
Process Parameters for UF/DF and Filling of BDS

| Step Description | Process Parameter | Target | Range |
|---|---|---|---|
| Membrane | Membrane Pore Size (kDa) | 30 | |
| Preparation | Membrane Type | PES | |
| | Equilibration volume (L/m²) | 5 | NA |
| Ultrafiltration | Membrane loading (g/m²) | NA | ≤500 |
| | Feed flow rate (all operations) | 375 | ≤450 |
| | (LMH) | | |
| | Retentate Pressure (psi) | 5 | ≤25 |
| | Operating TMP (all operations) | 15 | ≤20 |
| | (psig) | | |
| | UF end concentration (g/L) | 35 | 30-40 |
| Diafiltration | Solution | 6% (w/v) α,α′-Trehalose in water | |
| | Diafiltration volumes | 5 | ≥5 |
| Concentration- | Recirc-P_{F} (psig) | NA | ≤30 |
| Depolarizaiton | Recirc-Time (min) | 10 | ≤60 |
| | Buffer flush-P_{F} (psig) | NA | ≤30 |
| | Buffer flush-Time (min) | 10 | ≤60 |
| | Protein concentration pre- | 35 | 30-40 |
| | adjustment (g/L) | | |
| | Plug-Flow Chase Volume (mL) | Calculated to achieve protein | |
| | | concentration of 30 ± 1.5 g/L | |
| Sodium Phosphate | Protein concentration (g/L), | 25 | 22.5-27.5 |
| and Polysorbate | post adjustment | | |
| 20 Addition | Final Permeate/Retentate pH | 6.2 | 5.9-6.3 |
| | Final Permeate/Retentate cond. | 3.5 | 3.5 ± 1 |
| | (mS/cm) | | |
| Filtration and | Post Concentration Depth Filter | MCE/Borosilicate Glass^{a} | |
| Final Fill | Post Concentration Depth Filter | 25 | ≤25 |
| | Capacity (L/m²) | | |
| | Terminal Filter Membrane Material | PVDF | |
| | Terminal Filter Membrane (L/m²) | 50 | ≤50 |
| | Flux (LMH) | 300 | ≤300 |

| | | |
|---|---|---|
| | Short Term Storage | ≤60 days at 5 ± 3° C. |
| Hold Time for BDS | Long Term Storage | Must be stored at −20 ± 5° C. |
| | | within 60 days from date of |
| | | manufacture |

| | | |
|---|---|---|
| Expected step yield (%) | ≥95 | ≥90 |

^{a}Filter media was Mixed Cellulose Esters with a pre-filter using borosilicate glass

$$\begin{matrix}Calculation for Sodium Phosphate Addition & \\ {V}_{0.51 M sodium Phosphate stock} = \frac{Vi}{9} \\ {V}_{i} = the volume of recovered retentate from the UF / DF & Equation 1 \\ Calculation for Polysorbate \text{-} 20 Addition & \\ {V}_{10 \% PS - 20 stock} = \frac{Vii}{249} \\ {V}_{ii} = the volume of recovered retentate after adding 0.51 M Sodium Phospate Stock Solution & Equation 2 \\ Determination of the Final Volume & \\ {V}_{final} = \frac{CiiiViii}{25} \\ {V}_{final} = the total volume required to reach the target protection concentration of 25 g \text{/} L ( L ) . \\ {C}_{iii} = the concentration of the recovered material after adding 0.51 M Sodium Phosphate and Polysorbate 20 Stock Solution ( g \text{/} L ) \\ {V}_{iii} = the volume of the recovered material after adding 0.51 M Sodium Phosphate and Polysorbate 20 stock solution and right before the dilution ( L ) . & Equation 3 \\ Calculation for Protein Concentration Diluent Addition & \\ {V}_{FFB ( no with PS - 20 )} {V}_{FFB ( no Ps - 20 )} = {V}_{final} - {V}_{iii} \\ {V}_{FFB ( with PS - 20 )} = Volume of Final Formulation Buffer with 0.04 \% ( m \text{/} v ) PS 20 required to dilute the protein concentration to 25 g \text{/} L and to achieve {V}_{final} calculated in Equation 3 ( L ) . \\ {V}_{final} = the total volume calculated in Equation 3 ( L ) . \\ {V}_{iii} = the volume of the recovered material after adding 0.51 M Sodium Phosphate and Polysorbate 20 stock solution and right before the dilution ( L ) . & Equation 4\end{matrix}$$

TABLE 26
Results from the UF/DF Confirmation Runs

| Attribute | Specification | Run 1 | Run 2 | Run 3 |
|---|---|---|---|---|
| pH | 5.9-6.3 (6.2) | 6.0 | 6.0 | 6.0 |
| Conductivity (mS/cm) | N/A | 3.98 | 3.88 | 3.84 |
| [Protein] (mg/mL) | 23-27.5 (25) | 25.2 | 24.6 | 24.9 |
| SEC-HPLC Methods | TM-0026 main peak) and TM-0033 (HMWS) | | | |
| % Main Peak (monomer) | ≥95 | 98.5 | 98.6 | 98.5 |
| % HMWS | ≤5 | 4.5 | 4.6 | 4.7 |
| Met258 | ≤2 | 1.3 | 1.2 | 1.3 |

TABLE 27
Parameters for the UF/DF Confirmation Runs

| Parameters | Values |
|---|---|
| Membrane | PES (Pall Centramate Omega) |
| Surface Area (nr) | 0.02 |
| Mass Load (g/m²) | 200 |
| Diafiltration Buffer | 6% (w/v) α,α-Trehalose in water |
| Diavolumes | 5 |
| Feed Flux (LMH) | 240 |
| TMP (psi) | 15 |

The process described in Table 27 was shown to be a robust, reproducible process, albeit with a low membrane load and feed flow rate. The membrane load is a factor of the desired process time and the permeate flux (volume of permeate over time). The permeate flux is influenced by the feed rate (LMH), retentate pressure/Trans-membrane pressure, and the viscosity of the material. In order to determine the optimum conditions necessary for the maximum amount of material in the minimum membrane area over a reasonable time frame, two optimization experiments were performed. The first was to determine the impact of flux and flow rate on product quality (as determined by increase in % HMWS), and the second was to determine an acceptable membrane load for the process.

### Example 10: Impact of Flux and Feed Rate on Product Quality

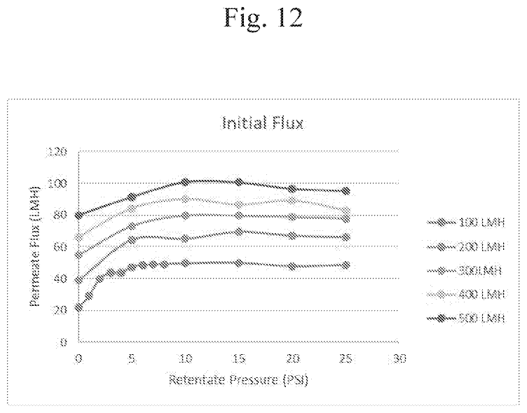

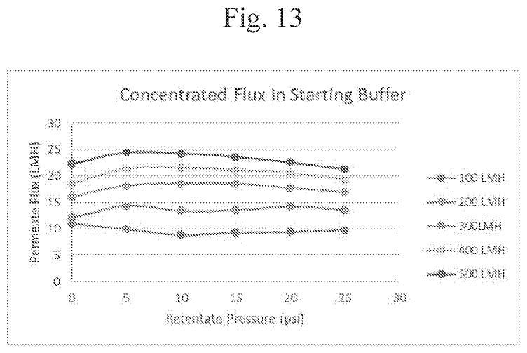

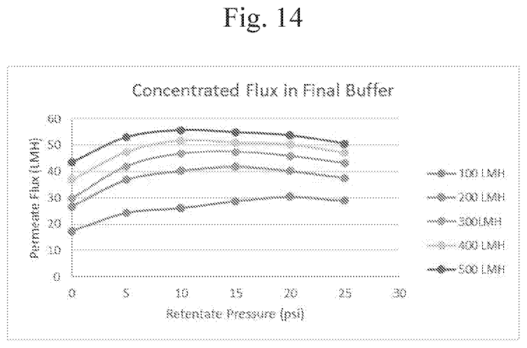

Flux rates were obtained for feed rates of 100, 200, 300, 400, and 500 LMH at retentate pressures of 0, 5, 10, 15, 20, and 25 psi. Retentate pressure control was chosen over TMP as TMP was determined by retentate pressure. The experimental parameters are shown in Table 28. Results are shown in FIGS. 12-14. FIG. 12 shows the initial permeate flux vs. retentate pressure curves for five feed flow rates at the initial ONS-5010 concentration. FIG. 13 shows the flux vs. retentate pressure curves for five feed flow rates, also at the initial ONS-5010 concentration. FIG. 14 shows the flux vs. retentate pressure curves for five feed flow rates with ONS-5010 concentrated to approximately 50 g/L. The experiment indicated that permeate flux did not increase significantly with additional retentate pressure under any of the conditions tested; therefore the retentate pressure was set at 5 psi.

TABLE 28
Impact of Flux and Feed Rate Experiment

| | |
|---|---|
| Parameters | Values |

| Membrane | PES (Pall Centramate Omega) | | |
|---|---|---|---|
| Surface Area (m²) | 0.1 | | |
| Mass Load (g/m²) | 200 | | |
| Initial Concentration | 2.3 g/L | | |
| (g/L) | | | |
| Final Concentration | 50 g/L | | |
| (g/L) | | | |
| Diafiltration Buffer | 6% (w/v) α,α-Trehalose in water | | |
| Diavolumes | 5 | | |
| Flow Rate (LMH) | 300 | 375 | 450 |

| | |
|---|---|
| Retentate Pressure (psi) | 5 |

### Example 11: Impact of Concentration on the Diafiltration Process

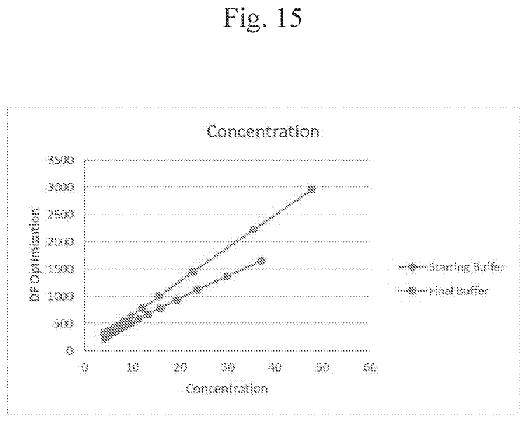

The optimum concentration for diafiltration (DF) is typically one that allows the smallest volume with the largest permeate flux. The DF Optimization parameter is determined by multiplying the concentration by the permeate flux. The maximum number obtained is the optimum concentration at which to perform the UF/DF. FIG. 15 demonstrates that in the range tested, concentration did not affect the permeate flux, i.e. the DF optimization factor continued to increase. Therefore, the previous target of diafiltration at 35 g/L was maintained. This, combined with the phosphate addition process resulted in a robust and reproducible UF/DF process.

### Example 12: Determination of Acceptable Membrane Load

A membrane load of 500 g/m² was identified as a target, and the following experiment was performed to evaluate it at multiple flow rates. These were tested against the control condition. A sample of material after the 5DV UFDF and system flush was spiked to 51 mM Phosphate and 0.04% tween 20 as per process A.0 and syringe filtered prior to SEC-HPLC analysis (Table 30). As shown in Table 30, the change in HMWS did not increase with increasing feed flow rates, and all three runs were comparable with respect to HMWS with the control run (Run 4).

TABLE 29
Determination of Acceptable Membrane Load

| Parameters | Run 1 | Run 2 | Run 3 | Control |
|---|---|---|---|---|
| Membrane | PES (Pall Centramate Omega) | | | |
| Surface Area (m²) | 0.02 | | | |
| Mass Load (g/m²) | 500 | | | 200 |
| Diafiltration Buffer | 6% (w/v) α,α-Trehalose in water | | | |
| Diavolumes | 5 | | | |
| Flow Rate (LMH) | 300 | 375 | 450 | 240 |

| | | |
|---|---|---|
| Retentate Pressure (psi) | 5 | N/A |
| TMP (psi) | N/A | 15 |

TABLE 30
Results from determination of acceptable membrane load

| Attribute | HMWS | | | HMWS | | LMWS | |
|---|---|---|---|---|---|---|---|
| Sample | Start | BDS | Δ | Start | BDS | Start | BDS |
| Run 1 | 2.2 | 6.0 | 3.8 | 97.7 | 93.9 | 0.05 | 0.05 |
| Run 2 | 2.3 | 6.1 | 3.8 | 97.7 | 93.9 | 0.05 | 0.04 |
| Run 3 | 2.7 | 6.1 | 3.4 | 97.3 | 93.9 | 0.05 | 0.04 |
| Run 4 | 2.7 | 6.4 | 3.7 | 97.2 | 93.5 | 0.06 | 0.05 |

### Example 13: Comparative Study Between ONS-5010 and Avastin Formulations

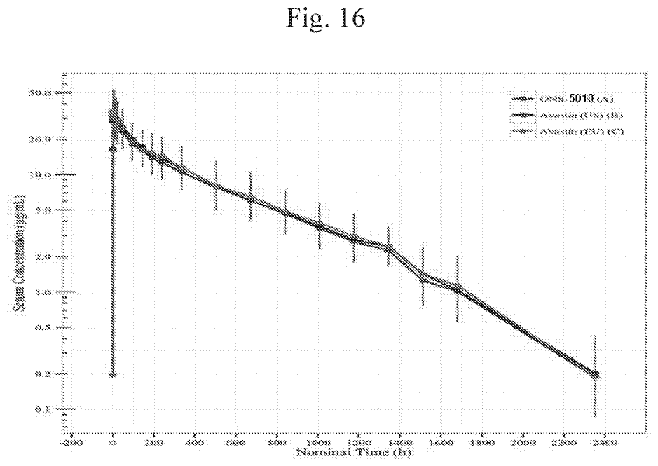

The potency of ONS-5010 was compared to alternative bevacizumab formulations including Avastin formulated for use in the U.S. and formulated for use in the E.U. FIG. 16 demonstrates the concentration-time profile of ONS-5010, U.S.-licensed Avastin, and E.U.-licensed Avastin as the mean. The vertical line at time zero denotes dosing. These results demonstrate a high degree of similarity between the three products.

1. A buffered antibody formulation comprising an antibody comprising a heavy chain comprising the amino acid sequence of SEQ ID NO: 1 and a light chain comprising the amino acid sequence of SEQ ID NO: 2 for use in treating an eye disorder in a subject.

2. The formulation for use, according to claim 1, wherein the eye disorder is a disorder of the retina, sclera, vitreous, lens, pupil, iris, cornea, choroid, optic nerve, retinal vasculature, ciliary body, or angle of the eye.

3. The formulation for use, according to claim 1, wherein the eye disorder is a disorder of the retina or choroid.

4. The formulation for use, according to claim 3, wherein the eye disorder of the retina or choroid is age-related macular degeneration, macular edema, diabetic macular edema (DME), retinopathy, diabetic retinopathy, myopic degeneration, idiopathic choroidal neovascularization, inflammatory choroidal neovascularization, retinal neovascularization, polyploidal choroidal vasculopathy, eye neovascularization, branch retinal vein occlusion (BRVO), central retinal vein occlusion, central serous chorioretinopathy, retinitis, retinitis pigmentosa, stargardt disease, usher syndrome, retinal degeneration, endophthalmitis, familial exudative vitreoretinopathy, idiopathic juxtafoveal telangiectasis, lattice degeneration, macular hole, persistent fetal vasculature, retinal artery occlusion, or retinoblastoma.

5. The formulation for use, according to claim 3, wherein the eye disorder of the retina or choroid is age-related macular degeneration, wet age-related macular degeneration, or neovascular age-related macular degeneration.

6. The formulation for use, according to claim 3, wherein the eye disorder of the retina or choroid is wet age-related macular degeneration.

7. The formulation for use, according to claim 3, wherein the eye disorder of the retina or choroid is macular edema.

8. The formulation for use, according to claim 1, wherein the buffered antibody formulation is administered to the subject orally, intravenously, intravitreally, intramuscularly, topically, subcutaneously, suprachoroidally, via eye drop, or via direct absorption through mucous membrane tissues.

9. The formulation for use, according to claim 8, wherein the buffered antibody formulation is administered to the subject by an intravitreal injection.

10. The formulation for use, according to claim 1, wherein the buffered antibody formulation is administered to the subject 1, 2, 3, 4, 5, 6, 7, 8, 9, 10, 15, 20, 28, 29, 30, or 31 times per month.

11. The formulation for use, according to claim 1, wherein the buffered antibody formulation is administered to the subject every 1, 2, 3, 4, 5, 6, 7, 8, 9, 10, 11, or 12 weeks.

12. The formulation for use, according to claim 1, wherein the buffered antibody formulation is administered to the subject for a period of time lasting 4, 8, 16, 24, 36, or 52 weeks.

13. The formulation for use, according to claim 1, wherein the buffered antibody formulation is administered to the subject for a period of time lasting 1, 2, 3, 4, 5 or 10 years.

14. The formulation for use, according to claim 1, wherein the buffered antibody formulation have a half-life of 10 to 50 days.

15. The formulation for use, according to claim 1, wherein the formulation comprises from about 10 mg/ml to about 50 mg/ml of the antibody.

16. The formulation for use, according to claim 1, wherein the formulation comprises from about 15 mg/ml to about 35 mg/ml of the antibody.

17. The formulation for use, according to claim 1, wherein the formulation comprises from about 23 mg/ml to about 27 mg/ml of the antibody.

18. The formulation for use, according to claim 1, wherein the formulation comprises from about 24 mg/ml to about 27 mg/ml of the antibody.

19. The formulation for use, according to claim 1, wherein the formulation comprises from about 25 mg/ml to about 26 mg/ml of the antibody.

20. The formulation for use, according to claim 1, wherein the formulation comprises about 25.5 mg/ml of the antibody.

21. The formulation for use, according to claim 1, wherein the formulation comprises about 25 mg/ml of the antibody.

22. The formulation for use, according to claim 1, wherein the formulation comprises from about 30 mM to about 70 mM of citrate phosphate.

23. The formulation for use, according to claim 1, wherein the formulation comprises from about 40 mM to about 60 mM of citrate phosphate.

24. The formulation for use, according to claim 1, wherein the formulation comprises from about 48 mM to about 52 mM of citrate phosphate.

25. The formulation for use, according to claim 1, wherein the formulation comprises from about 49 mM to about 51 mM of citrate phosphate.

26. The formulation for use, according to claim 1, wherein the formulation comprises from about 50 mM to about 51 mM of citrate phosphate.

27. The formulation for use, according to claim 1, wherein the formulation comprises from about 30 mM to about 70 mM of sodium phosphate.

28. The formulation for use, according to claim 1, wherein the formulation comprises from about 40 mM to about 60 mM of sodium phosphate.

29. The formulation for use, according to claim 1, wherein the formulation comprises from about 48 mM to about 52 mM of sodium phosphate.

30. The formulation for use, according to claim 1, wherein the formulation comprises from about 49 mM to about 51 mM of sodium phosphate.

31. The formulation for use, according to claim 1, wherein the formulation comprises from about 50 mM to about 51 mM of sodium phosphate.

32. The formulation for use, according to claim 1, wherein the formulation comprises sodium phosphate monobasic, sodium phosphate dibasic, or both sodium phosphate monobasic and sodium phosphate dibasic.

33. The formulation for use, according to claim 1, wherein the buffer comprises about 50 mM of sodium phosphate.

34. The formulation for use, according to claim 1, wherein the buffer comprises about 51 mM of sodium phosphate.

35. The formulation for use, according to claim 1, wherein the formulation comprises from about 120 mM to about 180 mM of trehalose.

36. The formulation for use, according to claim 1, wherein the formulation comprises from about 140 mM to about 180 mM of trehalose.

37. The formulation for use, according to claim 1, wherein the formulation comprises from about 150 mM to about 170 mM of trehalose.

38. The formulation for use, according to claim 1, wherein the formulation comprises from about 157 mM to about 161 mM of trehalose.

39. The formulation for use, according to claim 1, wherein the formulation comprises from about 158 mM to about 160 mM of trehalose.

40. The formulation for use, according to claim 1, wherein the formulation comprises about 159 mM of trehalose.

41. The formulation for use, according to claim 1, wherein the formulation comprises about 160 mM of trehalose.

42. The formulation for use, according to claim 1, wherein the formulation comprises from about 0.02% (v/v) to about 0.06% (v/v) of polysorbate 20.

43. The formulation for use, according to claim 1, wherein the formulation comprises from about 0.03% (v/v) to about 0.05% (v/v) of polysorbate 20.

44. The formulation for use, according to claim 1, wherein the formulation comprises about 0.04% (v/v) of polysorbate 20.

45. The formulation for use, according to claim 1, wherein the formulation has a pH of about 5.6.

46. The formulation for use, according to claim 1, wherein the formulation has a pH of about 5.8.

47. The formulation for use, according to claim 1, wherein the formulation has a pH of about 6.

48. The formulation for use, according to claim 1, wherein the formulation has a pH of about 6.1.

49. The formulation for use, according to claim 1, wherein the buffer comprises from about 11 mM to about 19 mM of sodium acetate.

50. The formulation for use, according to claim 1, wherein the buffer comprises from about 13 mM to about 17 mM of sodium acetate.

51. The formulation for use, according to claim 1, wherein the buffer comprises from about 13 mM to about 16 mM of sodium acetate.

52. The formulation for use, according to claim 1, wherein the buffer comprises about 15 mM of sodium acetate.

53. The formulation for use, according to claim 1, wherein the formulation comprises from about 165 mM to about 185 mM of sucrose.

54. The formulation for use, according to claim 1, wherein the formulation comprises from about 170 mM to about 180 mM of sucrose.

55. The formulation for use, according to claim 1, wherein the formulation comprises from about 174 mM to about 176 mM of sucrose.

56. The formulation for use, according to claim 1, wherein the formulation comprises about 175 mM of sucrose.

57. A kit comprising:
a) a buffered antibody formulation comprising an antibody comprising a heavy chain comprising the amino acid sequence of SEQ ID NO: 1 and a light chain comprising the amino acid sequence of SEQ ID NO: 2; and
b) instructions for administering the antibody formulation in a method for treating an eye disorder.

58.-64. (canceled)


SEQUENCE LISTING (4 sequences)

SEQ ID NO: 1 - protein, 453 aa, artificial sequence
  Bevacizumab Heavy Chain IgG1
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Bevacizumab Light Chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 123 aa, Artificial Sequence [sequence omitted]

SEQ ID NO: 4 - protein, 108 aa, Artificial Sequence
  Bevacizumab Light Chain Variable Region
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR